(19) United States
(12) Patent Application Publication
(10) Pub. No.: US 2024/0287516 A1
(43) Pub. Date: Aug. 29, 2024

(54) COMPOSITIONS AND METHODS OF USE FOR MUTATED HOTAIR IN THE TREATMENT OF CANCERS

(71) Applicant: The Regents of the University of Colorado, a body corporate, Denver, CO (US)

(72) Inventors: Aaron JOHNSON, Denver, CO (US); Allison SWAIN, Aurora, CO (US); Justin ROBERTS, Denver, CO (US)

(21) Appl. No.: 18/506,637

(22) Filed: Nov. 10, 2023

Related U.S. Application Data
(63) Continuation of application No. PCT/US22/29068, filed on May 12, 2022.
(60) Provisional application No. 63/187,835, filed on May 12, 2021.

(51) Int. Cl. C12N 15/113; A61K 45/06

(52) U.S. Cl. CPC C12N 15/113; A61K 45/06; C12N 2310/113; C12N 2320/31

(57) ABSTRACT

Embodiments of the instant disclosure relate to novel compositions and methods for treating cancer in a subject. In certain embodiments, long noncoding RNAs (lncRNAs) are modified to reduce or eliminate methylation at target nucleotides within the lncRNAs to modulate activities such as tumor promoting activities. In some embodiments, the lncRNAs includes HOTAIR (HOX (homeobox) transcript antisense intergenic RNA) or fragment thereof, where one or more nucleotides are substituted to reduce or eliminate methylation at the one or more nucleotide positions. In certain embodiments, the HOTAIR methylation site includes, but is not limited to, a modification at adenine 783 (A783) of SEQ ID. NO:1 or fragment thereof or equivalent position thereof. In other embodiments, modified lncRNAs disclosed herein can be used to treat a health condition where naturally-occurring lncRNAs have adverse effects. In some embodiments, a composition including modified HOTAIR can be used to treat a subject with cancer.

### PRIORITY

This U.S. Continuation Application claims priority to International Application PCT/US2022/029068, filed May 12, 2022, which claims priority to U.S. Provisional Patent Application No. 63/187,835, filed May 12, 2021. These applications are incorporated herein by reference in their entireties for all purposes.

### FUNDING

This invention was made with government support under grant number W81XWH-18-1-0023 awarded from the Department of Defense and grant number R35GM119575 awarded from the National Institutes of Health (NIH). The government has certain rights in the invention.

### FIELD

Embodiments of the instant disclosure relate to novel constructs, compositions and methods for treating cancer in a subject. In certain embodiments, long noncoding RNAs (lncRNAs) are modified to reduce or eliminate methylation at target nucleotides within the lncRNAs in order to affect methylation and modulate activity. In some embodiments, the lncRNAs includes HOTAIR (HOX (homeobox) transcript antisense intergenic RNA) or a fragment thereof.

### STATEMENT REGARDING SEQUENCE LISTING

The instant application contains a Sequence Listing which has been submitted via PatentCenter, and created on Jan. 22, 2024, referred to as 2024-1-25_106549-775964_CU5612H-US1_SL.xml that is 94,000 bytes in size having 76-sequences and is incorporated herein by reference in its entirety for all purposes.

### BACKGROUND

Increasing evidence has indicated that long noncoding RNA (lncRNA) is a new class of molecules associated with the development and progression of cancer and other health conditions. Knowledge of the mechanisms of lncRNAs remains unclear. Several well-studied lncRNAs have provided important clues about the biology and human health relevance of these molecules. A few key functional and mechanistic themes have begun to be uncovered but additional research is required to understand the importance of these molecules and how to regulate them. It is known that lncRNAs interact with proteins, such as epigenetic modifiers, transcriptional factors/coactivators, and RNP complexes as well as other proteins, to regulate the related biological processes. It is also known that certain lncRNAs play a role in tumor progression. There is an unmet need for identifying methods for inhibiting function of lncRNAs in order to reduce or eliminate their contributions to certain cancers.

### SUMMARY

Embodiments of the instant disclosure relate to novel constructs, compositions and methods for treating cancer in a subject. In other embodiments, compositions, constructs, and methods are disclosed to reduce or prevent metastasis of a tumor in a subject in need thereof. In certain embodiments, long noncoding RNAs (lncRNAs) can be modified to reduce or eliminate methylation at target nucleotides within the lncRNAs in order to modulate activity. In other embodiments, compositions and methods disclosed herein can be used to block methylation of one or more site in lncRNAs to modulate activity of the lncRNAs. In some embodiments, the lncRNAs disclosed herein include HOTAIR (HOX (homeobox) transcript antisense intergenic RNA) or a biologically active fragment thereof or a biologically active fragment thereof having at least one methylation site. In accordance with these embodiments, the lncRNAs (e.g., HOTAIR) can be altered where one or more methylation site(s) can be mutated or substituted or blocked from methylation to reduce or eliminate methylation at the one or more methylation site(s) in the lncRNAs (e.g., HOTAIR).

In some embodiments, compositions and constructs disclosed herein modify N6-methyladenosine (m6A) sites in HOTAIR where adenine can be removed or substituted by a different nucleotide incapable of being methylated. In accordance with these embodiments, adenine at one or more m6A site in HOTAIR can be replaced by uracil, guanine, or cytosine to eliminate methylation at the site. In certain embodiments, substitution can be an adenine to uracil substitution. In some embodiments, a targeted HOTAIR methylation site includes, but is not limited to, a substitution, mutation, or modification at adenine 783 (A783) of the HOTAIR sequence represented by SEQ ID. NO: 1 or fragment thereof, or equivalent position thereof. In certain embodiments, an A783U substitution in HOTAIR is referred to as an ‘antimorph’ sequence in part because certain activities of an antimorph HOTAIR are opposite of the WT HOTAIR. In other embodiments, modified, mutated, synthetically generated, or substituted lncRNAs disclosed herein form part of a composition for use in treating or reducing or eliminating side effects of a health condition where their respective naturally-occurring lncRNAs have adverse effects. In some embodiments, a composition including modified HOTAIR as disclosed herein can be used to treat a subject with cancer.

In other embodiments, other methylation sites can be modified blocked or edited in HOTAIR in order to modulate HOTAIR activity including, one or more adenine nucleotide substitutions or edits at one or more of nucleotide position 48, 102, 143, 215, 620, 655, 722, 936, 1394, 1579, 1663, 1722, or 1739 of SEQ ID. NO: 1 or equivalent position thereof of HOTAIR or fragment thereof, and eliminating methylation at the one or more of nucleotide position 48, 102, 143, 215, 620, 655, 722, 936, 1394, 1579, 1663, 1722, or 1739 or equivalent position thereof. In accordance with these embodiments, a synthetic polynucleotide can contain one or more m6A nucleotide substitutions at one or more adenine positions including, but not limited to, 783, and one or more of, 48, 102, 655, 722, or 1739 of SEQ ID. NO: 1 or equivalent position thereof. In some embodiments, a mutated or modified HOTAIR lncRNAs can be designed where one or more m6A methylation sites can be mutated in a particular subject as a personalized treatment approach to a subject having cancer. a particular type of cancer (e.g. breast cancer) or a particular type of metastatic cancer at risk of metastasizing, having aberrant HOTAIR expression and/or activity.

In certain embodiments, a synthetic polynucleotide or mutated HOTAIR polynucleotide or fragment thereof can further include a cell targeting agent or moiety. In some embodiments, a cell targeting agent or moiety can include a cancer cell targeting agent or moiety. In other embodiments, a synthetic polynucleotide or mutated HOTAIR polynucleotide or fragment thereof can further include a cell penetrating moiety (e.g., tat or similar or mutant equivalent thereof)

In other embodiments, one or more antisense oligonucleotide (ASO) capable of pairing by complementary interaction with HOTAIR (HOX (homeobox) transcript antisense intergenic RNA) is contemplated. In accordance with these embodiments, one or more ASO capable of pairing to one or more methylation sites in HOTAIR is disclosed herein in order to reduce or eliminate methylation at the one or more methylation sites. In one embodiment, one or more ASO that recognizes and pairs with a methylation site in HOTAIR can include, but is not limited to, recognition and pairing by complementarity to adenine position 783 of SEQ ID. NO: 1, fragment thereof or equivalent position thereof and where the one or more ASO blocks methylation of position A783 or equivalent position thereof. In other embodiments, one or more ASO that recognizes and pairs with a methylation site in HOTAIR can include, but is not limited to, recognition and pairing by complementarity to all or part of positions beginning at position 723 up to position 808 of SEQ ID. NO: 1, fragment thereof or equivalent position thereof wherein methylation of adenine position 783 of SEQ ID. NO: 1, is reduced or blocked by one or more ASO. In certain embodiments, one or more ASO can be designed that is particular to a patient having cancer with aberrant HOTAIR expression or activity. In accordance with these embodiments, personalized ASOs pair with HOTAIR including pairing with one or more m6A HOTAIR sites to block methylation and reduce tumor promoting activities and/or induce tumor suppressor activities of HOTAIR in the patient. In certain embodiment, the one or more ASO is at least ten (10) nucleotides in length. In other embodiments, the one or more ASO can be fifty (50) nucleotides in length or less. In some embodiments, one or more ASOs can include a uracil that binds to the adenosine at position 783 of SEQ ID. NO: 1 or equivalent position thereof. In other embodiments, the one or more ASO further includes a cell targeting agent or moiety. In other embodiments, the ASO can further include at least one chemical modification to the ASO phosphodiester backbone. In yet other embodiments, the at least one chemical modification to the ASO phosphodiester backbone includes, but is not limited to, phosphorothioate DNA, phosphorodiamidate morpholino (PMO), peptide nucleic acid, tricyclo-DNA, ribose substitution 2′-O-methyl (2′-OMe), ribose substitution 2′-O-methoxyethyl (2′-MOE), ribose substitution locked nucleic acid, or any combination thereof.

In other embodiments, a CRISPR genome editing approach can be used to edit one or more m6A sites in HOTAIR, including but not limited to. editing A783 in HOTAIR in order to alter functional HOTAIR interactions downstream such as chromatin association and gene repression, for example. In certain embodiments, YTHDC1 interactions with HOTAIR can be disrupted. In accordance with these embodiments, a CRISPR genome editing system known in the art can be designed to recognize certain regions of the HOTAIR gene and for example, can be used to edit A783 represented by SEQ ID NO. 1 or equivalent region thereof to edit adenine to a uracil. In some embodiments, a guide RNA can be directed to bind upstream from the site of interest in order to edit HOTAIR. In certain embodiments, a CRISPR/Cas system can be delivered using a viral vector such as an attenuated viral vector known in the art.

In other embodiments, compositions disclosed herein include one or more mutated HOTAIR construct or biologically active fragment thereof and/or one or more ASO derived from and capable of binding HOTAIR (e.g. that associates with or blocks interactions with A783). Some embodiments disclosed herein include pharmaceutical compositions and further include a pharmaceutically acceptable excipient or agent in combination with one or more mutated HOTAIR construct or biologically active fragment thereof and/or one or more ASO derived from and capable of binding HOTAIR (e.g. that associates with or blocks interactions with A783). In certain embodiments, compositions disclosed herein can include buffers and/or reagents for stabilizing one or more synthetic polynucleotide (e.g., HOTAIR mutants) having one or more substitutions or an anti-sense oligonucleotide thereof. In some embodiments, the mutated HOTAIR or one or more ASO generated to associate with HOTAIR and interfere with methylation can be stored in a standard buffer or other suitable buffer. In other embodiments, the buffer pH can be about 6.5 to about 8.5 or about 7.0 to about 8.0. In yet other embodiments, other agents can be provided to supplement the buffer and enhance stabilization of these molecules such as salt, poloxamers or the like, EDTA or the like.

In certain embodiments, methods for treating or reducing side effects a health condition having aberrant expression of HOTAIR are contemplated. In some embodiment, the health condition includes cancer. In certain embodiments, methods include, but are not limited to, administering a composition including one or more mutant HOTAIR or ASO derived therefrom disclosed herein to the subject and treating the cancer or reducing or eliminating metastasis in the subject. In other embodiments, the cancer includes a solid tumor that is currently or is suspected of expressing or overexpressing HOTAIR. In certain embodiments, the cancer includes, but is not limited to, breast, prostate, endometrial, pancreatic, glioma, lung (e.g. small cell lung), liver, stomach, colon, intestinal, other gastric cancer, ovarian or any combination thereof or any metastasizing tumor thereof. In certain embodiments, the targeted cancer includes breast cancer expressing aberrant levels of HOTAIR.

In certain embodiments, the composition includes constructs having at least an A783U mutation in HOTAIR represented by SEQ. ID. NO.1, or equivalent position of HOTAIR thereof or fragment thereof. Where, in the composition induces one or more of: reduces or eliminates HOTAIR cancer-promoting activity or activities, reduces HOTAIR downstream interactions, induces cancer suppression, reduces cancer cell expansion, reduces or eliminates metastasis in the subject and treats the cancer in the subject. In certain embodiments, the cancer being treated includes breast cancer.

In other embodiments, a subject being treated for cancer using a composition containing mutant HOTAIR and/or one or more ASOs can further be treated with one or more standard cancer treatments. In accordance with these embodiments, the one or more standard treatments can include, but are not limited to, radiation, surgery, chemotherapy, immunotherapy, hormone therapy, stem cell therapy, bone marrow transplantation, or other method for treating cancer.

In some embodiments, administering compositions disclosed herein can include administering a composition intravenously, by continuous infusion over a predetermined period of time, subcutaneously, intraocularly, topically, intradermally, intranasally, directly into the tumor or other mode. In yet other embodiments, compositions can be administered to a subject daily, every other day, bi-weekly, weekly, bi-monthly, monthly, every other month or other time period determined by a healthcare provider.

In other embodiments, YTHDC1 interactions with HOTAIR can be blocked or modified in a subject having cancer by at least one of blocking or reducing methylation of HOTAIR and/or blocking YTHDC1 by an agent capable of reducing or modifying HOTAIR/YTHDC1 interaction. In certain embodiments, YTHDC1 can be blocked from interacting with methylation site A783 of HOTAIR represented by the sequence of SEQ ID NO.1.

Other embodiments disclosed herein concern kits for transporting or storage of at least one synthetic HOTAIR polynucleotides having one or more substitution or mutation to reduce or prevent adenosine methylation or one or more anti-sense oligonucleotide (ASO) that pairs with or blocks one or more HOTAIR methylation sites; and at least one container. Kits can further include devices, buffers and other components. In certain embodiments, kits are provided for use in treating or reducing the size of, or reducing expansion of a tumor.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1H illustrate m6A modification of LncRNA HOTAIR in accordance with certain embodiments of the present disclosure. FIG. 1A represents a schematic diagram illustrating a general model for HOTAIR mechanisms. FIGS. 1B and 1C represent exemplary plots demonstrating representative results from immunoprecipitating m6A RNA with an m6A antibody or a control antibody in two different cell lines overexpressing HOTAIR. FIG. 1D represents a schematic diagram illustrating a HOTAIR transcript with exemplary m6A sites. FIG. 1E represents a representative plot indicating the number of HOTAIR transcripts in an exemplary cancer cell line expressing WT HOTAIR, mutant HOTAIR, or control RNA. FIG. 1F represents a representative plot demonstrating some results from immunoprecipitating m6A RNA with an m6A antibody or a control antibody in two different cell lines overexpressing WT HOTAIR or HOTAIR A783U. FIG. 1G represents a representative plot indicating the doubling time of an exemplary cancer cell line expressing WT HOTAIR, mutant HOTAIR, or control RNA. FIG. 1H represents a representative plot of cell invasion in a cancer cell line expressing WT HOTAIR, mutant HOTAIR or control RNA, according to various aspects of the disclosure.

FIGS. 2A-2G illustrate YTHDC1 interaction with HOTAIR in accordance with certain embodiments of the present disclosure. FIGS. 2A and 2B include a representative immunoprecipitation of the effector protein YTHDC1 using an YTHDC1 antibody or control in MCF-7 (A) or MDA-MB-231 cells. (B) with wild-type HOTAIR. FIG. 2C includes a representative immunoprecipitation of the effector protein YTHDC1 with wild-type and A783U mutant HOTAIR. FIG. 2D is a representative immunoprecipitation of the effector protein YTHDC1 in a breast cancer cell line with only A783 methylated using qRT-PCR analyses at targeted regions of the HOTAIR RNA. FIGS. 2E-2G represent schematic of in vitro methylated RNA pulldown (2E), an exemplary western blot result from a pulldown of the in vitro transcribed HOTAIR sequence around A783 using a biochemical enzymatic methylation and probing for pulldown of effector protein YTHDC1 (2F), and quantification (2G), according to various aspects of the disclosure, according to various aspects of the disclosure.

FIGS. 3A-3G illustrate YTHDC1 regulation of HOTAIR activity and stability in accordance with certain embodiments of the present disclosure. FIGS. 3A and 3B represent a representative Western blot and quantification of protein expression of YTHDC1 in various cell lines where the protein is up- or down-regulated. FIG. 3C represents an illustrative plot of doubling time in various cell lines where YTHDC1 is up- or down-regulated. FIGS. 3D and 3E represent representative plots of chromatin association of WT or A783U HOTAIR in various cell lines where YTHDC1 is unmanipulated (3D) or up- or down-regulated (3E). FIG. 3F represents a representative plot of HOTAIR levels in specific transgene constructs with additional m6A sites mutated. FIG. 3G represents a representative plot of HOTAIR levels in various cell lines where YTHDC1 is up- or down-regulated.

FIGS. 4A-4I illustrate YTHDC1 mediation of transcriptional repression by HOTAIR in accordance with certain embodiments of the present disclosure. FIG. 4A represents a schematic diagram of the HOTAIR tethering reporter gene system. FIGS. 4B and 4C represent representative plots of reporter luciferase RNA and protein levels with or without HOTAIR tethered to the reporter. FIGS. 4D and 4E represent representative western blots of METTL3 or YTHDC1 in cells treated with control or siRNAs targeting those genes. FIG. 4F-4I illustrate exemplary plots of reporter luciferase RNA (4F and 4H) and protein levels (4G and 4I) with siRNA treatment to knock down METLL3 (4F and 4G) or YTHDC1 (H and I).

FIGS. 5A-5E illustrate HOTAIR-mediated gene expression changes in breast cancer that were altered by mutation of A783 in accordance with certain embodiments of the present disclosure. FIG. 5A represents an exemplary heatmap of the expression of differentially expressed genes (DEGs) between breast cancer cells overexpressing WT HOTAIR compared to a control RNA. FIG. 5B represents representative plots of qRT-PCR analysis of genes downregulated with HOTAIR overexpression. FIG. 5C represents representative plots of qRT-PCR analysis of genes upregulated with HOTAIR overexpression. FIG. 5D represents an exemplary heatmap of the expression of differentially expressed genes (DEGs) between breast cancer cells overexpressing a mutated HOTAIR compared to a control RNA. FIG. 5E represents an exemplary Venn diagram of the number of Differentially Expressed Genes (DEGs) between cancer cells overexpressing WT HOTAIR, a mutant HOTAIR, or a control RNA.

FIGS. 6A-6G illustrate tethering YTHDC1 to A783U mutant HOTAIR in accordance with certain embodiments of the present disclosure. FIG. 6A represents a schematic diagram of an engineered RNA-targeting CRISPR protein, dCasRx, fused to the effector protein YTHDC1 and guided to the region of HOTAIR A783. FIG. 6B represents a representative Western blot to probe for expression of dCasRx-YTHDC1 in cells expressing wild-type or mutant HOTAIR, dCasRx-YTHDC1, and a HOTAIR or non-targeting (NT) guide RNA. FIG. 6C represents a representative plot of a qRT-PCR to test levels of HOTAIR RNA in cells expressing wild-type or mutant HOTAIR, dCasRx-YTHDC1, and a HOTAIR or non-targeting (NT) guide RNA. FIG. 6D represents a representative plot of a qRT-PCR to test levels of chromatin-associated HOTAIR RNA in cells expressing wild-type or mutant HOTAIR, dCasRx-YTHDC1, and a HOTAIR or non-targeting (NT) guide RNA. FIGS. 6E and 6F represent doubling time (E) and Matrigel invasion (F) in cells expressing wild-type or mutant HOTAIR, dCasRx-YTHDC1, and a HOTAIR or non-targeting (NT) guide RNA. FIG. 6G represents the change in selected HOTAIR target gene expression level change with HOTAIR relative to NT gRNA, in cells expressing wild-type or mutant HOTAIR and dCasRx-YTHDC1.

FIGS. 7A-7B illustrate schematic diagrams depicting the HOTAIR mechanism in accordance with certain embodiments of the present disclosure. FIG. 7A represents a schematic diagram illustrating interpretation of experiments from FIGS. 6A-6G. FIG. 7B represents a schematic diagram illustrating specific findings that elaborate on the HOTAIR mechanism.

FIGS. 8A-8C illustrate schematic diagrams depicting a representative investigation of m6A in HOTAIR in accordance with certain embodiments of the present disclosure. FIG. 8A represents CVm6A visualization of HOTAIR m6A RIP experiments. FIG. 8B represents a schematic illustrating the meCLIP protocol. FIG. 8C represents an illustration of an RNA structure surrounding HOTAIR A783.

FIGS. 9A-9C illustrate that HOTAIR was m6A modified by METTL3/14 and regulated proliferation of TNBC cells in accordance with certain embodiments of the present disclosure. FIG. 9A represents levels of HOTAIR is various cell lines made from patient-derived xenografts of breast tumors. FIG. 9B represents an exemplary western blot of knockdown of m6A methyltransferase components METTL3, METTL14, and WTAP and subsequent immunoprecipitating m6A RNA with an m6A antibody. FIG. 9C represents raw Incucyte data and doubling times of clones from cells expressing WT HOTAIR, mutant, or control.

FIGS. 10A-10E illustrate that hnRNP B1 and YTHDF1/2 did not directly interact with HOTAIR m6A in accordance with certain embodiments of the present disclosure. FIGS. 10A and 10B represent analysis of overlap between hnRNP B1 and m6A. FIG. 10C represents the HOTAIR RNA sequence relative to RT-qPCR primers and previous hnRNP B1 CLIP data.

FIGS. 10D and 10E represents an exemplary immunoprecipitation of the effector proteins YTHDF1 and YTHDF2 in a breast cancer cell line with only A783 methylated using qRT-PCR analyses at targeted regions of the HOTAIR RNA.

FIGS. 11A-11D illustrate YTHDC1 expression levels in breast cancer regulate predictive nature of HOTAIR expression in accordance with certain embodiments of the present disclosure. FIGS. 11A and 11B represent Kaplan-Meier plots of high or low HOTAIR expressing breast cancer patient samples with high (A) or low (B) YTHDC1 mRNA, separated by overall survival. FIGS. 11C and 11D represent YTHDC1 mRNA (C) and protein (D) levels in publicly-available breast cancer patient sample data.

FIGS. 12A-12D illustrate that fractionation of MDA-MB-231 cells were a model for effects of YTHDC1 dosage and proliferation effects of multiple m6A mutants in accordance with certain embodiments of the present disclosure. FIGS. 12A and 12B represent cellular fractionation results via western blot of indicated proteins in cell lines with WT or mutant HOTAIR or control (12A) and also manipulated levels of YTHDC1 (12B). FIG. 12C represents a schematic illustration of a model based off results from FIGS. 3A-3G. FIG. 12D represents doubling time of HOTAIR-expressing cells with additional m6A site mutants, as in FIG. 3F.

FIGS. 13A-13C illustrate that HOTAIR was stable upon YTHDC1 knockdown in 293T HOTAIR-tethered reporter cell lines in accordance with certain embodiments of the present disclosure. FIGS. 13A and 13B represent HOTAIR and MS2 tag levels in cells treated with siRNA to METTL3 or YTHDC1. FIG. 13C represents a schematic illustration of a model based off of results from FIGS. 4A-4I.

FIGS. 14A-14F illustrate that expression of A783U HOTAIR induced opposite gene expression changes compared to WT HOTAIR in accordance with certain embodiments of the present disclosure. FIG. 14A represents a heatmap of all differentially-expressed genes between WT HOTAIR and A783U mutant. FIGS. 14B and 14C represent Venn diagrams depicting overlap of genes in A783U changing in the same (B) or opposite (C) direction as in WT HOTAIR. FIG. 14D represents results from experiments in FIGS. 6A-6G for genes that are upregulated by WT HOTAIR. FIG. 14E represents doubling time in cells expressing wild-type and an additional copy of mutant HOTAIR or control, testing dominant negative activity. FIG. 14F represents select HOTAIR target gene expression analysis in cells expressing wild-type and an additional copy of mutant HOTAIR or control, testing dominant negative activity.

### DEFINITIONS

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals.

As used herein, “treat,” “treating” or “treatment” can refer to treating, reversing, ameliorating, or inhibiting onset or progression of a health condition or disease or a symptom of the health condition or disease.

As used herein, “polynucleotide,” “nucleic acid” or “nucleic acid molecule” or “nucleic acid sequence” include deoxyribonucleic acid (DNA) or ribonucleic acid (RNA), mRNA, oligonucleotides, and the like.

As used herein, “vector”, “expression vector” or “construct” refers to a nucleic acid used to introduce polynucleotides into a cell that has regulatory elements to provide expression of the heterologous nucleic acids in the cell. Vectors include but are not limited to plasmid, minicircles, yeast, and/or viral genomes. In some alternatives, the vectors are plasmid, minicircles, or viral genomes. In some alternatives, the vector is a viral vector. In some embodiments, the viral vector is a lentivirus. In some alternatives, the vector is a lentiviral vector. In some embodiments, the vector is a foamy viral vector, adenoviral vectors, retroviral vectors or lentiviral vectors.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the instant disclosure relate to novel constructs, compositions and methods for treating cancer in a subject. In some embodiments, novel constructs, compositions, and methods for reducing cancer cell expansion and/or metastasis. In certain embodiments, long noncoding RNAs (lncRNAs) are modified to reduce or eliminate methylation at target nucleotides within the lncRNAs to modulate activity of the lncRNAs. In some embodiments, the lncRNAs disclosed herein include HOTAIR (HOX (homeobox) transcript antisense intergenic RNA) or a biologically active fragment thereof, where one or more methylation sites are mutated, modified or substituted to reduce or eliminate methylation at the one or more methylation sites in HOTAIR.

N6-methyladenosine (m6A) modification of RNA are important in normal and cancer biology, but knowledge of N6-methyladenosine functions in long noncoding RNAs (lncRNAs) is limited. In certain embodiments disclosed herein, it is demonstrated that interference of adenosine methylation by adenosine substitution, mutation, or modification in HOTAIR impairs cancer cell proliferation and invasion such as metastasis. In some embodiments, m6A prevention affected the ability of HOTAIR to localize to chromatin and orchestrate cancer-associated gene expression changes. HOTAIR normally interacts with nuclear m6A reader YTHDC1 and YTHDC1-HOTAIR is required for chromatin localization and gene repression. In some embodiments, constructs disclosed herein demonstrate methylation of HOTAIR plays a crucial role in cancer progression and metastasis and preventing methylation of one or more targeted sites in HOTAIR disclosed herein regulates its function in cancer by reducing cancer promoting activities.

LncRNAs have roles in transcriptional regulation. Members of this class of noncoding RNAs are typically longer than 200 nucleotides, transcribed by RNA polymerase II, and processed similarly to mRNAs. LncRNAs regulate transcription in a variety of ways; they can alter chromatin by directing histone-modifying enzymes to their target loci to induce changes in chromatin, or can regulate transcription directly by interacting with transcription factors and RNA polymerase II. LncRNAs are often key regulators of epigenetic changes that can drive cancer progression, in some cases by aberrant overexpression in other cases by expression during cancer progression.

In some embodiments, N6-methyladenosine (m6A) sites are targeted in lncRNAs disclosed herein in order to regulate lncRNA activity. M6A is a reversible RNA modification. It has been well studied in messenger RNAs (mRNAs), where it can regulate multiple steps of mRNA life cycle, including processing, decay, and translation; however, how m6A regulates lncRNA-mediated processes was unrecognized prior to this disclosure. m6A modification on an RNA is typically recognized by a “reader” protein that binds specifically to methylated adenosine to mediate the functional outcome of m6A deposition. Apart from the YTH family of proteins that directly read m6A (interact with this region), a handful of non-canonical indirect m6A readers have been suggested. For example, in the case of Xist, YTHDC1 recognizes m6A on Xist to mediate repression of the X chromosome; however, how m6A and YTHDC1 contribute to Xist function is unclear. In contrast, m6A on cis-acting chromatin-associated regulatory RNAs leads to a YTHDC1-dependent degradation, preventing transcription of downstream genes.

In some embodiments, modification of methylation sites in the lncRNA HOTAIR interfere with downstream effects of cancer promoting proteins like YTHDC1 or other interactions with HOTAIR to modulate cancer promoting effects thereof. In some embodiments, modification of methylation sites in the lncRNA HOTAIR interfere with YTHDC1's ability to facilitate chromatin localization of HOTAIR. In certain embodiments, modification of methylation sites in the lncRNA HOTAIR interferes with YTHDC1's ability to facilitate chromatin localization of HOTAIR and can affect doubling time (e.g. increases tumor cell doubling time) and affects invasion/metastasis of tumor cells. In some embodiments, constructs, compositions and combination treatments disclosed herein can reduce tumor expansion and/or metastasis.

Human lncRNA HOTAIR is a 2.2 kb (2,158 nucleotide) spliced and polyadenylated RNA transcribed from the HoxC locus. HOTAIR has certain conserved RNA structures but poor sequence conservation across vertebrates. It is known to have a role in promoting cancers across vertebrate species, but the mechanisms of action were not understood in breast cancer until the instant disclosure. Originally identified as a developmental regulator acting in trans to repress expression of the HoxD locus, abnormally high levels of HOTAIR are associated in certain instances with poor survival of cancer patients and increased cancer metastasis in several cancer types, including breast cancer. At its target loci, HOTAIR mediates the induction of H3K27 trimethylation (H3K27me3) by Polycomb Repressive Complex 2 (PRC2), resulting in heterochromatin formation and repression. In a cancer setting or condition, high levels of HOTAIR can misdirect this mechanism to loci that are not typically repressed in the tissue of origin. It is noted that HOTAIR can repress genes even in the absence of PRC2. How HOTAIR accomplishes transcriptional repression and how other pathways, and tumor environments influence HOTAIR function remains elusive but it is noted that HOTAIR plays a role in cancer expansion and progression as well as metastasis of certain tumors.

In certain embodiments, increased levels of HOTAIR and/or YTHDC1 in a subject having cancer of use to assess treatment prospects disclosed herein can include a 10% to a 5 fold increase; a 20% to a 4 fold increase; a 30% to a 3 fold increase; a 40% to a 2 fold increase of HOTAIR and/or YTHDC1 compared to a control subject not having cancer or to a control subject having cancer with low levels of HOTAIR and/or YTHDC1 polynucleotide expression or polypeptide levels. In certain embodiments, a subject having elevated levels of HOTAIR and/or YTHDC1 can be treated by compositions and methods disclosed herein to reduce expansion and/or metastasis of cancer in the subject (e.g., breast cancer).

In some embodiments, the modified methylation sites are N6-methyladenosine (m6A) where adenine is substituted by a different nucleotide that is incapable of being methylated. In accordance with these embodiments, adenine at one or more m6A site can be replaced by uracil, guanine or cytosine to eliminate methylation at the site. In certain embodiments, the substitution is an adenine to uracil substitution. In some embodiments, the HOTAIR methylation site includes, but is not limited to, a substitution at adenine 783 (A783) of SEQ ID. NO: 1 or fragment thereof, or equivalent position thereof. In other embodiments, modified, mutated, synthetically generated or substituted lncRNAs disclosed herein form part of a composition for use in treating a health condition where their respective naturally-occurring lncRNAs have adverse effects. In some embodiments, a composition including modified HOTAIR as disclosed herein can be used to treat a subject with cancer.

In other embodiments, other methylation sites can be blocked in HOTAIR in order to modulate including, one or more adenine nucleotide substitutions at one or more of nucleotide position 48, 102, 143, 215, 620, 655, 722, 936, 1394, 1579, 1663, 1722, 1739 of SEQ ID. NO: 1 or equivalent position thereof of HOTAIR or fragment thereof, and eliminating methylation at the one or more of nucleotide position 48, 102, 143, 215, 620, 655, 722, 936, 1394, 1579, 1663, 1722, or 1739 or equivalent position thereof. In accordance with these embodiments, a synthetic polynucleotide can contain one or more m6A nucleotide substitutions at one or more adenine positions including, but not limited to, 783, and one or more of, 48, 102, 655, 722, 1739 of SEQ ID. NO: 1 or equivalent position thereof. In some embodiments, one or more methylation sites in HOTAIR represented by SEQ. ID. NO. 4 is mutated to reduce or prevent methylation at the site.

In certain embodiments, a synthetic polynucleotide or mutated HOTAIR polynucleotide or fragment thereof can further include a cell targeting agent or moiety. In some embodiments, a cell targeting agent or moiety can include a cancer cell targeting agent or moiety.

In other embodiments, one or more antisense oligonucleotide (ASO) capable of pairing by complementary interaction with HOTAIR (HOX (homeobox) transcript antisense intergenic RNA) is contemplated. In accordance with these embodiments, one or more ASO capable of pairing to one or more methylation sites in HOTAIR is disclosed herein in order to reduce or eliminate methylation at the one or more methylation sites. In one embodiment, one or more ASO that recognizes and pairs with a methylation site in HOTAIR can include, but is not limited to, recognition and pairing by complementarity to adenine position 783 of SEQ ID. NO: 1, fragment thereof or equivalent position thereof and where the one or more ASO blocks methylation of position A783 or equivalent position thereof. In certain embodiment, the one or more ASO is at least six (6) nucleotides in length. In certain embodiments, the ASO can be about 10, about 20, about 30, about 40, about 50, about 60 or greater in length for pairing with HOTAIR. In other embodiments, the one or more ASO is about 10 to about 50 nucleotides in length. In some embodiments, one or more ASOs can include polynucleotide having a thymine (or uracil) that pairs with adenosine at position 783 of SEQ ID. NO: 1 or equivalent position thereof to block methylation at this position. In other embodiments, the one or more ASO further includes a cell targeting agent or moiety. In other embodiments, the ASO can further include at least one chemical modification to the ASO phosphodiester backbone. In yet other embodiments, the at least one chemical modification to the ASO phosphodiester backbone includes, but is not limited to, phosphorothioate DNA, phosphorodiamidate morpholino (PMO), peptide nucleic acid, tricyclo-DNA, ribose substitution 2′-O-methyl (2′-OMe), ribose substitution 2′-O-methoxyethyl (2′-MOE), ribose substitution locked nucleic acid, or any combination thereof.

In certain embodiments an ASO of use to inhibit or silence methylation at the A783 site or equivalent position thereof represented by SEQ ID NO. 1 (or equivalent position and HOTAIR molecule thereof) can include, but is not limited to, the following proposed ASOs. (Of note the ASO start disclosed herein is relative the A783 of SEQ ID. NO. 1 but equivalent positions in HOTAIR variants are contemplated herein). In some embodiments, the ASO is about 5, about 10, about 15, about 20, about 25, about 30, about 35, about 40 or more nucleotides (nts) in length of use to pair with WT HOTAIR or a non-antimorph sequence to reduce or eliminate methylation at A783 or equivalent position thereof and/or designed to pair with and block methylation at other m6A sites disclosed herein. The following ASO are 20 nt (SEQ IDs. NO. 5-24) also numbered 0-19 as indicated. In some embodiments, an ASO of use to pair or associate with WT HOTAIR includes one or more sequence represented by one or more of SEQ ID. NO. 5-24. In other embodiments, an ASO of use to pair or associate with WT HOTAIR includes one or more sequence represented by one or more of SEQ ID. NO. 5, 15 and 24 (0, 10 and 19).

| | |
|---|---|
| 0 | CGCTCAGGTTTTTCCAGCGT SEQ ID. NO. 5 |
| -1 | GCTCAGGTTTTTCCAGCGTT SEQ ID. NO. 6 |
| -2 | CTCAGGTTTTTCCAGCGTTC SEQ ID. NO. 7 |
| -3 | TCAGGTTTTTCCAGCGTTCT SEQ ID. NO. 8 |
| -4 | CAGGTTTTTCCAGCGTTCTG SEQ ID. NO. 9 |
| -5 | AGGTTTTTCCAGCGTTCTCT SEQ ID. NO. 10 |
| -6 | GGTTTTTCCAGCGTTCTCTG SEQ ID. NO. 11 |
| -7 | GTTTTTCCAGCGTTCTCTGG SEQ ID. NO. 12 |
| -8 | TTTTTCCAGCGTTCTCTGGG SEQ ID. NO. 13 |
| -9 | TTTTCCAGCGTTCTCTGGGC SEQ ID. NO. 14 |
| -10 | TTTCCAGCGTTCTCTGGGCG SEQ ID. NO. 15 |
| -11 | TTCCAGCGTTCTCTGGGCGT SEQ ID. NO. 16 |
| -12 | TCCAGCGTTCTCTGGGCGTT SEQ ID. NO. 17 |
| -13 | CCAGCGTTCTCTGGGCGTTC SEQ ID. NO. 18 |
| -14 | CAGCGTTCTCTGGGCGTTCA SEQ ID. NO. 19 |
| -15 | AGCGTTCTCTGGGCGTTCAT SEQ ID. NO. 20 |
| -16 | GCGTTCTCTGGGCGTTCATG SEQ ID. NO. 21 |
| -17 | CGTTCTCTGGGCGTTCATGT SEQ ID. NO. 22 |
| -18 | GTTCTCTGGGCGTTCATGTG SEQ ID. NO. 23 |
| -19 | TTCTCTGGGCGTTCATGTGG SEQ ID. NO. 24 |

In other embodiments, a CRISPR genome editing approach can be used to edit one or more m6A sites in HOTAIR, including but not limited to. editing A783 in HOTAIR. In accordance with these embodiments, a CRISPR genome editing system (e.g., CRISPR/Cas9) can be used to edit A783 to a uracil. In some embodiments, this approach to elicit the HOTAIR antimorph effect is to introduce gene editing tools of the CRISPR system that would target A783 for mutation. A single guide RNA (gRNA) targeting the sequence immediately at A783 would lead to a single cut in the genome and a double-strand break that would be repaired by a non-homologous end joining (NHEJ) mechanism, most-frequently leaving a deletion and/or a mutation at the site of cutting. In another embodiment, a CRISPR approach can include two guide RNAs that cut in a staggered manner, promoting homology-directed repair (HDR) which can be complemented with a synthetic donor single-stranded oligo DNA nucleotide (ssODN) donor for “swapping” of the wild-type HOTAIR sequence with the exact A783U sequence that elicits an anti-morph effect. In other embodiments, other m6A sites in HOTAIR can be edited to improve stability of an antimorph HOTAIR of use to treat cancer in a subject.

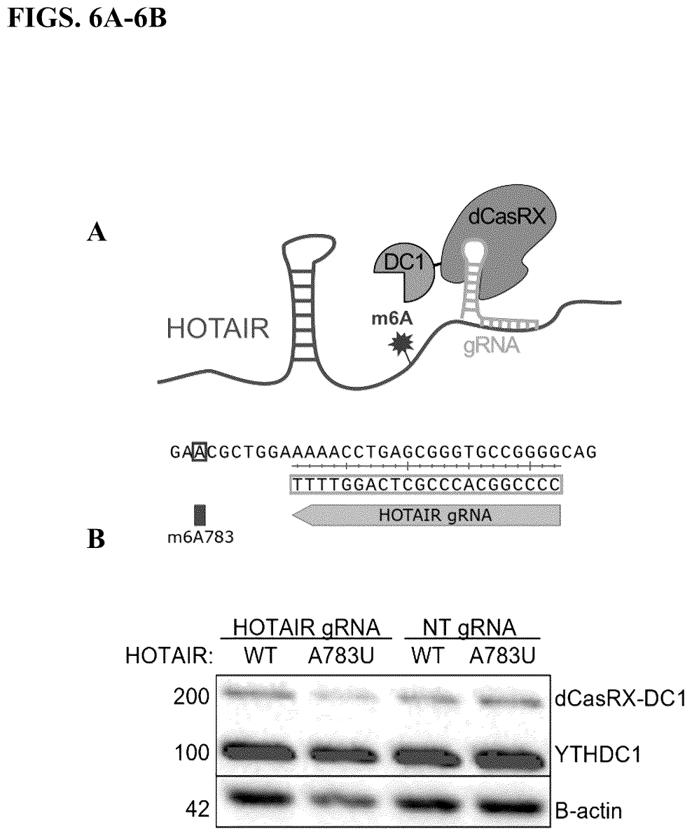

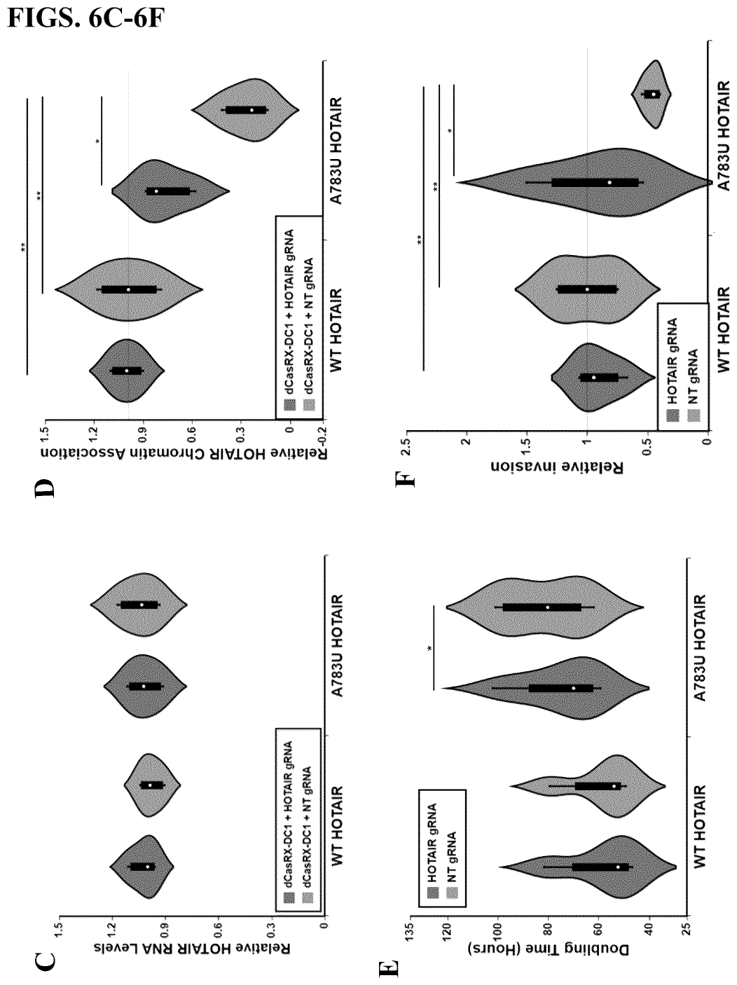

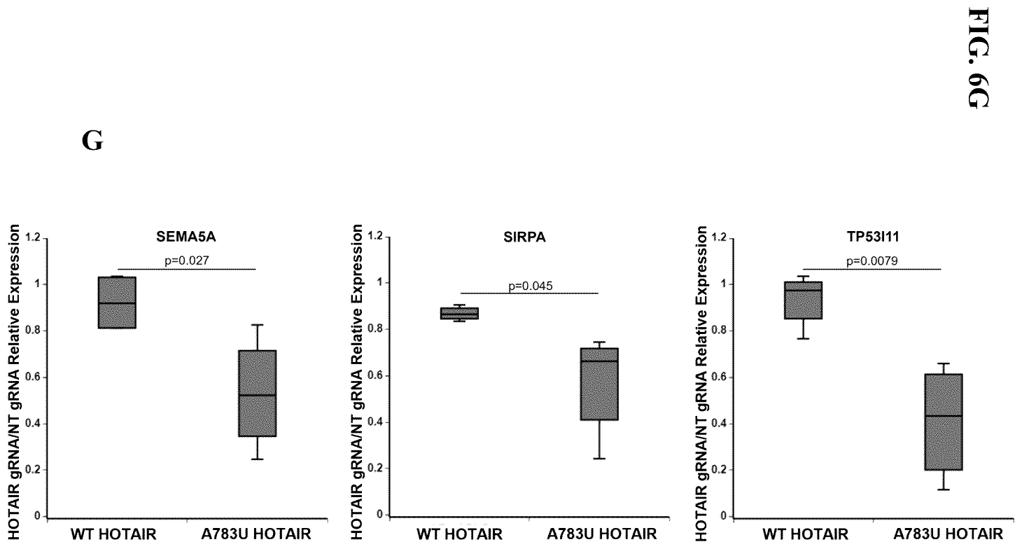

In other embodiments, a modified CRISPR system can be used to recruit YTHDC1 effector protein to a mutated form of HOTAIR in order to interfere with chromatin association of HOTAIR. (See for example FIGS. 6A-6G) It is contemplated herein that a catalytically inactive RNA-targeting Cas protein (dCasRX) can be used to recruit effector protein such as YTHDC1 or other effector protein to mutated RNA including a mutated A783 HOTAIR RNA via use of a guide RNA thereby lowering HOTAIR chromatin association. In some embodiments, plasmids encoding these molecules can be delivered to a cell to express one or more constructs.

In other embodiments, compositions disclosed herein include one or more mutated HOTAIR and/or one or more ASO. In other embodiments, compositions disclosed herein are pharmaceutical compositions and further include a pharmaceutically acceptable excipient or agent. In some embodiments, a composition disclosed herein can include a buffer for stabilizing one or more synthetic polynucleotide having one or more substitutions or the anti-sense oligonucleotide. In some embodiments, a composition disclosed herein can include a buffer for stabilizing one or more synthetic polynucleotide having one or more substitutions or the anti-sense oligonucleotide. In some embodiments, a mutated, substituted, or modified HOTAIR polynucleotide (e.g., RNA) and/or one or more ASO to HOTAIR can be used to treat a subject having cancer (e.g., breast cancer or breast cancer having elevated HOTAIR expression and/or YTHDC1 expression).

In some embodiments, a mutated, substituted, or modified HOTAIR polynucleotide (e.g., RNA) and/or one or more ASO to HOTAIR can be stored in a standard buffer for prolonged periods and later use. In certain embodiments, a buffer can include PBS, HEPES, Tris, Tris-HCl, or other suitable buffer with or without contaminant reducing agents. In other embodiments, buffer pH can be about 6.5 to about 8.5 or about 7.0 to about 8.0 or about 7.2-7.6. In yet other embodiments, other agents can be used to supplement the buffer and enhance stabilization of these molecules such as salt, EDTA, poloxamers, disaccharides, or polymers for stabilization during storage processes or the like.

In yet other embodiments, methods for treating a condition having aberrant expression of HOTAIR are contemplated. In some embodiment, the condition includes cancer. In certain embodiments, methods include, but are not limited to, administering a composition including one or more mutant or ASO disclosed herein to the subject and treating the cancer in the subject. In other embodiments, the cancer includes a solid tumor that is currently or is suspected of expressing or overexpressing HOTAIR. In certain embodiments, the cancer includes, but is not limited to, breast, endometrial, prostate, pancreatic, glioma, lung (e.g. small cell lung), liver, stomach, colon, intestinal, other gastric cancer, ovarian or any combination thereof or any metastasizing tumor thereof.

In certain embodiments, the composition includes constructs having at least an A783U mutation in HOTAIR represented by SEQ. ID. NO.1, or equivalent position of HOTAIR thereof or fragment thereof. where the composition induces one or more of reduces HOTAIR cancer-promoting activity or activities, reduces HOTAIR expression, reduces HOTAIR chromatin association, reduces YTHDC1 interactions with HOTAIR, and induces cancer suppression in the subject to treat the cancer in the subject. In certain embodiments, the cancer being treated includes breast cancer. In other embodiments, a subject being treated for cancer using a composition containing mutant HOTAIR and/or one or more ASOs can further be treated with one or more standard cancer treatments. In accordance with these embodiments, the one or more standard treatments can include, but are not limited to, radiation, surgery, chemotherapy, immunotherapy, hormone therapy, stem cell therapy, bone marrow transplantation, or other method for treating cancer. In certain embodiments, the one or more standard treatments for treating cancer of radiation, surgery, chemotherapy, immunotherapy, hormone therapy, stem cell therapy, bone marrow transplantation, or other method for treating cancer can be performed before, during or after treating the subject with the composition contemplated herein. In other embodiments, the standard treatment is radiation which follows treatment with one or more composition containing a mutant HOTAIR and/or one or more ASOs directed to bind one or more methylation site when pairing with HOTAIR in the subject.

In some embodiments, administering compositions disclosed herein can include administering a composition intravenously, by continuous infusion over a predetermined period of time, subcutaneously, intraocularly, topically, intradermally, intranasally, directly into the tumor or other mode. In yet other embodiments, compositions can be administered to a subject daily, every other day, bi-weekly, weekly, bi-monthly, monthly, every other month or other time period determined by a healthcare provider. In some embodiments, a subject can be treated by a regimen determined by a health professional with a combination composition disclosed herein or separately administered compositions of mutated HOTAIR and ASOs alone or in combination with surgery and/or radiation to treat cancer in the subject.

In certain embodiments, a subject having cancer or suspected of developing cancer can be assessed for levels or concentration of HOTAIR and/or YTHDC1 in order to develop a treatment regimen using compositions and methods disclosed herein. In certain embodiments, combination therapies can include modifying m6A methylation sites in a polynucleotide construct of HOTAIR of use to administer to a subject in combination with one or more ASO and/or in combination with an antibody, small molecule or other agent that blocks YTHDC1 from binding to at least A783 of HOTAIR and thereby treating or preventing cancer or cancer metastasis in the subject. In some embodiments, viral vectors (e.g. AAV) known in the art or modified viral vectors known in the art can be used to deliver CRISPR/Cas9 constructs contemplated herein (e.g. to modify A783 in HOTAIR to reduce or prevent methylation). In other embodiments, eraseRNAs can be used to target at least one m6A site such as A783 of HOTAIR to reduce chromatin association and cancer promoting activities of HOTAIR in cancer (e.g., breast cancer). In yet other embodiments, combination methods for treating a subject having cancer with elevated levels of HOTAIR can be treated with compositions to reduce or block methylation of at least A783 and methods and compositions for reducing or targeting the methylating enzyme thereof. In certain embodiments, treatments disclosed herein can be alternating treatments or periodic treatments where levels of HOTAIR can be measured and further treatment assessed.

Other embodiments disclosed herein concern kits for storing or transporting or storing and transporting of at least one of synthetic HOTAIR polynucleotides having one or more substitution, mutation or modification to reduce or prevent adenosine methylation or one or more anti-sense oligonucleotide (ASO) that pairs with one or more HOTAIR methylation sites (e.g. to block or reduce methylation of the one or more site) and at least one container. Kits can further include devices, buffers and other components. In certain embodiments, kits are provided for use in treating or reducing the size of, or reducing expansion of a tumor.

In certain embodiments, methods and compositions provided herein can include a vector containing any one of the polynucleotides disclosed herein. In some embodiments, a vector for use herein can be a viral vector. As used herein, the term “viral vector” can refer to a nucleic acid vector construct that includes at least one element of viral origin and has the capacity to be packaged into a viral vector particle, and encodes at least an exogenous polynucleotide. In certain embodiments, the vector and/or particle can be utilized for the purpose of transferring any nucleic acids into cells either in vitro or in vivo. Numerous viral vectors are known in the art. The term virion can refer to a single infective viral particle. “Viral vector”, “viral vector particle” and “viral particle” also refer to a complete virus particle with its DNA or RNA core and protein coat as it exists outside the cell. Non-limiting examples of viral vectors for use herein can include adenoviruses, adeno-associated viruses (AAV), herpesviruses, retroviruses, lentiviruses, integrase defective lentiviruses (IDLV), and the like. In some embodiments, a viral vector disclosed herein can be a lentiviral vector. Examples of lentiviruses include, but are not limited to, human lentiviruses such as HIV (in particular HIV-1 or HIV-2), simian immunodeficiency virus (SIV), equine infectious anemia virus (EIAV), feline immunodeficiency virus (FIV), Caprine Arthritis Encephalitis Virus (CAEV), visna and progressive pneumonia viruses of sheep, baboon pseudotype viruses, bovine immunodeficiency virus (BIV), and the like.

In some embodiments, polynucleotides and/or vectors described herein can be prepared by conventional recombinant technology known to one of skill in the art. In other embodiments, polynucleotides and/or vectors described herein can be prepared by a gene editing methods known in the art (e.g., by CRISPR).

In certain embodiments, methods provided herein can include generating a cell to express any of the polynucleotides and/or vectors described herein. In some embodiments, cells for use herein can be one or more immune cells. As used herein an “immune cell” can refer to a cell of the immune system. Immune cells can be categorized as lymphocytes, neutrophils, granulocytes, mast cells, monocytes/macrophages, and dendritic cells. In some embodiments, cells for use herein can be one or more lymphocytes.

In some embodiments, methods and compositions provided herein can include a cell having a vector and/or a polynucleotide encoding mutant HOTAIR contemplated herein. In some embodiments, methods herein can include introducing any vector and/or a polynucleotide disclosed herein into any system known in the art for generating polynucleotides and/or ASOs disclosed herein.

In certain embodiments, pharmaceutical compositions are contemplated. In accordance with these embodiments, pharmaceutical compositions can include one or more of the polynucleotides, polypeptides, vectors-containing constructs disclosed herein, and/or cells containing constructs or expression constructs described herein. In some embodiments, pharmaceutical compositions herein can include one or more of the polynucleotides, vectors-containing constructs disclosed herein, and/or cells containing constructs or expression constructs described herein and at least one pharmaceutically acceptable excipient or carrier. As used herein, the term “pharmaceutically acceptable” refers to those compounds, materials, compositions, and/or dosage forms which are, within the scope of sound medical judgment, suitable for use in contact with the tissues, organs, and/or bodily fluids of a subject without excessive toxicity, irritation, allergic response, or other problems or complications commensurate with a reasonable benefit/risk ratio. As used herein, the term “pharmaceutically acceptable carrier” can refer to solvents, dispersion media, coatings, antibacterial agents, antifungal agents, isotonic and absorption delaying agents, or the like that are physiologically compatible. Pharmaceutically acceptable carriers suitable for use herein, include, but are not limited to, buffers that are well known in the art, and can be phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

In some embodiments, pharmaceutical compositions for use herein can be formulated for parenteral administration, such as intravenous, intracerebroventricular injection, intra-cisterna magna injection, intra-parenchymal injection, intra-renal, intradermal, subcutaneous, direct introduction to a tumor or a combination thereof. In some embodiments, pharmaceutical compositions for use herein can be formulated for local delivery to one or more tumors. In some embodiments, pharmaceutical compositions for use herein be formulated for parenteral administration can include pharmaceutically acceptable carriers including sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. In some embodiments, pharmaceutical compositions for use herein can further include additional agents, for example preservatives, buffers, tonicity agents, antioxidants and stabilizers, nonionic wetting or clarifying agents, viscosity-increasing agents, and the like. In some embodiments, pharmaceutical compositions described herein can be packaged in single unit dosages or in multi-dosage forms.

In some embodiments, formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. In accordance with some embodiments herein, aqueous solutions can be suitably buffered (preferably to a pH of from 3 to 9). The preparation of suitable parenteral formulations for use herein under sterile conditions can be readily accomplished by standard pharmaceutical techniques well known to those skilled in the art.

In some embodiments, pharmaceutical compositions herein can further include one or more pharmaceutically acceptable salts. Non-limiting examples of pharmaceutically acceptable salts include acid addition salts (formed from a free amino group of a polypeptide with an inorganic acid, or an organic acid. In some embodiments, the salt formed with the free carboxyl groups is derived from an inorganic base, or an organic base. In some embodiments, any of the pharmaceutical compositions herein can be used in therapeutic applications, for example, cancer treatment in human patients, which are also disclosed herein.

In certain embodiments, methods of treating or ameliorating cancer, a tumor, or a combination thereof in a subject are disclosed. In some embodiments, methods of treating or ameliorating a solid tumor in a subject include, but are not limited to, administration of an effective amount of any the polynucleotides, ASOs, vectors-containing constructs disclosed herein, and/or cells containing constructs or expression constructs and/or pharmaceutical compositions containing these agents thereof described herein. “An effective amount” as used herein refers to a dose that is sufficient to confer a therapeutic effect on a subject having or suspected of having cancer, a tumor, or any combination thereof and further treating the cancer. In certain embodiments, a therapeutic effect for a subject having or suspected of having a tumor can include reducing the symptoms or consequences of the cancer, such as reducing expansion of, shrinking of a tumor, killing tumor cells, preventing the occurrence of metastases from a primary tumor, reducing the number of tumor cells of a tumor, primary tumor and/or a metastatic tumor, inhibiting the growth of tumor cells of a primary tumor and/or a metastatic tumor, eliminating tumor cells in a subject by killing the cells or preventing propagation or expansion of the solid tumor cells and the like.

In some embodiments, a subject to any of the methods herein can be any subject for whom treatment or therapy is desired. In some embodiments, a subject can have or can be suspected of having cancer, a tumor, or any combination thereof. In other embodiments, a subject can have or can be suspected of having one or more primary tumors, one or more metastatic tumors such as solid tumors or any combination thereof. In other embodiments, a subject can be a mammal. In some embodiments, a subject can be a human patient. In yet other embodiments, a human patient such as an adult, child, adolescent, toddler, young adult or infant or fetus who is in need of the methods herein can be identified by routine medical examination, e.g., laboratory tests, biopsy, magnetic resonance imaging (MRI) scans, ultrasound exams, and the like.

In some embodiments, a subject to be treated by the methods described herein can be a human patient having, suspected of having, or a risk for developing cancer or having cancer spread such as through metastasis. In some embodiments, a subject to be treated by the methods described herein can have or be suspected of developing breast cancer or other cancer such as other solid tumor or tumor expressing HOTAIR.

In some embodiments, after administration of any the polynucleotides, vectors, cells and/or pharmaceutical compositions (e.g., mutant HOTAIR polynucleotide) described herein, a subject can be treated by radiation therapy. In some embodiments, a subject can be treated by radiation therapy to improve response to radiation therapy after dampening the effects of HOTAIR on tumor promotion. In some embodiments, a subject can be treated by radiation therapy within minutes, to within hours after administration of compositions disclosed herein or within about 6 hours, about 12 hours, about 24 hours, about 2 days, about 3 days, about 4 days, about 5 days, about 6 days, about 1 week, or about 2 weeks after administration of compositions disclosed herein. In some embodiments, a subject can be treated by radiation therapy using ionizing radiation. In some embodiments, a subject can be treated by radiation therapy delivered by a linear accelerator. In some embodiments, a subject can be treated by radiation therapy delivered directly to a tumor. In some embodiments, radiation therapy can be delivered directly to a tumor at a dose of radiation ranging from about 2 Gy to about 50 Gy (e.g., about 2, about 5, about 10, about 20, about 30, about 40, about 50 Gy).

In certain embodiments, surgical tumor remedies can be used before, during or after compositions disclosed herein are used to reduce HOTAIR cancer promoting activities. In some embodiments, surgery can be used to remove a breast cancer mass and at the same time, before and or after, compositions disclosed herein can be used to treat the subject to reduce HOTAIR cancer promoting activities.

In some embodiments, a subject treated with any of the methods herein can have completed an additional therapeutic regimen, be receiving an additional therapeutic regimen, or can receive an additional therapeutic regimen following treatment herein. In some embodiments, an additional therapeutic regimen for use herein can include administering a chemotherapeutic agent. In some embodiments, a chemotherapeutic agent can be a cell cycle inhibitor. As used herein “cell cycle inhibitor” can include a chemotherapeutic agent that inhibits or prevents the division and/or replication of cells. In some embodiments, a cell cycle inhibitor can include a chemotherapeutic agent such as Doxorubicin, Melphlan, Roscovitine, Mitomycin C, Hydroxyurea, 5-Fluorouracil, Cisplatin, Ara-C, Etoposide, Gemcitabine, Bortezomib, Sunitinib, Sorafenib, Sodium Valproate, a HDAC Inhibitor, or Dacarbazine. More examples of additional chemotherapeutic agents include, but are not limited to HDAC inhibitors such as FR01228, Trichostatin A, SAHA and/or PDX101. In some embodiments, the cell cycle inhibitor is a DNA synthesis inhibitor. As used herein, a “DNA synthesis inhibitor” can include a chemotherapeutic agent that inhibits or prevents the synthesis of DNA by a cancer cell. Examples of DNA synthesis inhibitors include but are not limited to AraC (cytarabine), 6-mercaptopurine, 6-thioguanine, 5-fluorouracil, capecitabine, floxuridine, gemcitabine, decitabine, vidaza (aza), fludarabine, nelarabine, cladribine, clofarabine, pentostatin, thiarabine, troxacitabine, sapacitabine or forodesine. More examples of additional chemotherapeutic agents include, but are not limited to, FLT3 inhibitors such as Semexanib (SCT5416), Sunitinib (SU 11248), Midostaurin (PKC412), Lestautinib (CEP-701), Tandutinib (MLN518), CHIR-258, Sorafenib (BAY-43-9006) and/or KW-2449. More non-limiting examples of additional chemotherapeutic agents include farnesyltransferase inhibitors such as tipifarnib (RI 15777, Zarnestra), lonafarnib (SCH66336, Sarasar™) and/or BMS-214662. More examples of additional chemotherapeutic agents include, but are not limited to, topoisomerase II inhibitors such as the epipodophyllotoxins etoposide, teniposide, anthracyclines doxorubicin and/or 4-epi-doxorubicin. More non-limiting examples of additional chemotherapeutic agents include P-glycoprotein modulators such as zosuquidar trihydrochloride (Z.3HCL), vanadate, or verapamil. More non-limiting examples of additional chemotherapeutic agents include hypomethylating agents such as 5-aza-cytidine or 2′ deoxyazacitidine.

In some embodiments, kits are contemplated of use to generate constructs disclosed herein. In other embodiments, kits include therapeutic compositions disclosed herein for storage, transport and use. In other embodiments, a kit can include any of the polynucleotides. polypeptides, vectors-containing constructs disclosed herein, and/or cells containing constructs or expression constructs or pharmaceutically acceptable formulation disclosed herein. In some embodiments, a kit can further include one or more reagents for storing constructs disclosed herein. For example, a kit can include media, surfactants, salts, or other agents for storing constructs disclosed herein. In some embodiments, a kit can further include an insert with instruction for making or using constructs or ASOs disclosed herein.

Kits disclosed herein include suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition can be a mutant HOTAIR construct population (e.g., a polynucleotide therapeutic) and/or HOTAIR ASOs described herein.

Kits can optionally provide additional components such as buffers and interpretive information. Kits can include a container and a label or package insert(s) on or associated with the container. In some embodiments, the invention provides articles of manufacture including contents of the kits described above.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

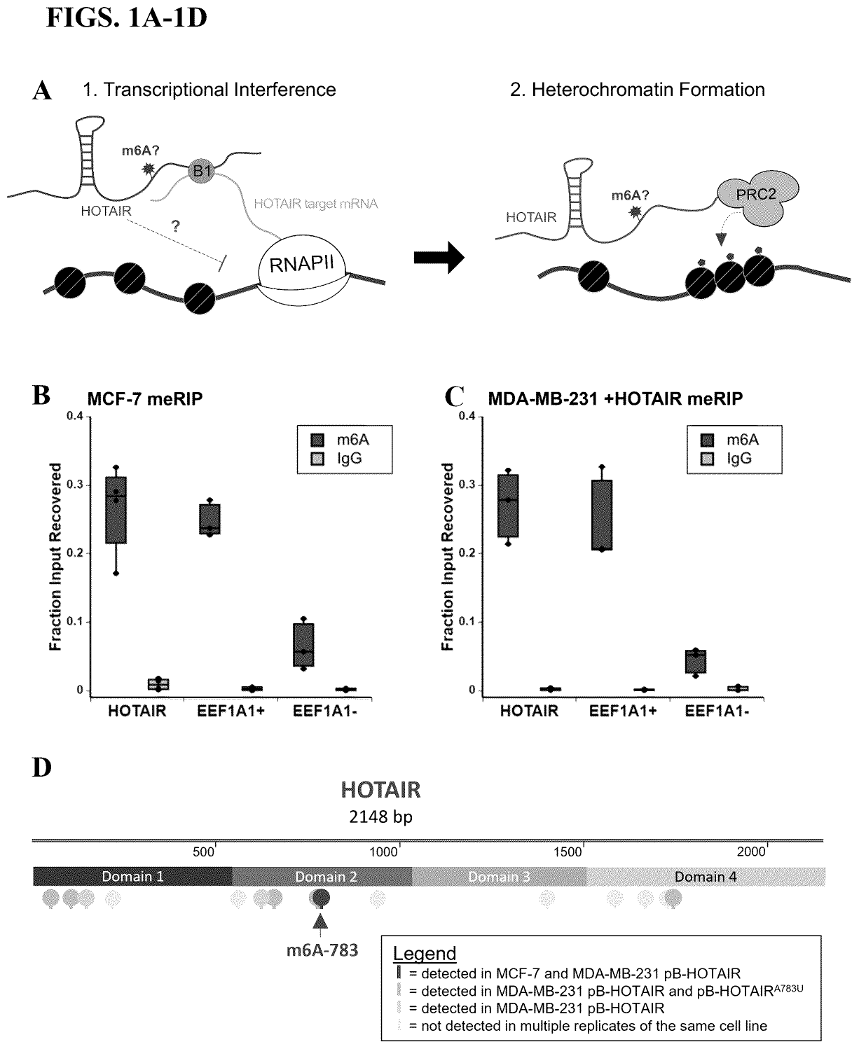

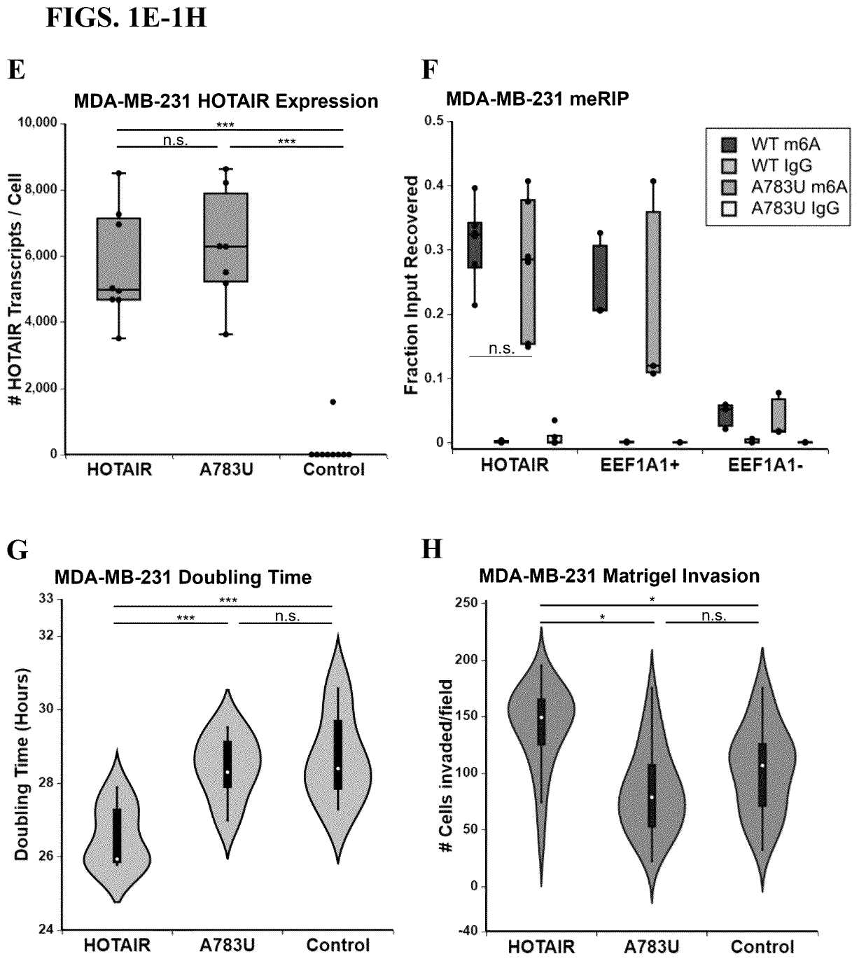

In one exemplary method, N6-methyladenosine (m6A) sites in HOTAIR were investigated for effect on HOTAIR. It is understood that these types of modifications in other RNAs play important roles in normal biology and cancer biology. Knowledge of methylations on long noncoding RNAs (lncRNAs) remains limited. The following examples illustrate the importance of methylation in the lncRNAs, HOTAIR. For example, as illustrated in FIG. 1A, HOTAIR was initially recruited to its target loci via RNA-RNA interactions with its mRNA targets which was mediated by hnRNP B1. HOTAIR association with chromatin induced transcriptional interference via an unknown mechanism, promoting heterochromatin formation by PRC2 through H3K27me3. The examples described herein further investigate the role of m6A on HOTAIR.

In one example, it was investigated whether m6A regulated certain functions of human HOTAIR lncRNAs. In these examples, HOTAIR's role in multiple pro-tumor activities was investigated. For example, phenotypes in triple-negative breast cancer (TNBC) cells were examined. Surprisingly, at least 8 individual m6A sites within HOTAIR, were identified, with a single site (A783) consistently methylated. It was demonstrated herein that mutation of A783 impaired cellular proliferation and invasion in HOTAIR-overexpressing triple negative breast cancer (TNBC) cells. m6A at A783 regulated HOTAIR's ability to localize to chromatin and orchestrate cancer-associated gene expression changes. In one example, an A783U mutant of HOTAIR led to effects opposite to wild-type HOTAIR, demonstrating a surprising antimorphic behavior. These exemplary results demonstrate how modification of at least a single mA6 location in a lncRNA elicited a distinct gene regulation mechanism and drive disease-associated phenotypic changes of use in the treatment of health conditions adversely affected by these structures.

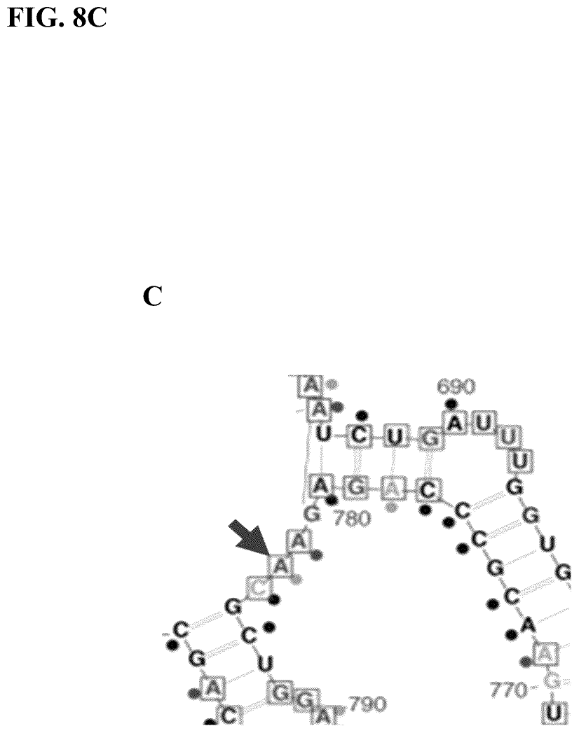

In certain exemplary methods, m6A methylation of HOTAIR was evaluated in exemplary cancer cells. Using the CVm6A database, 3 m6A peaks in HOTAIR in Hela cells were found, although the enrichment score for these sites was low (FIG. 8A). m6A RNA immunoprecipitation (meRIP) qRT-PCR was performed in MCF-7 cells (an exemplary breast cancer cell line), which express low levels of endogenous HOTAIR-˜ 10-fold higher expression than the parent MDA-MB-231 TNBC cell line. A significant concentration of HOTAIR was recovered upon immunoprecipitation with the anti-m6A antibody (26.2%, p=0.006), similar to an m6A modified region on the positive control region of EEF1A1, and consistently higher than a distal region of EEF1A1 that is not m6A modified (See for example, FIG. 1B).

In another exemplary method, m6A modification of HOTAIR was confirmed in a stable MDA-MB-231 cell line with transgenic overexpression of HOTAIR (FIG. 1C). It was observed that overexpression of HOTAIR resulted in 10³-10⁴ copies of HOTAIR per cell and mediated increased cancer aggressiveness and proliferation of MDA-MB-231 cells (FIGS. 1E-1G). meRIP in MDA-MB-231 cells expressing transgenic HOTAIR resulted in significant HOTAIR recovery (27.1%, p=0.001) (FIG. 1B). These exemplary results demonstrate that HOTAIR and the HOTAIR mutants were capable of being over expressed in two distinct breast cancer contexts.

To identify single nucleotide sites of m6A modification, a modified of m6A eCLIP protocol (FIG. 8B) on polyA-selected RNA from MCF-7 and MDA-MB-231 breast cancer cells (data found in Table 1) was used. In MCF-7 cells, at least one m6A site was identified within the HOTAIR transcript at adenosine position 783 (FIG. 1D) as being significant. m6A at adenosine 783 in MDA-MB-231 cells with transgenic HOTAIR was consistently detected with high confidence, along with 7 other sites of likely importance using our multi-replicate consensus approach (see Tables 1 and 2). Interestingly, A783 occurred within a non-canonical ‘GAACG’ sequence located in an unstructured region of the HOTAIR secondary structure (FIG. 8C).

Table 1 provides a list of HOTAIR m6A sites identified by experiment with data provided herein. Each column represents a single experiment with the cell line noted. Each row is an m6A site detected within HOTAIR and includes Nt #(location within HOTAIR transcript) and chromosome position. Each X represents an m6A site detected in HOTAIR in each experiment. X's represent high confidence m6A sites (that showed 3+C->T mutations following the m6A site in ≥5% of reads), m6A sites in bold were included in the 6× HOTAIR mutant, while the remainder of sites (not including Nt 557) were included in the 14× HOTAIR mutant. m6A site 783 is shown as underlined.

TABLE 1

| | Experiments | | | | | | | | | | |
|---|---|---|---|---|---|---|---|---|---|---|---|
| | | | | | | | MDA-MB- | MDA-MB- | MDA-MB- | | 293 |
| | | | | MDA-MB- | MDA-MB- | MDA-MB- | 231 pB- | 231 pB- | 231 pB- | MDA-MB- | HOT- |
| | | | | 231 pB- | 231 pB- | 231 pB- | HOTAI | HOTAI | HOTAI | 231 pB- | AIR- |
| HOTAIR | MCF-7 | MCF-7 | MCF-7 | HOTA IR | HOTA IR | HOTA IR | R^{A783U} | R^{A783U} | R^{A783U} | Anti- | Luc |
| m6A sites | rep1 | rep2 | rep3 | rep1 | rep2 | rep3 | rep 1 | rep2 | rep3 | Luc | dEED |

| | | | | | | | | | | |
|---|---|---|---|---|---|---|---|---|---|---|
| Nt 48/ | | | X | X | | X | x | X | X | x |
| 54362413 | | | | | | | | | | |
| Nt 102/ | | | X | X | | x | * | * | | x |
| 54361137 | | | | | | | | | | |
| Nt 143/ | | | * | * | | | | | | x |
| 54361096 | | | | | | | | | | |
| Nt 215/ | | | * | | | x | | | | x |
| 54360133 | | | | | | | | | | |
| Nt 557/ | | | * | | | | x | | | |
| 54357821 | | | | | | | | | | |
| Nt 620/ | | | X | X | * | | x | | | * |
| 54357758 | | | | | | | | | | |
| Nt 655/ | | | X | X | X | X | | * | X | x |
| 54357723 | | | | | | | | | | |
| Nt 772/ | | | x | * | x | x | X | x | X | x |
| 54357606 | | | | | | | | | | |
| Nt 783/ | X | * | X | X | X | | | | X | X |
| 54357595 | | | | | | | | | | |
| Nt 936/ | | | * | | | x | | | | |
| 54357442 | | | | | | | | | | |
| Nt 1394/ | | | x | | | * | | | | x |
| 54356983 | | | | | | | | | | |
| Nt 1579/ | | | X | | | | | | | * |
| 54356798 | | | | | | | | | | |
| Nt 1663/ | | | x | | | | | | * | |
| 54356714 | | | | | | | | | | |
| Nt 1722/ | | | * | | | | * | | | x |
| 54356655 | | | | | | | | | | |
| Nt 1739/ | | | x | X | | X | * | | | x |
| 54356638 | | | | | | | | | | |

Table 2 provides multiple replicate consensus list of exemplary m6A sites in HOTAIR-expressing breast cancer cell lines. X indicates an m6A site detected in 2+ replicates in the cell line noted.

TABLE 2

| | | MDA-MB-231 pB- | MDA-MB-231 pB- |
|---|---|---|---|
| HOTAIR m6A sites | MCF-7 | HOTAIR | HOTAIR^{A783U} |
| Nt 48/54362413 | | X | X |
| Nt 102/54361137 | | X | X |
| Nt 620/54357758 | | X | |
| Nt 655/54357723 | | X | X |
| Nt 772/54357606 | | X | X |
| Nt 783/54357595 | X | X | |
| Nt 1739/54356638 | | X | X |

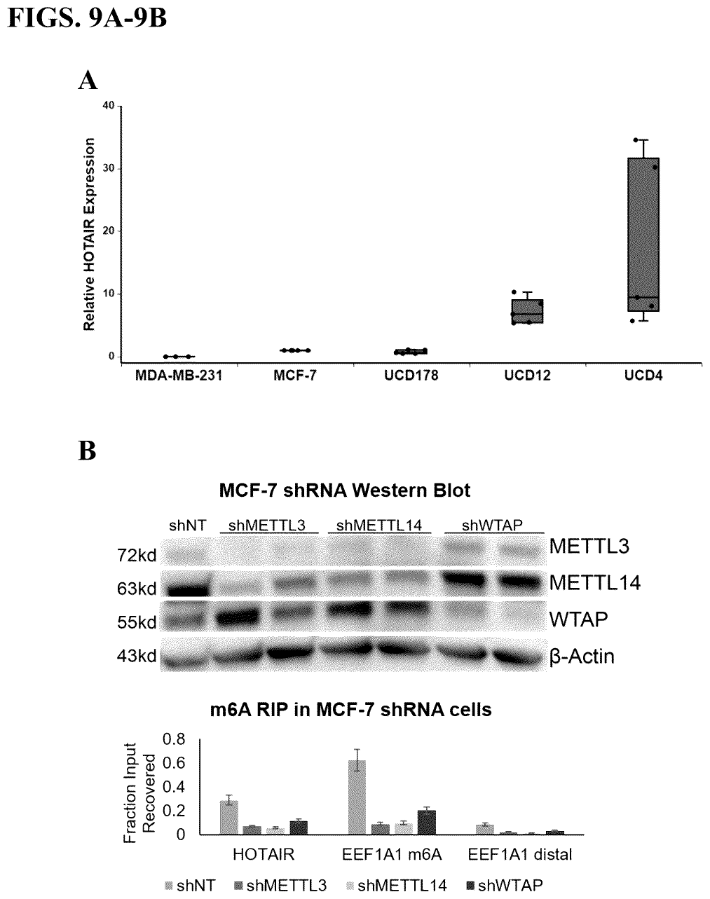

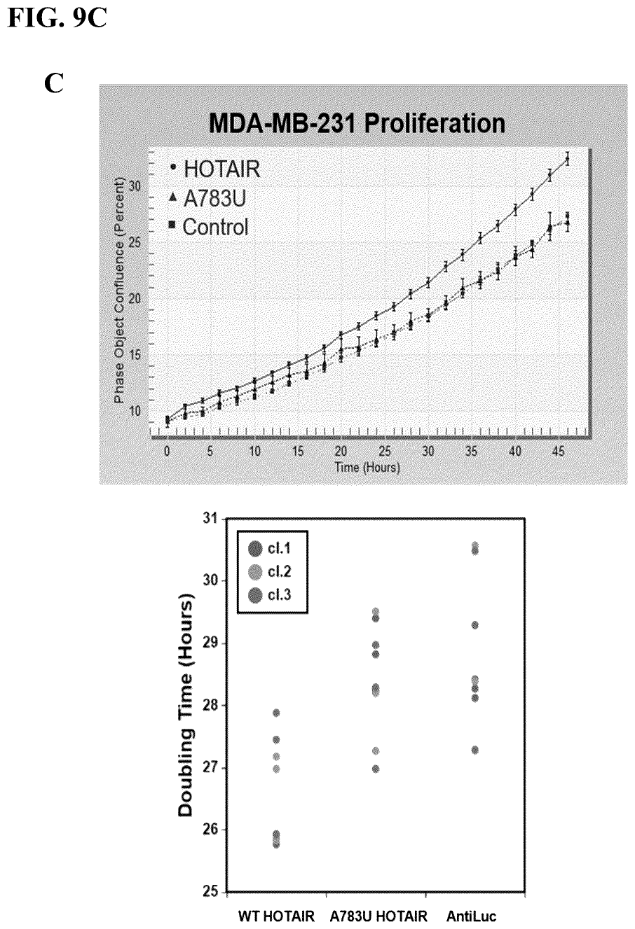

Table 3 represents m6A sites identified in indicated cell lines derived from multiple patient-derived xenografts using meCLIP, providing evidence for m6A at position 783 in recent patient samples expressing HOTAIR (FIG. 9A).

TABLE 3

| | | Raw Reads | Transcripts per |
|---|---|---|---|
| Cell Line | m6A783 | (Conversions) | million (reads)³³ |
| UCD4 | Yes | 66 (6) | 6.7 (85) |
| UCD12 (REP1) | No | 38 (0) | 2.9 (48) |
| UCD12 (REP2) | No | 33 (1) | 2.9 (48) |
| UCD178 (REP1) | No | 16 (0) | 0.4 (8) |
| UCD178 (REP2) | No | 16 (0) | 0.4 (8) |

In another exemplary method, whether HOTAIR was m6A modified by the canonical m6A methyltransferase METTL3/14 complex was tested by using shRNA to deplete METTL3, METTL14, and the adaptor protein WTAP in MCF-7 cells (FIG. 9B). A ˜3 to 5-fold reduced recovery of HOTAIR in methyltransferase-depleted cells relative to non-targeting controls was observed (p=0.0063). Together, these results indicated that m6A methylation of HOTAIR is dependent on the METTL3/14 complex.

Given that nucleotide A783 was consistently methylated within HOTAIR in these exemplary m6A mapping experiments, in both endogenous and overexpressed contexts, it was further analyzed whether this modification had any consequences to HOTAIR function. To directly test the functional role of A783, the adenosine was mutated to uracil at this position (HOTAIR^{A783U}). Various m6A sites in MDA-MB-231 cells overexpressing the HOTAIR^{A783U} mutant were then mapped as provided in Table 1. Both wild-type (WT) and the mutant form of HOTAIR were expressed at similar levels, with approximately 5,000 transcripts per cell (FIG. 1D), resembling the high levels of HOTAIR observed in samples from cancer patients. While the CLIP-based m6A signature was no longer detected at adenosine 783 when this site was mutated to uracil, m6A modification was detected at five of the seven other multi-replicate consensus sites (Tables 2 and 3, FIG. 1E). Nucleotides 143 and 620 were no longer detected with multi-replicate consensus confidence as m6A in the A783U mutant, though m6A143 was only detected in WT HOTAIR at the lowest confidence category and m6A620 was identified in one A783U mutant replicate (Table 2). It is possible that methylation at A783 was required for one or both m6A events to occur. However, meRIP on MDA-MB-231 cells expressing HOTAIR^{A783U} resulted in significant HOTAIR recovery (27.6%, p=0.002 vs IgG), similar to WT HOTAIR-expressing cells (31.2%, p=6.5e-5 vs. IgG) (0.9-fold change, p=0.34) (FIG. 1F), suggesting that HOTAIR^{A783U} maintained m6A modification at other sites in HOTAIR.

In another exemplary method, to determine effects of the A783U mutation on HOTAIR-mediated breast cancer cell growth, doubling time of MDA-MB-231 cells expressing WT and A783U mutant HOTAIR was measured. As described above, HOTAIR and the HOTAIR^{A783U} mutant were overexpressed in MDA-MB-231 cells with overexpression of an antisense sequence of luciferase mRNA (Anti-Luc) used as a negative control. Cell proliferation assays were then performed by plating 5,000 cells in a 96-well dish and analyzing confluency every 2 hours over a period of 48 hours (FIG. 9C). It was observed that MDA-MB-231 cells overexpressing WT HOTAIR had a shorter doubling time (˜26 hours) than cells overexpressing Anti-Luc (˜28.5 hours, p<0.001) (FIG. 1G). Surprisingly, the single nucleotide mutation of A783U in HOTAIR abolished its ability to enhance MDA-MB-231 cell proliferation; cells expressing HOTAIR^{A783U} had a longer doubling time than those expressing WT HOTAIR (˜28.6 hours, p=0.004) and grew similarly to cells containing the Anti-Luc control. To examine the role of A783 of HOTAIR in mediating breast cancer cell invasion, the same MDA-MB-231 cell lines were plated in a Matrigel invasion assay. While overexpression of WT HOTAIR induced a significant increase in number of cells invaded compared to the Anti-Luc control (p=0.038), overexpression of A783U HOTAIR did not lead to an increase in invasion compared to the Anti-Luc control (p=0.22) and resulted in significantly less cells invaded compared to overexpression of WT HOTAIR (p=0.012) (FIG. 1H). Altogether, these surprising results indicated that m6A modification of adenosine 783 in HOTAIR is one key site for mediating the increased aggressiveness of TNBC that is promoted in contexts where the lncRNA is overexpressed.

### Example 2

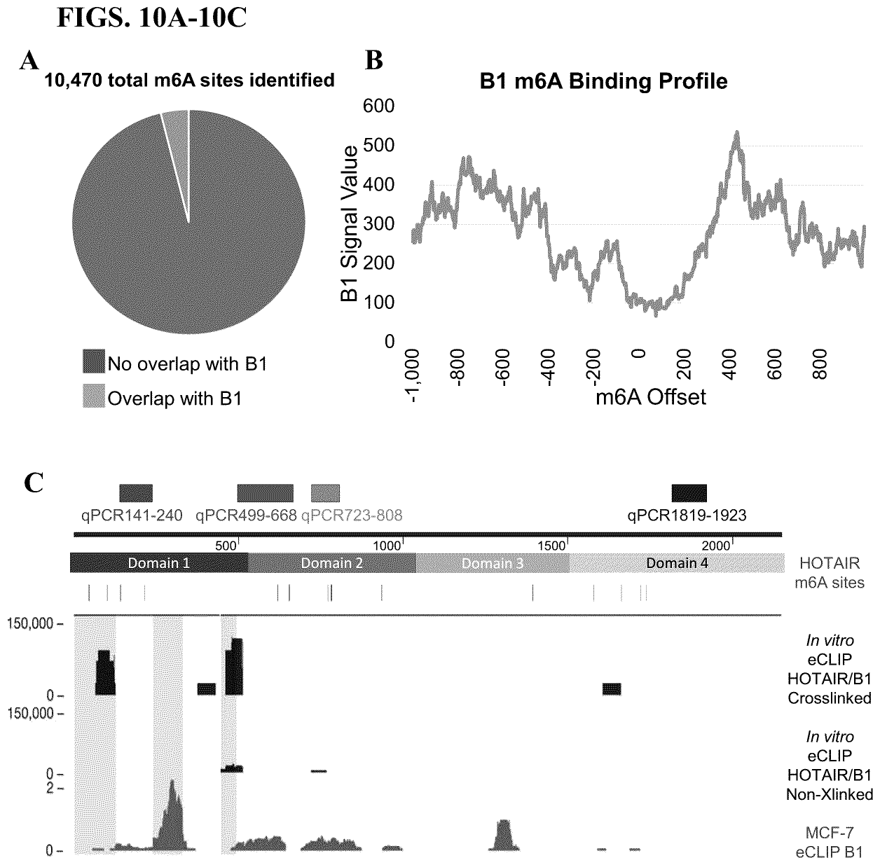

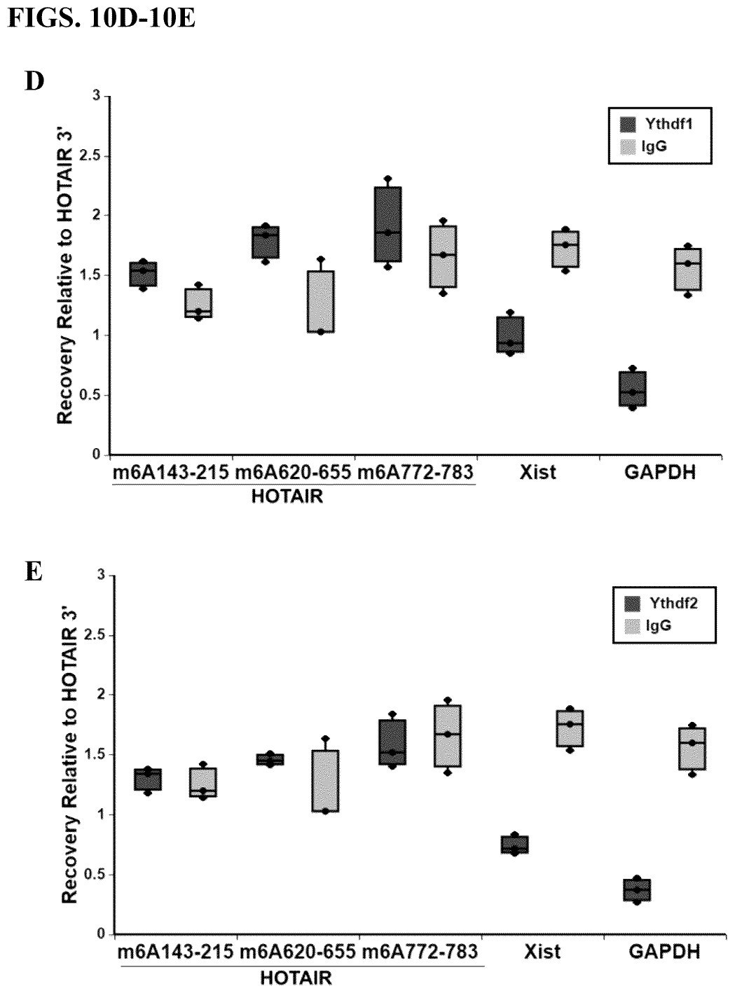

In another exemplary method, hnRNP A2/B1 was thought to be a reader of m6A, and the B1 isoform had a high affinity for binding HOTAIR. However, comparing previously generated eCLIP results for hnRNP B1 to the m6A eCLIP, both performed in MCF-7 cells, out of 10,470 m6A sites, only 417 (4%) were identified to contain an hnRNP B1 binding site within 1,000 nucleotides (FIG. 10A). Upon mapping hnRNP B1 signal intensity relative to the nearby m6A site, hnRNP B1 was depleted directly over m6A sites (FIG. 10B). These results suggest that hnRNP B1 was not a direct m6A reader, although m6A may indirectly promote its recruitment in some contexts. When comparing hnRNP B1 binding in HOTAIR with m6A sites, B1 binding peaks in MCF-7 cells occurred in m6A-free regions of HOTAIR. Conversely, data from in vitro eCLIP analysis of B1 binding to unmodified HOTAIR revealed additional B1 binding peaks in Domain 1 of HOTAIR, one of which occurred near several m6A sites (FIG. 10C). Altogether, these data suggested that m6A was not likely to directly recruit hnRNP B1 as a reader, although it could contribute to hnRNP B1 binding.

### Example 3

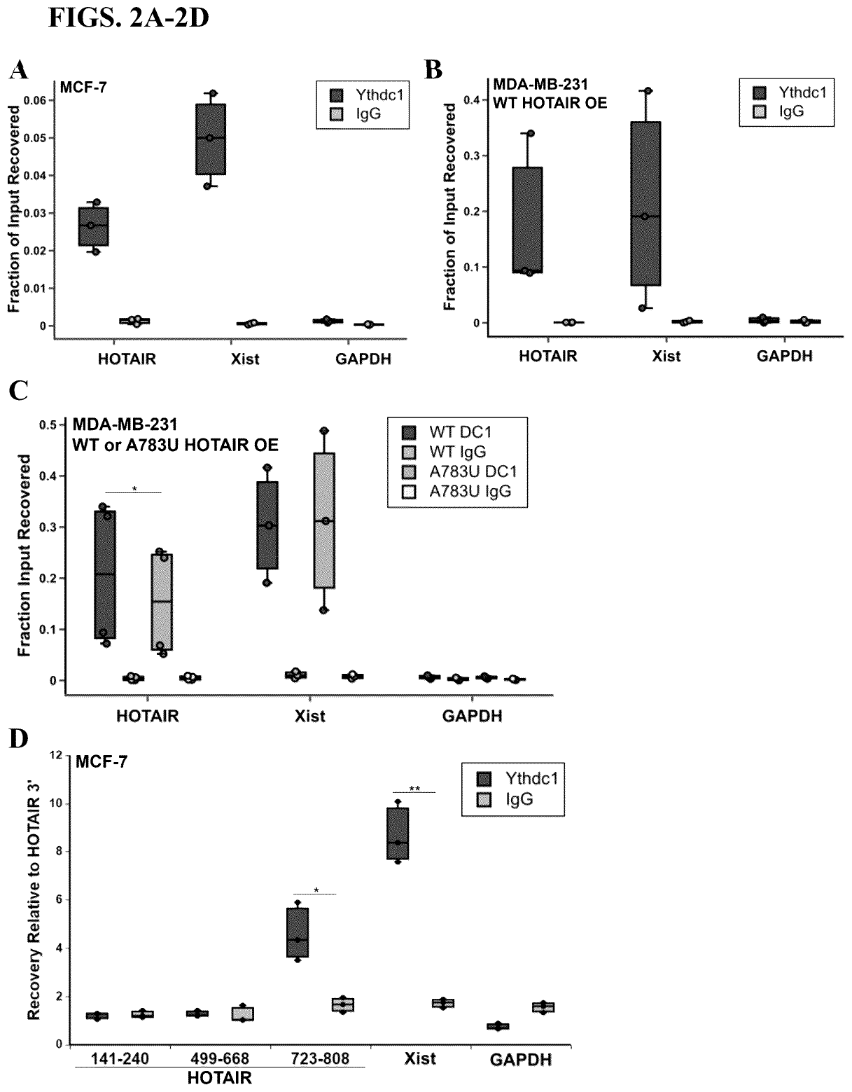

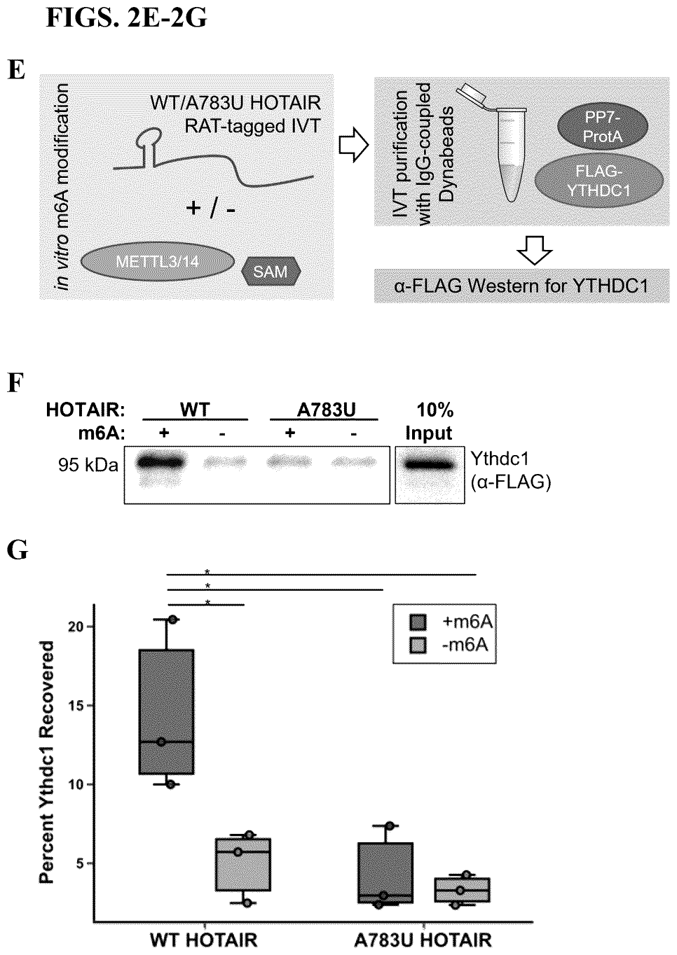

In another exemplary method to analyze m6A function on HOTAIR, RNA immunoprecipitation (RIP) of effector protein YTHDC1 using an antibody to YTHDC1 was performed and qRT-PCR was used to detect HOTAIR or controls. In both MDA-MB-231 cells overexpressing transgenic HOTAIR and MCF-7 cells expressing endogenous HOTAIR where m6A783 is the only m6A site detected, a significant portion of HOTAIR RNA was recovered when using antibodies specific against YTHDC1 (17.4%, p=0.04; 2.6%, p=0.003, respectively) (FIGS. 2A-2B). RIP experiments on MDA-MB-231 cells overexpressing WT or A783U HOTAIR demonstrated a significant reduction in HOTAIR RNA obtained from YTHDC1 RIP (0.73-fold change, p=0.042) (FIG. 2C). To further determine interaction of YTHDC1 at m6A783, YTHDC1 RIP experiments in MCF-7 cells, where A783 is the only methylated site detected, were conducted using qPCR oligos targeting different regions of HOTAIR. Here, it was found that there was a significant enrichment in recovery of the region containing m6A783 compared to other regions of HOTAIR (FIG. 2D), supporting m6A-dependent association at A783. To examine the association of other m6A readers, a similar RIP experiment was performed using antibodies to YTHDF1 or YTHDF2. While a proportion of HOTAIR was recovered with these antibodies, there was no enrichment for the region containing m6A783 (FIGS. 10D-10E). This supported a specific role for YTHDC1 as the m6A reader that targets m6A783 of HOTAIR.

To further confirm that nucleotide A783 in HOTAIR recruited YTHDC1 via m6A modification, a PP7-tagged in vitro transcribed RNA of domain 2 of WT or A783U mutant HOTAIR was in vitro m6A methylated with purified METTL3/14 and S-adenosylmethionine as a methyl donor (FIG. 2E). A783 has low structural propensity, making it a favorable METTL3/14 substrate. In this context, the only difference in methylation between WT and A783U HOTAIR should be at A783, as this is the only difference between the two transcripts. The in vitro HOTAIR transcripts were tethered to magnetic beads and incubated with FLAG-YTHDC1-containing protein lysates, then the relative recovery of FLAG-YTHDC1 was determined by anti-FLAG Western Blot (FIG. 2F). WT HOTAIR interaction with YTHDC1 was enhanced when the transcript was m6A-modified (˜3-fold increase, p=0.04), while A783U HOTAIR interaction with YTHDC1 was not significantly altered by the addition of m6A (˜1.3-fold change, p=0.6) (FIG. 2G). Altogether, these results suggested that m6A783 of HOTAIR mediates a specific interaction with YTHDC1.

### Example 4

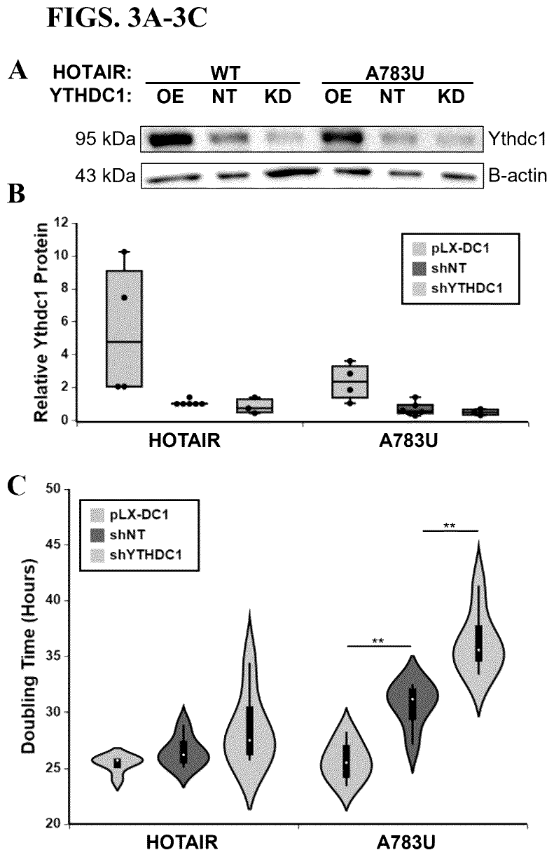

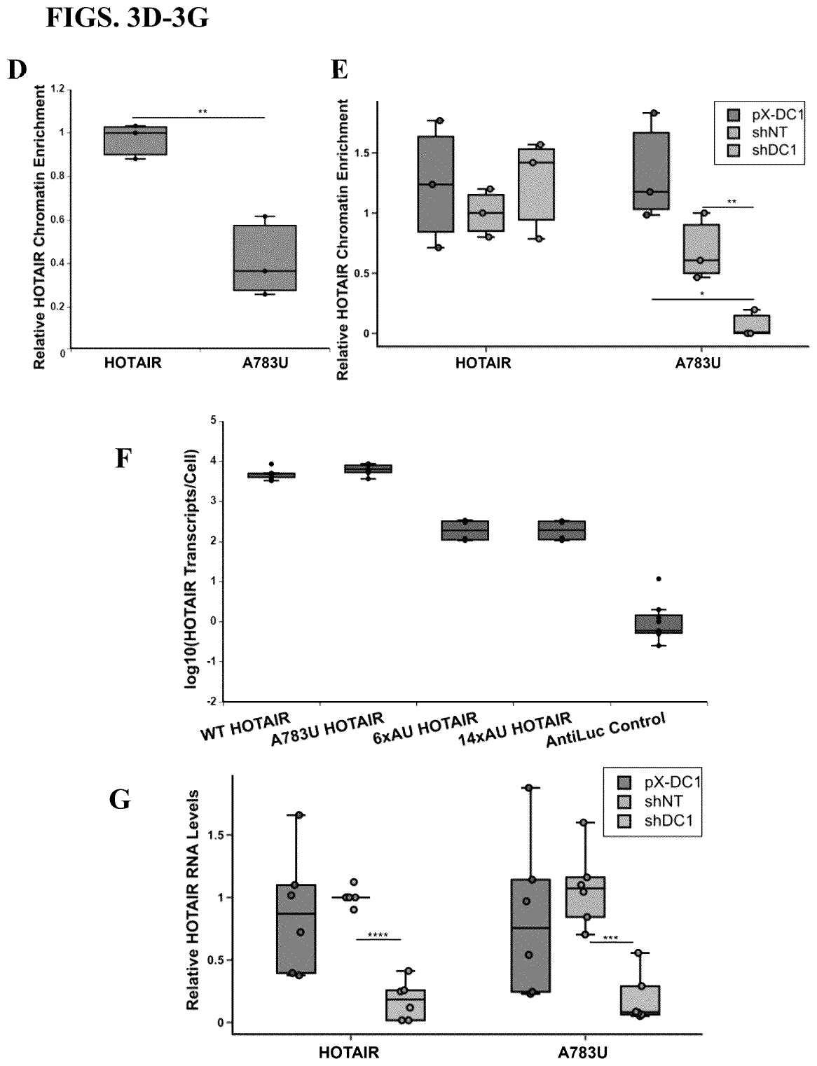

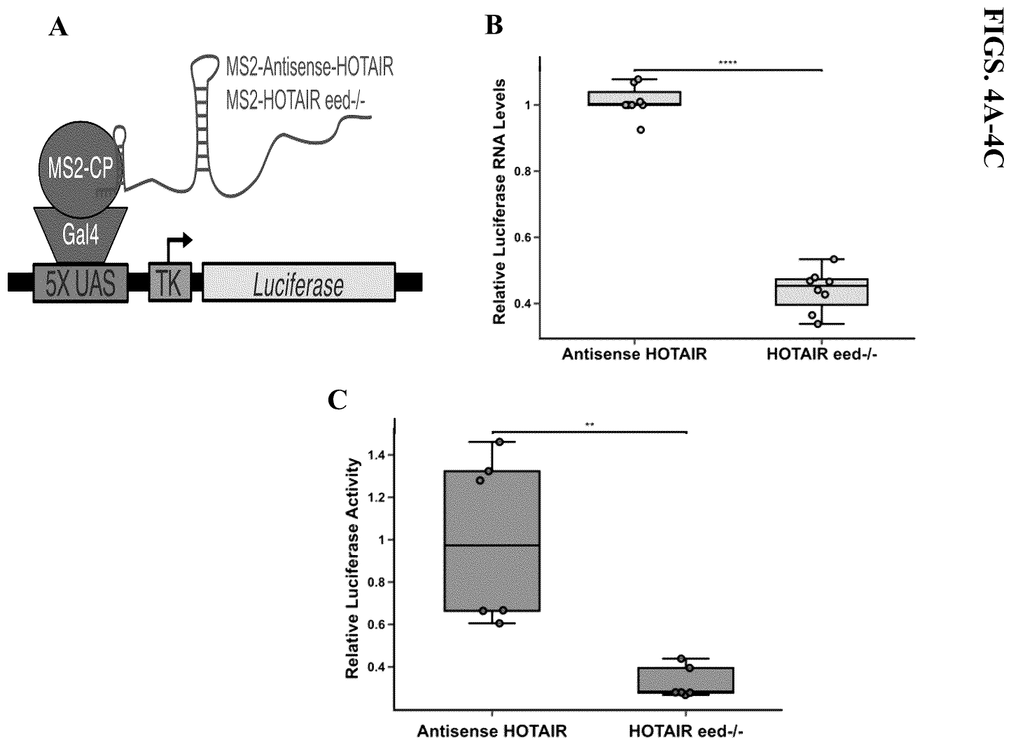

In another exemplary method to test the role of YTHDC1 in HOTAIR's ability to enhance breast cancer cell proliferation, stably overexpressed or knocked down YTHDC1 in the context of WT or A783U HOTAIR overexpression in MDA-MB-231 cells was achieved (FIG. 3A). YTHDC1 protein levels tended to be ˜2-fold higher in cells containing WT HOTAIR compared to A783U mutant HOTAIR (FIG. 3B). Although this difference was not significant (p=0.16), it suggested a potential positive relationship between WT HOTAIR RNA and YTHDC1 protein levels, indicating that high levels of methylated A783 may stabilize a fraction of YTHDC1. Next, the MDA-MB-231 cell lines were analyzed for proliferation as described above. Growth of MDA-MB-231 cells overexpressing WT HOTAIR was not significantly altered by YTHDC1 dosage (0.96 fold change, p=0.16 for pLX-DC1; 1.08 fold change, p=0.26 for shDC1, respectively), yet there was a trend towards decreased doubling time with increasing YTHDC1. In contrast, cells with A783U mutant HOTAIR had significant differences in doubling time with overexpression or knockdown of YTHDC1 (FIG. 3C). Overexpression of YTHDC1 led to significantly faster growth of MDA-MB-231 cells containing A783U mutant HOTAIR (0.84-fold change in doubling time, p=0.003), with proliferation rates comparable to cells expressing WT HOTAIR. Knockdown of YTHDC1 in cells containing HOTAIR^{A783U} was particularly potent in reducing the growth rate (˜1.2-fold increase in doubling time, p=0.008). These results suggest that without A783 methylation, the reduced occupancy of YTHDC1 specifically at A783 can be partially compensated by YTHDC1 overexpression and aggravated by knockdown. This could be mediated by the secondary m6A sites of HOTAIR, permitting some level of compensatory function.

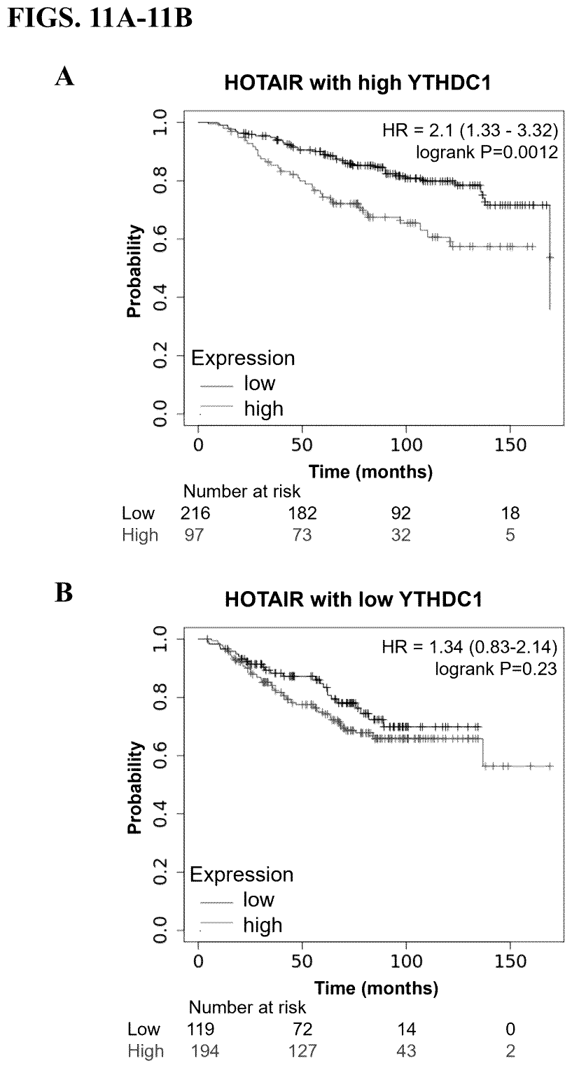

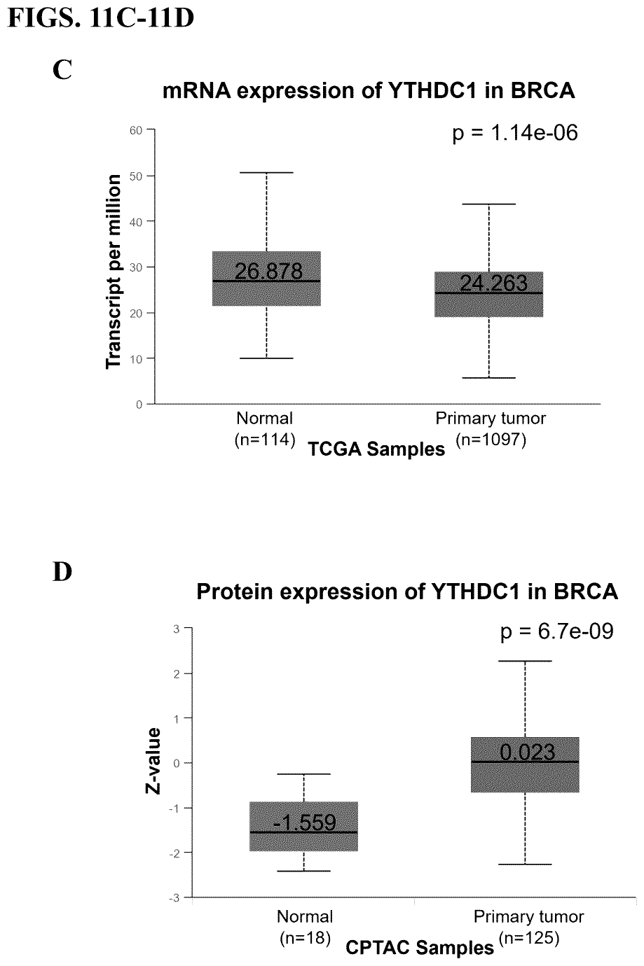

To explore how breast cancer outcomes are affected by HOTAIR and YTHDC1 levels, Kaplan-Meier Plotter was used to find that high HOTAIR levels were only significantly associated with decreased survival in the context of high YTHDC1 mRNA (FIGS. 11A-11B), suggestive of a role for YTHDC1 in enhancing HOTAIR's ability to mediate more aggressive cancer. Using UALCAN (a tool for analyzing cancer OMICS data) to determine gene expression in normal breast tissue versus breast tumor specimens, there appeared to be a lack of correlation in the relationship between YTHDC1 mRNA and protein levels in clinical samples: average YTHDC1 mRNA levels were decreased across a breast tumor panel, while average protein levels were increased (FIGS. 11C-11D). However, most samples with high YTHDC1 mRNA levels were likely to have higher protein levels. These data suggest that the cancer phenotypes dependent on HOTAIR association with YTHDC1 may have clinical implications. Discovery of methylated A783 in cells recently derived from a breast tumor (Table 3) support this as well.

### Example 5

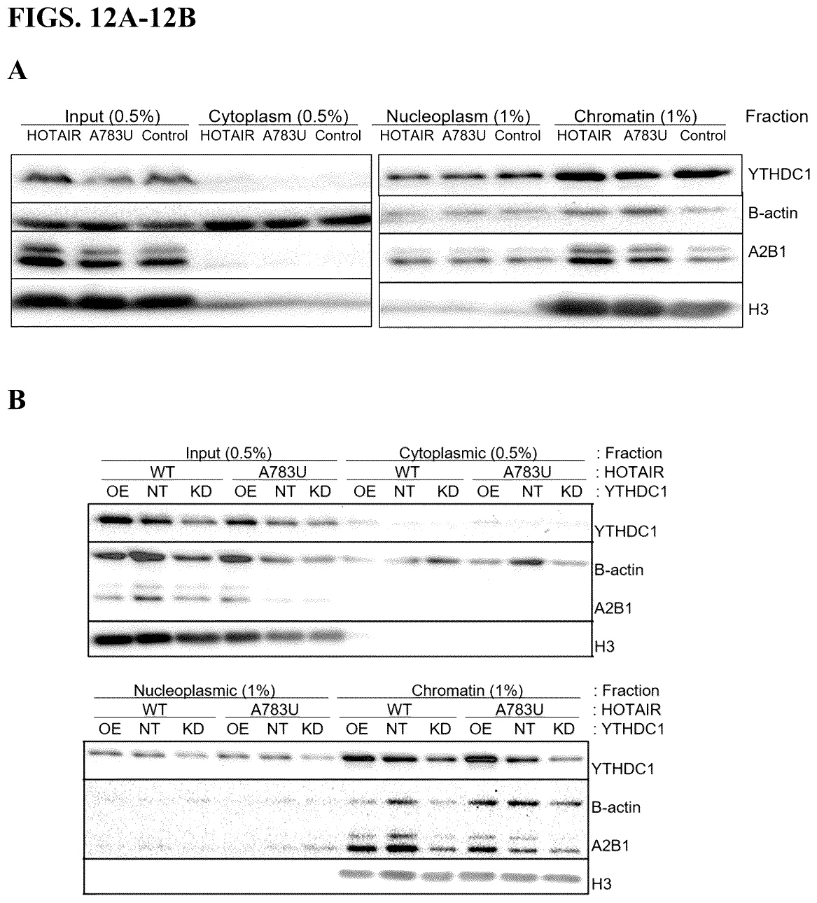

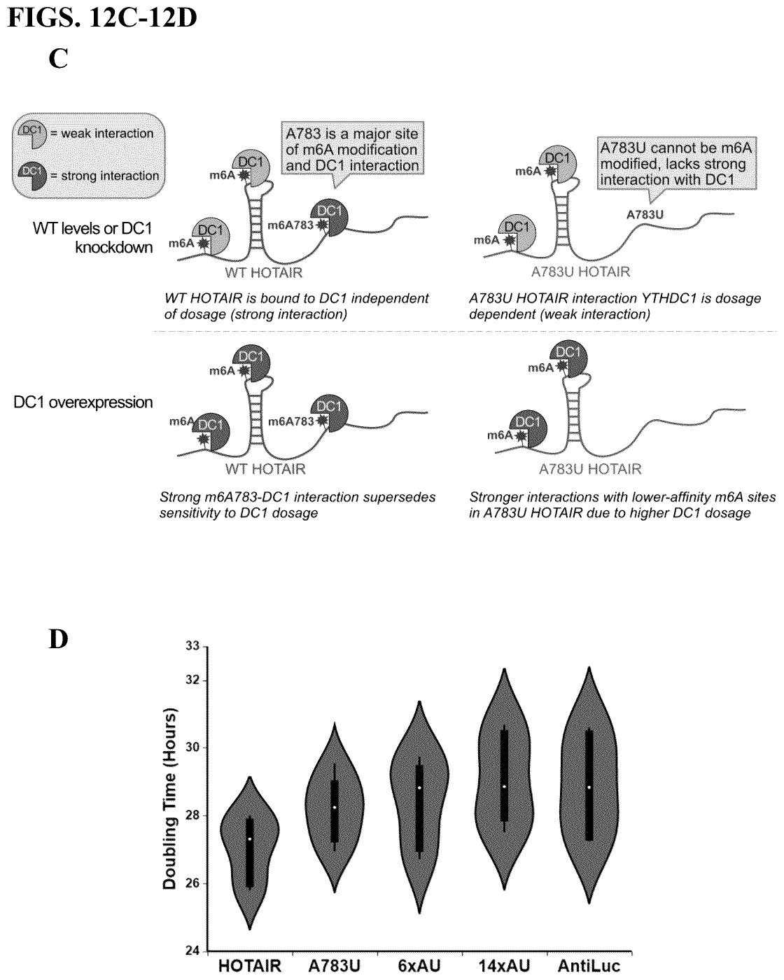

In another exemplary method, based on the differences observed between cell lines containing WT and A783U HOTAIR and the function of HOTAIR in chromatin-mediated gene repression, chromatin association of HOTAIR was profiled in these cells. Fractionation of MDA-MB-231 cells containing WT or A783U HOTAIR or an antisense-Luciferase control into cytoplasm, nucleoplasm, and chromatin fractions (FIG. 12A) was performed. RNA was isolated from each fraction and qRT-PCR for HOTAIR and GAPDH was performed. Cells overexpressing WT HOTAIR had significantly more chromatin-associated HOTAIR (˜4.3-fold) than cells expressing A783U HOTAIR (p<0.05) (FIG. 3D), though overall levels of HOTAIR are unchanged (FIG. 1E).

To examine the effect of YTHDC1 levels on HOTAIR chromatin association, a similar fractionation experiment in MDA-MB-231 cells expressing WT or A783U HOTAIR with overexpression or partial knockdown of YTHDC1 was performed (FIG. 12B). While YTHDC1 levels did not significantly alter WT HOTAIR chromatin association, overexpression of YTHDC1 increased HOTAIR^{A783U} chromatin association ˜1.9-fold to similar levels as WT HOTAIR (p=0.05), and knockdown resulted in a significant ˜10-fold decrease in chromatin association (p=0.01) (FIG. 3E). HOTAIR expression levels remained similar for DC1 overexpression lines compared to shNT control lines (0.8-fold change, p=0.3), but were significantly decreased by ˜5 to 10-fold in YTHDC1 knockdown lines for both WT and A783U mutant HOTAIR relative to shNT cell lines (p=3.24e-10) (FIG. 3F). Hence, while DC1 knockdown had no effect on relative WT HOTAIR chromatin association, total HOTAIR RNA levels were decreased, including chromatin associated HOTAIR, which may explain a partial effect in FIG. 3C. The differences observed between WT and A783U mutant HOTAIR were likely due to a high-affinity interaction of YTHDC1 with WT HOTAIR at m6A783 that enabled chromatin association and was not affected by partial knockdown or overexpression.

YTHDC1 levels had a 40% reduction upon knockdown, leaving a reduced, but substantial, population for interaction with methylated A783 of WT HOTAIR. For A783U mutant HOTAIR that did not interact with YTHDC1 at this position, increasing the concentration of YTHDC1 can drive interaction at other m6A sites within the mutated HOTAIR, each of which has lower affinity for YTHDC1 and/or lower frequency of m6A modification, but on average results in at least one site being occupied. These interactions occurred at a low level in cells with wild-type YTHDC1 levels and were most sensitive to knockdown of YTHDC1 (FIG. 12C). Therefore, the A783U mutant, which only retained these proposed lower affinity sites and could not properly compensate for loss of the critical A783 methylation, was particularly sensitive to YTHDC1 levels.

The fact that HOTAIR expression levels were significantly decreased in YTHDC1 knockdown lines for both WT and A783U mutant HOTAIR relative to shNT cell lines suggested that YTHDC1 regulated the expression or stability of HOTAIR, independently of A783. To investigate the role of other m6A sites within HOTAIR, HOTAIR overexpression constructs containing 6 or 14 adenosine-to-uracil mutations (6×AU and 14×AU, respectively) both of which included A783U were generated. While WT and A783U HOTAIR expression levels were similarly high, there was a ˜50-fold decrease in expression of 6×AU or 14×AU HOTAIR (FIG. 3G). This suggested that other m6A sites within HOTAIR mediated its high expression levels in breast cancer cells. Due to their decreased HOTAIR expression levels, longer doubling times were also observed for the multiple m6A HOTAIR mutants, similar to the single A783U mutant (FIG. 12D).

### Example 6

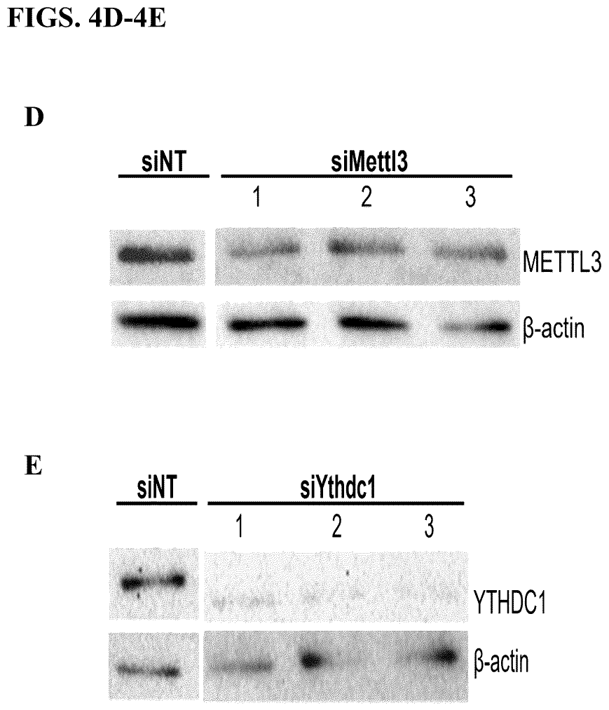

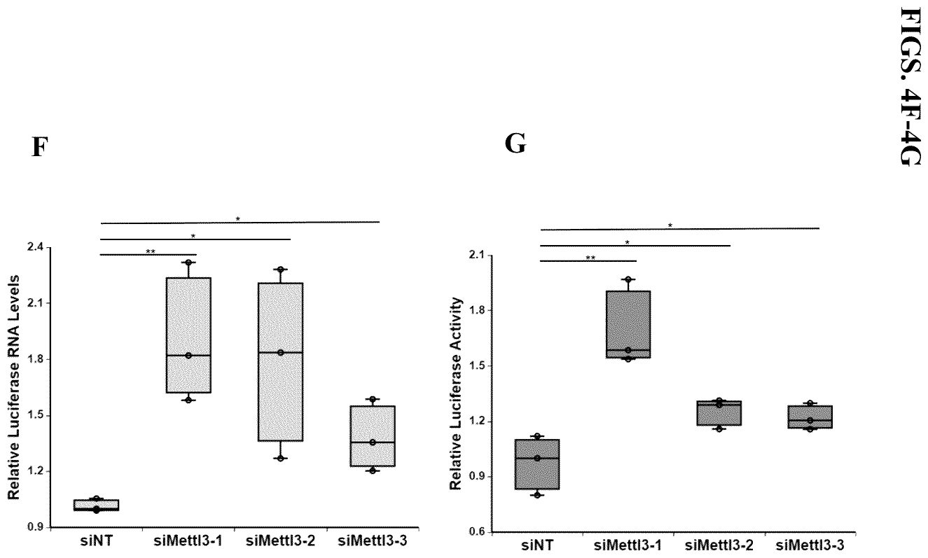

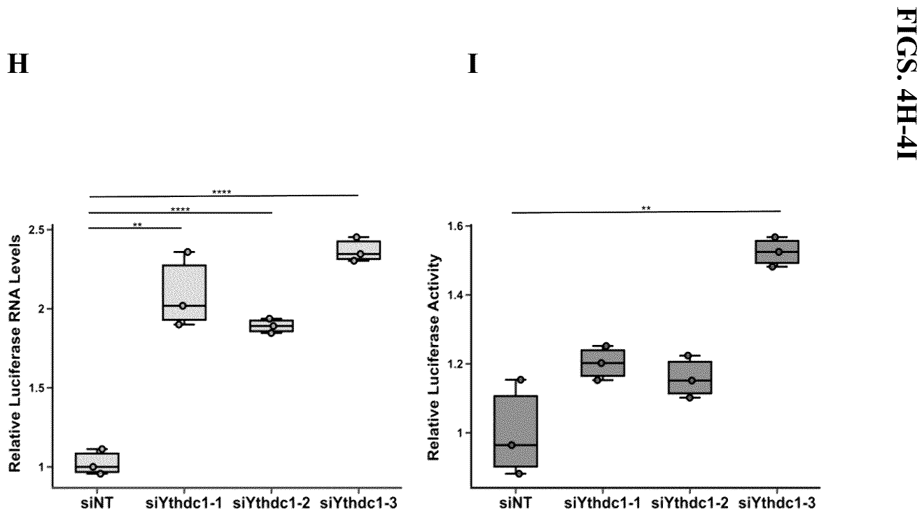

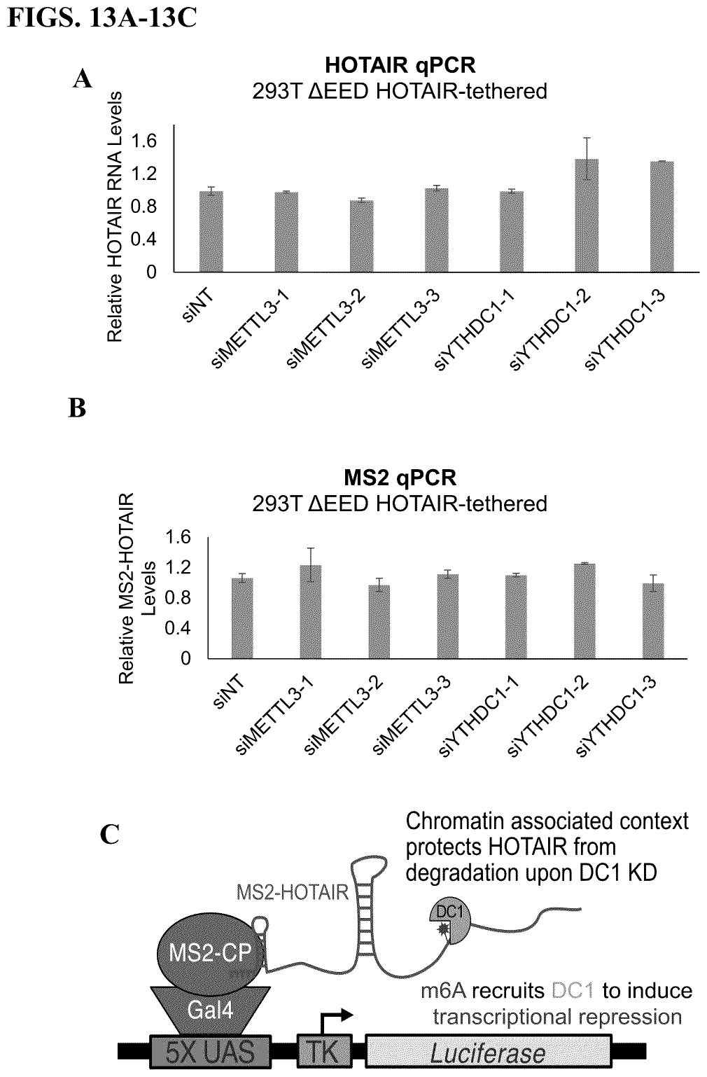

In another exemplary method, using previously generated reporter cell lines that contained HOTAIR artificially directly tethered to chromatin upstream of a luciferase reporter gene to repress expression, independent of PRC2 (FIG. 4A), it was confirmed that HOTAIR tethered upstream of the luciferase reporter reduced luciferase expression using both qRT-PCR (˜2.3-fold lower, p=8.0e-12) and luciferase assay (˜3.1-fold lower, p=0.002) (FIGS. 4B-4C). m6A eCLIP confirmed that HOTAIR was m6A modified in this context and detected 10 m6A sites within HOTAIR, including A783 (Table 1). To test the role of m6A and YTHDC1 in the repression mediated by HOTAIR, 3 different siRNAs were used to knock down METTL3 or YTHDC1 relative to a non-targeting control (˜1.5-fold decrease in METTL3 protein levels, p=0.04; ˜ 2-fold decrease in YTHDC1 protein levels, p=0.02) in the HOTAIR-tethered cells lacking the essential PRC2 subunit EED (FIGS. 4D and 4G). Knockdown of METTL3 or YTHDC1 resulted in significantly higher luciferase RNA levels in these cells (˜1.7-fold change for METTL3, p=0.0003; ˜ 2.1 fold change for YTHDC1, p=2.2e-05) (FIGS. 4E and 4H). Luciferase enzymatic activity also increased upon knockdown of either METTL3 or YTHDC1 (˜1.4 fold change for METTL3, p=0.01; 1.3 fold change for YTHDC1, p=0.03) (FIGS. 4F and 4I). Knockdown did not affect HOTAIR RNA levels in this context (FIGS. 13A and 13B), indicating that the effects observed on luciferase expression were likely due to disruption of the HOTAIR gene repression mechanism via depletion of METTL3 or YTHDC1 protein rather than loss of HOTAIR expression (FIG. 13C).

### Example 7

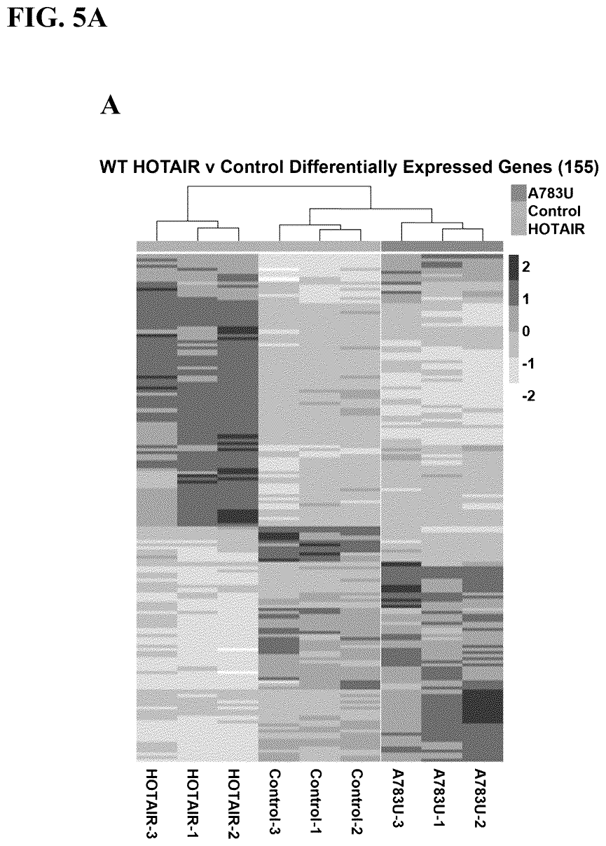

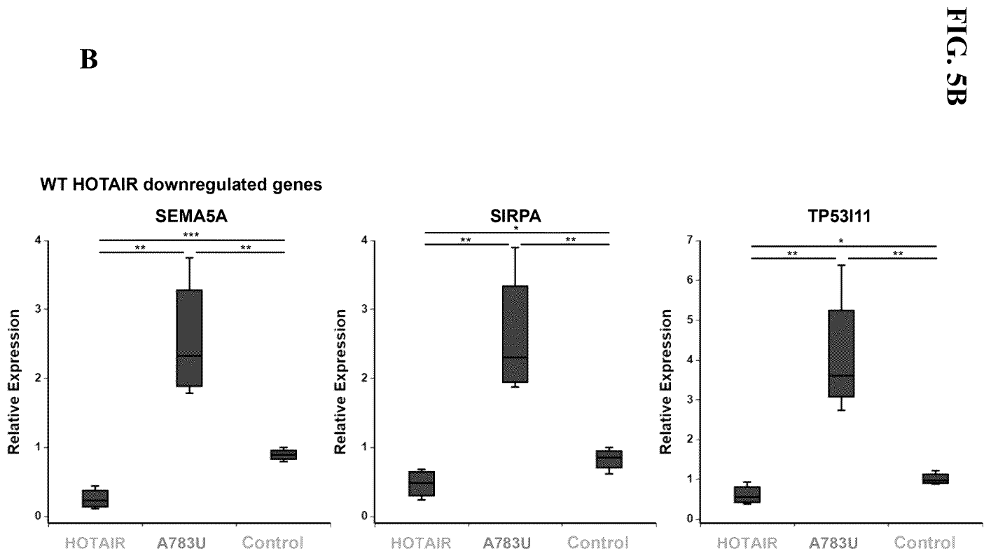

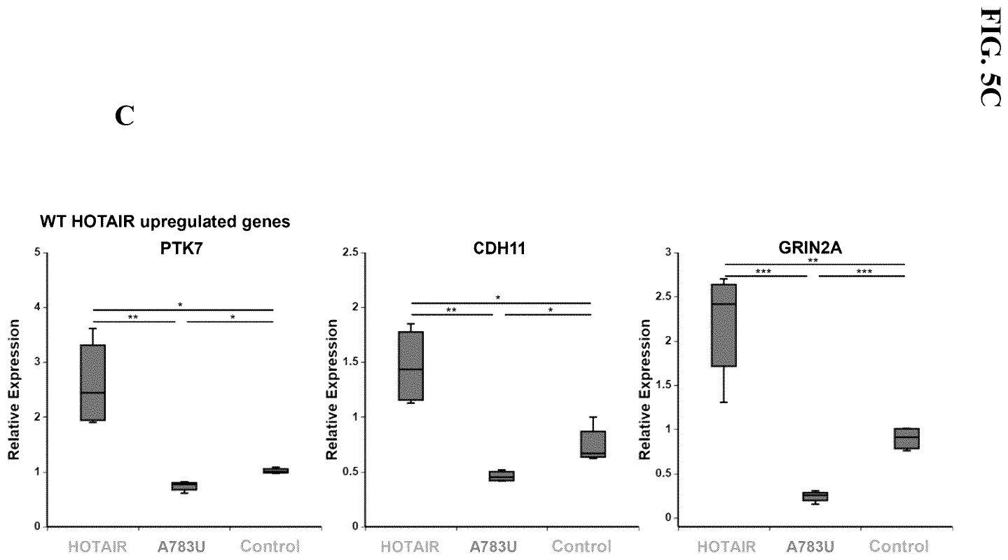

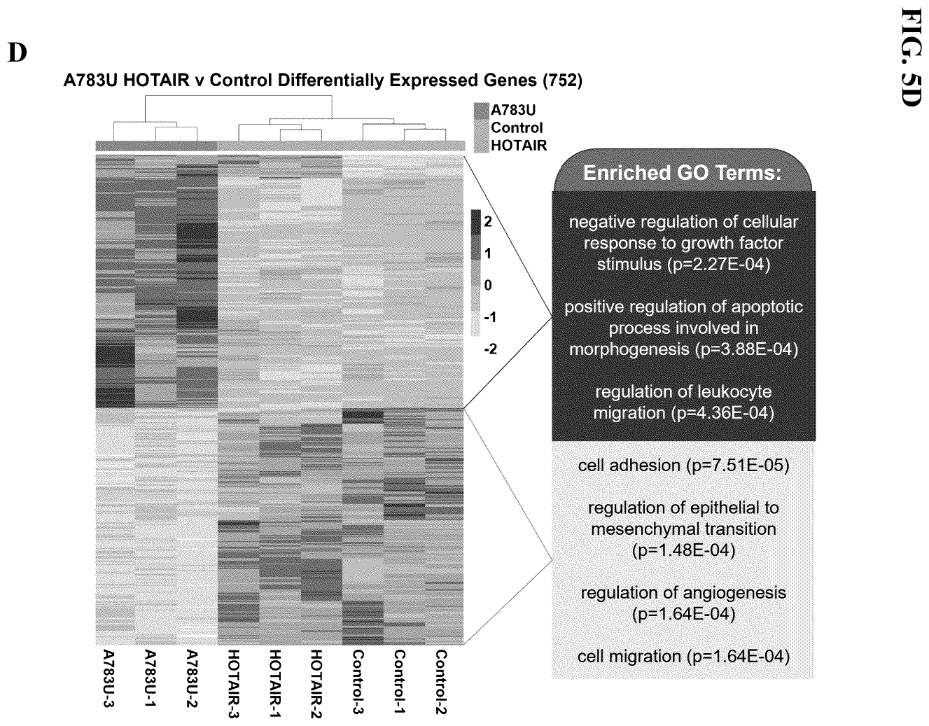

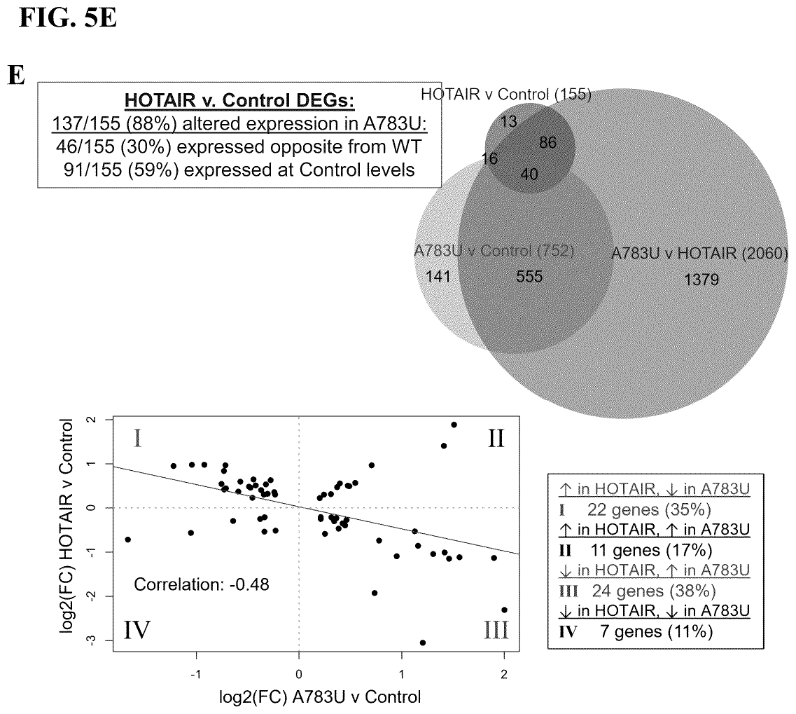

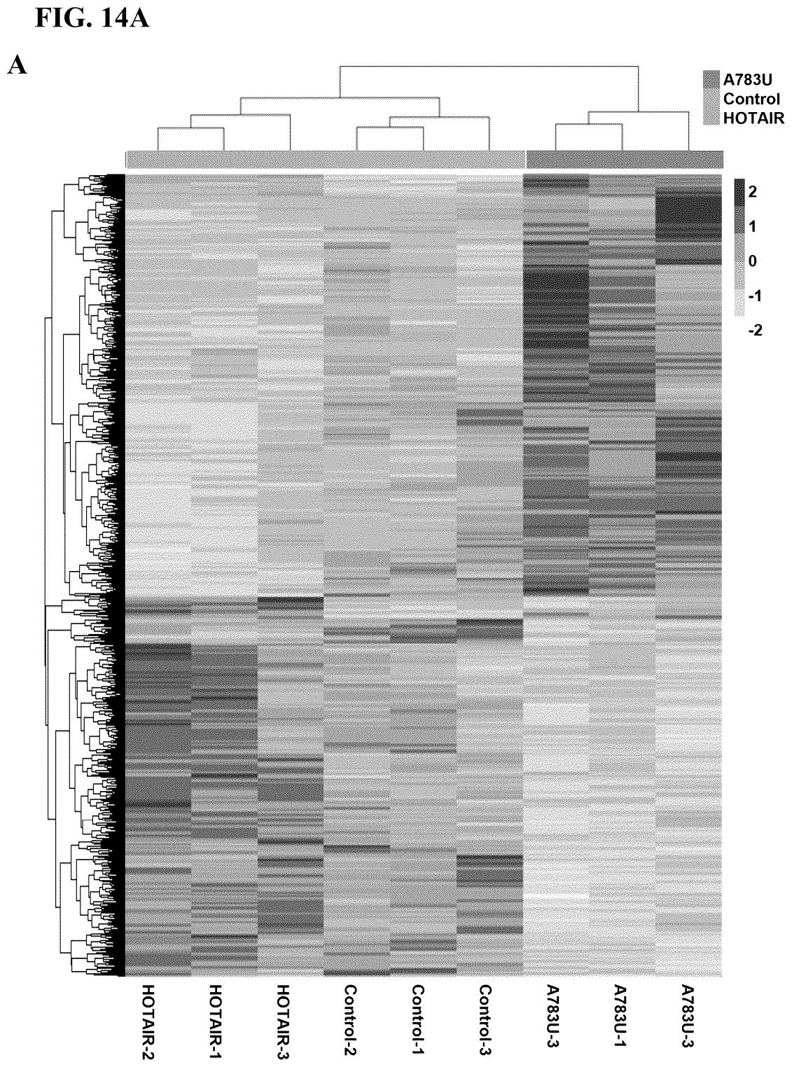

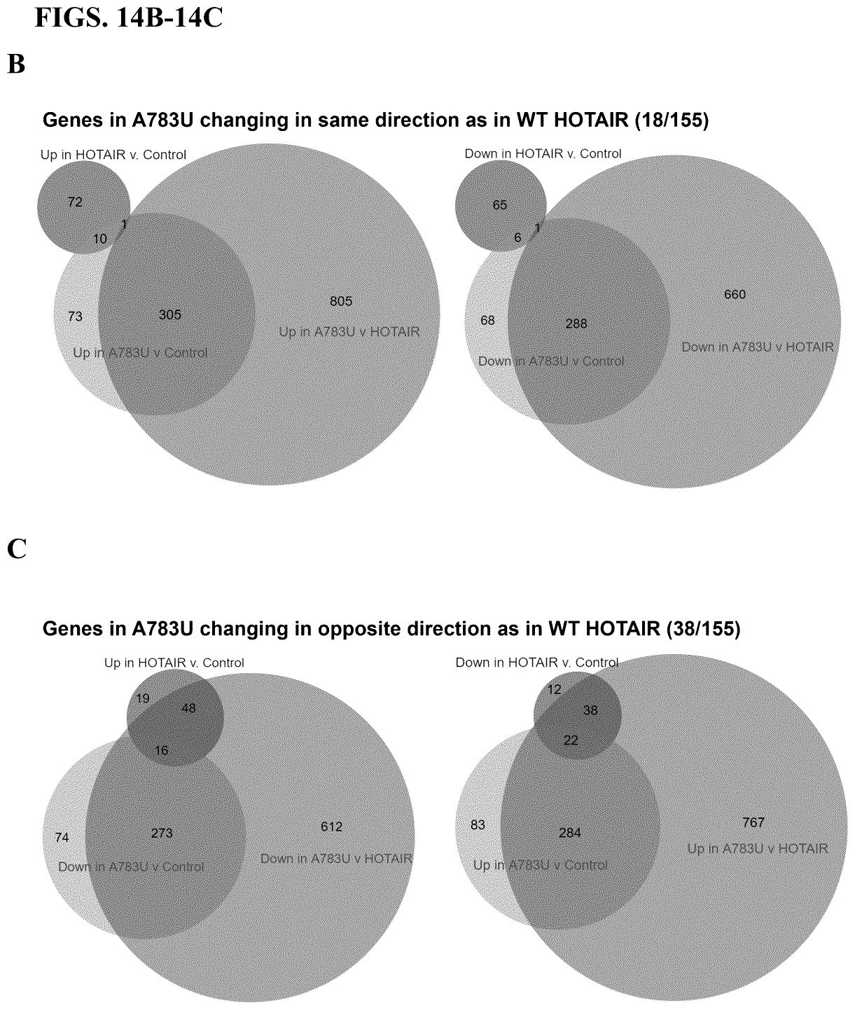

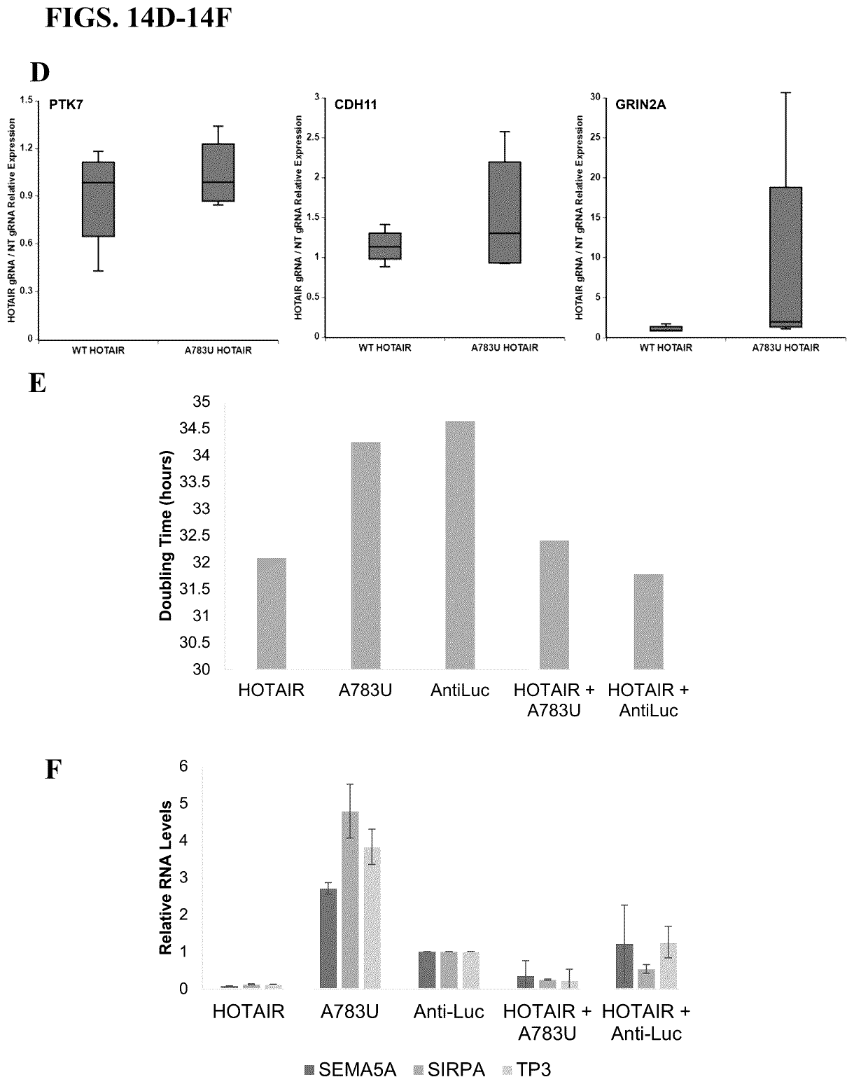

In another exemplary method to analyze HOTAIR-mediated gene expression changes in MDA-MB-231 cells, high throughput RNA sequencing was performed on cells overexpressing WT HOTAIR, A783U mutant HOTAIR, or the Anti-Luciferase control. For cells expressing WT HOTAIR, 156 genes were identified that were differentially expressed when compared with control cells expressing Anti-Luciferase (FIG. 5A). Upregulated genes in cells expressing WT HOTAIR included genes involved in positive regulation of angiogenesis (p=1.22E-05), regulation of cell population proliferation (p=0.0361), and cell differentiation (p=0.0157), while downregulated genes included genes involved in cell adhesion (p=0.0118), p53 (p=0.0112) and MAPK (p=0.0313) signaling, and tumor repressors such as HIC1 and DMNT3A. This set of genes had significantly different expression in cells overexpressing WT HOTAIR compared to either cells overexpressing Anti-Luciferase or the A783U mutant HOTAIR (FIGS. 5A-5C). Surprisingly, mutation of A783 did not only prevent most gene expression changes observed in wild-type HOTAIR, but it was also observed that expression of the A783U mutant induced changes in the opposite direction from the baseline control MDA-MB-231 cell line. This pattern was confirmed by qRT-PCR: genes downregulated with WT HOTAIR were significantly increased with A783U HOTAIR, compared to the parental MDA-MB-231 control (FIG. 5B) and genes upregulated by WT HOTAIR were significantly decreased (FIG. 5C). To further analyze differences in cells expressing A783U mutant HOTAIR, a pairwise comparison was conducted with control MDA-MB-231 cells that identified 758 differentially expressed genes (FIG. 5D). Upregulated gene categories in A783U HOTAIR-expressing cells include negative regulation of response to growth factor stimulus (p=2.27E-04), positive regulation of apoptosis (p=3.88E-04), and regulation of migration (p=4.36E-04), while downregulated gene categories include regulation of the epithelial to mesenchymal transition (p=1.48E-04), angiogenesis (p=1.64E-04), cell adhesion (p=7.51E-05), and cell migration (p=1.64E-04). This different pattern of gene expression may underlie the lower cell invasion observed in the A783U context compared to control MDA-MB-231 cells (FIG. 1H). The most differentially expressed genes (2060) were observed in the pairwise comparison between cells expressing WT HOTAIR and those expressing the A783U mutant HOTAIR (FIGS. 14A-14C).

These exemplary results revealed that at least one methylation site at A783 or equivalent position thereof in HOTAIR, A783U mutant HOTAIR, induced additional and often opposite gene expression changes compared to expression of WT HOTAIR in breast cancer cells, suggesting a potential antimorph property of this single nucleotide mutation. The opposite gene expression pattern was evident in the heat map of all differentially expressed genes (FIG. 2C), as well as the observation that the majority (137/155, ˜88%) of WT HOTAIR-regulated genes have altered gene expression with ˜30% significantly in the opposite expression in MDA-MB-231 cells expressing A783U HOTAIR (FIG. 5E).

In another exemplary method, to analyze YTHDC1 interaction with HOTAIR, a catalytically inactive RNA-targeting Cas protein, dCasRX, which has previously been used to recruit effectors to specific RNA molecules via a guide RNA, was used. A plasmid containing the dCasRX-YTHDC1 fusion protein, in combination with a plasmid containing either a HOTAIR guide RNA (targeting a 22-nucleotide sequence 7 nucleotides downstream from A783 in HOTAIR, see FIG. 6A) or a non-targeting gRNA were transfected into MDA-MB-231 cells stably expressing WT or A783U HOTAIR. Expression of dCasRX-YTHDC1 was confirmed by Western blot (FIG. 6B). While chromatin association of WT HOTAIR remained consistently high, chromatin association levels of A783U HOTAIR were only restored to near WT HOTAIR levels upon transfection with plasmids containing the dCasRX-YTHDC1 fusion protein and the HOTAIR gRNA (p=0.25 compared to WT HOTAIR). In contrast, chromatin association of A783U HOTAIR remained low upon transfection of dCasRX-YTHDC1 with a non-targeting guide RNA (˜3.7 fold lower than WT HOTAIR, p=0.0066) (FIG. 6C). HOTAIR RNA levels remained consistent in all samples (FIG. 6D). These results confirmed YTHDC1-mediated chromatin localization of HOTAIR and demonstrated that the chromatin association defect of the A783U mutation could be restored simply by restoring binding of YTHDC1 at that specific methylation location, A783. Doubling time (FIG. 6E) and invasion (FIG. 6F) were partially restored upon tethering of YTHDC1 to A783U mutant HOTAIR, further supporting YTHDC1's role in mediating these effects.

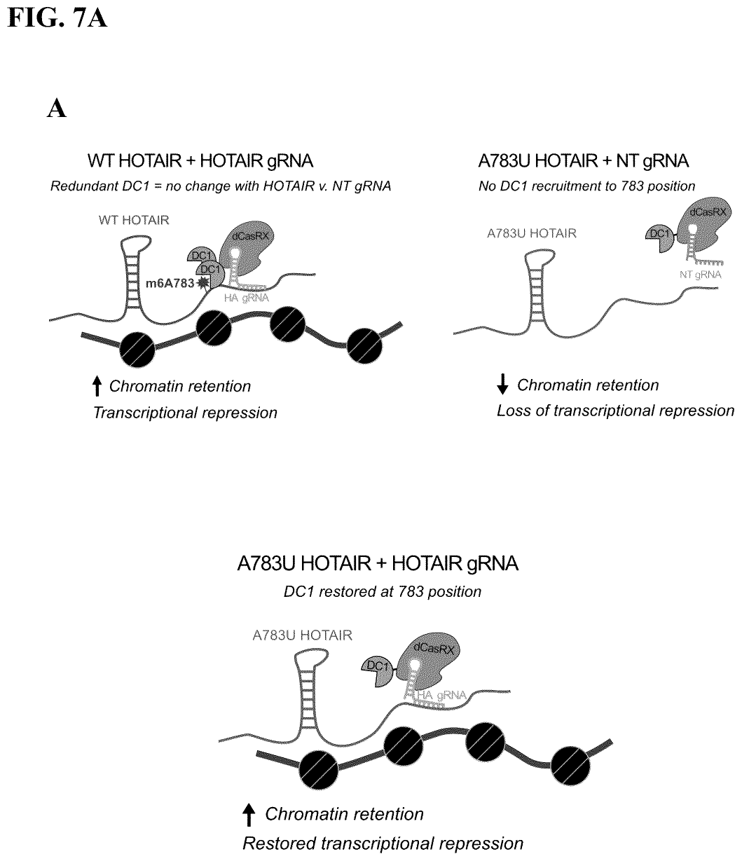

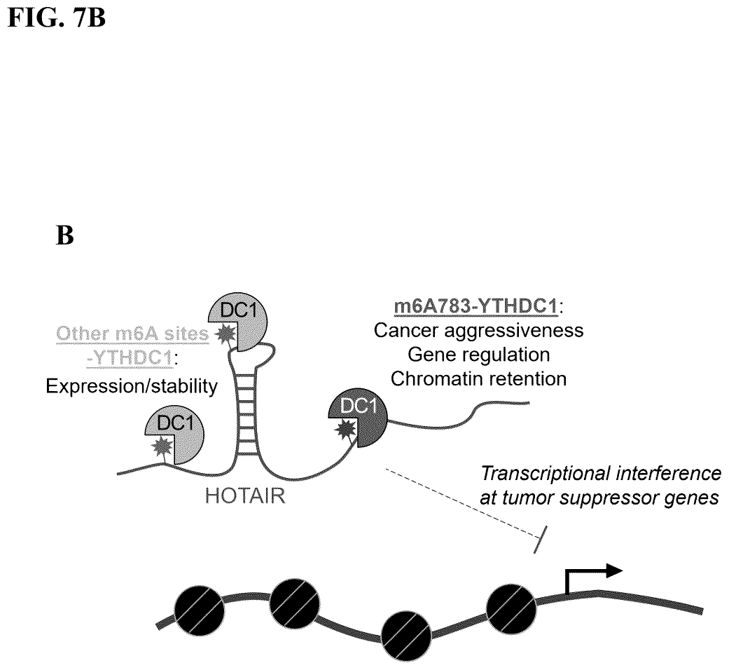

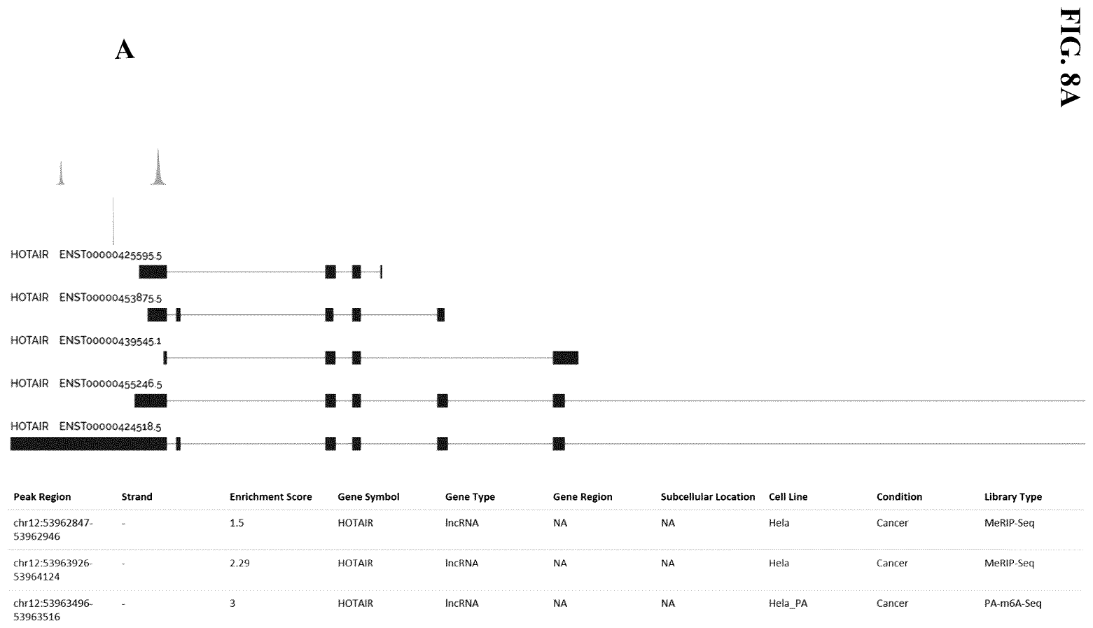

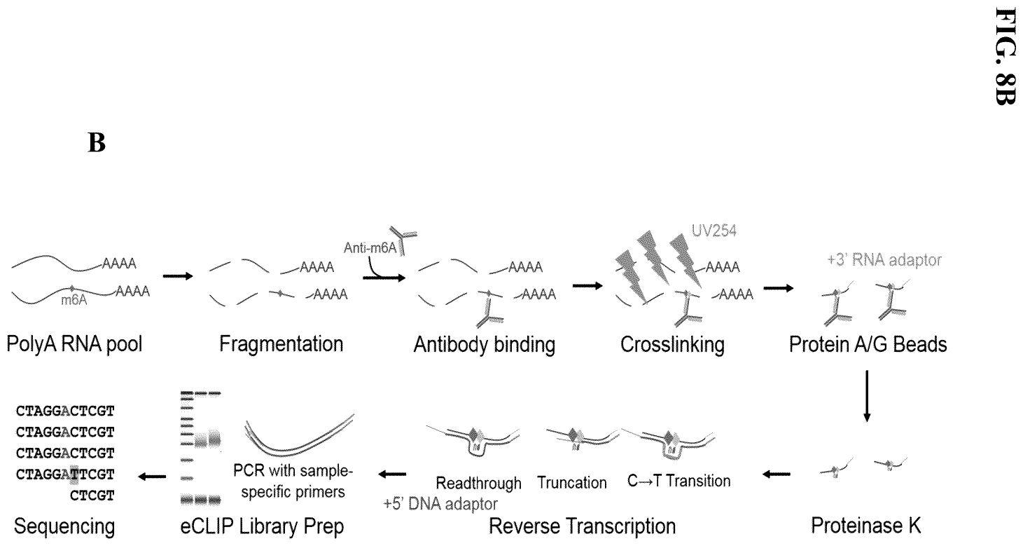

In another exemplary method to examine changes in HOTAIR-mediated gene expression upon tethering of YTHDC1 to A783U HOTAIR, qRT-PCR on the same transfections described above was performed. Tethering YTHDC1 to WT HOTAIR did not result in changes in gene expression of the HOTAIR-repressed genes. Without being bound by theory, the lack of changes in gene expression may be because WT HOTAIR was already bound by YTHDC1 at m6A783, so tethering additional YTHDC1 was redundant. However, tethering YTHDC1 to A783U HOTAIR resulted in reduced expression of these genes (FIG. 6G), suggesting a partial restoration of WT HOTAIR regulation. Genes upregulated with WT HOTAIR did not reveal significant changes in expression upon YTHDC1 tethering to A783U HOTAIR (FIG. 14D). Together, these exemplary results supported a mechanism where YTHDC1 interaction specifically at m6A783 led to repression of HOTAIR genomic targets, and that this direct action caused further gene expression changes, cumulatively promoting HOTAIR- and m6A-dependent cancer cell phenotypes (FIGS. 7A and 7B).

### Example 8

In another exemplary method to determine HOTAIR m6A site mutant function, cells expressing WT HOTAIR were transduced with a second transgene of A783U, or control RNA. No significant pattern of dominant negative behavior was observed with WT and A783U in the same cell, for doubling time or gene expression (FIGS. 14E and 14F).

### Materials and Methods

Exemplary methods as used in the illustrative examples herein are described as follows. These exemplary methods can also be used with any of the embodiments disclosed herein and are not limited to the exact method or systems disclosed as one of skill in the art readily recognizes.

### Cell Culture

In exemplary cell culture methods, MCF-7 cells were maintained in RPMI media and MDA-MB-231 and 293T cells were maintained in DMEM media. Media contained 10% FBS and Pen-Strep and cells were grown under standard tissue culture conditions. Cells were split using Trypsin (Fisher Scientific) according to manufacturer's instructions.

MDA-MB-231 cells overexpressing WT HOTAIR, A783U mutant HOTAIR, or Anti-Luciferase were generated using retroviral transduction. Stable knockdown of METTL3, METTL14, WTAP, and YTHDC1 and overexpression of YTHDC1 was performed by lentivirus infection of MCF-7 cells or MDA-MB-231 cells overexpressing HOTAIR or A783U mutant HOTAIR via Fugene HD R.8 with pLKO.1-blasticidin shRNA constructs or a pLX304 overexpression construct as noted in Table 3. Cells were selected with 5 μg/mL blasticidin. The nontargeting shRNA pLKO.1-blast-SCRAMBLE was obtained from Addgene. Two shRNAs for each target were obtained and stable lentiviral transductions with the targeted shRNAs and the scramble control were performed. Cell lines with the most efficient knockdown as determined by Western blot were selected for downstream experiments. Table 4 provides a list of exemplary shRNAs and ORFs used certain studies.

TABLE 4

| Gene/Knockdown or | |
|---|---|
| Overexpression | shRNA/ORF Used |
| METTL3 Knockdown | TRCN0000034715, TRCN0000034717 |
| MEETTL14 Knockdown | TRCN0000015933, TRCN0000015937 |
| WTAP Knockdown | TRCN0000231422, TRCN0000231424 |
| YTHDC1 Knockdown | TRCN0000243987, TRCN0000243989 |
| YTHDC1 Overexpression | ORF clone ccsdBroad304_04559 |

### Plasmid Construction.

The pBABE-puro retroviral vector was used for overexpression of lncRNAs. The spliced HOTAIR transcript (e.g. NR_003716.3 Homo sapiens HOX transcript antisense RNA (HOTAIR), transcript variant 2, long non-coding RNA (2,364 bp) other variants are known for example, Variant 1 (NR_047517.1) has 2,370 bp. Variant 3 (NR_047518.1) has 2,337 bp) was synthesized and cloned into the pBABE-puro retroviral vector by GenScript. The transcript disclosed herein for WT HOTAIR is represented by SEQ ID NO: 1 is 2148 bp and about 99% homologous to variant 2. An antisense transcript of the firefly luciferase gene (AntiLuc) was amplified from the pTRE3G-Luciferase plasmid (Clonetech), then cloned into the pBABE-puro retroviral vector.

In one exemplary method, to create the A783U mutant HOTAIR overexpression plasmid, staggered QuikChange oligos AG66/AG67 were used to generate the A783U mutation in pTRE3G-HOTAIR using the QuikChange Site Directed Mutagenesis Kit (Agilent 200519) to generate pTRE3G-A783U_HOTAIR. A 1.6 Kb fragment of A783U mutant HOTAIR was amplified with primers AG68/AG69 from pTRE3G-A783U_HOTAIR for cloning into pBABE-Puro-HOTAIR cut with XcmI and BamHI by Gibson Assembly.

All constructs were confirmed by sequencing. pBABE-Puro-6×AU_HOTAIR and pBabe-Puro-14×AU_HOTAIR were synthesized and cloned by GenScript.

Plasmids for the knockdown of METTL3, METTL14, WTAP, and YTHDC1 were generated by cloning the shRNA (RNAi Consortium shRNA Library) from pLKO.1-puro into the pLKO.1-blast backbone.

To generate the plasmid for tethering YTHDC1 to HOTAIR via dCasRX, a pCDNA-FLAG plasmid was created by inserting a 5×FLAG sequence (synthesized as a gBlock by IDT DNA) into the HindIII/XbaI site of pCDNA3. YTHIDC1 was then amplified from pLX304-YTHDC1 with oligonucleotides provided in Table 2 and cloned into the KpnI/NotI site of pCDNA-FLAG to generate pCDNA-FLAG-YTHDC1 (pAJ367). The FLAG-YTHDC1 sequence was amplified then cloned downstream of dCasRX at NheI in the pXR002 plasmid (pXR002: EF1a-dCasRx-2A-EGFP) using oligonucleotides provided in Table 5.

TABLE 5

| Experiment | Forward Oligo | Reverse Oligo |
|---|---|---|
| A783U | AG66 | AG67 |
| HOTAIR | CGCCCAGAGAtCGCTGGAAAAA | CCAGCGaTCTCTGGGCGTTCATG |
| QuikChange | CCTGAGCGG: SEQ. ID NO: 25 | TGGCGAGC SEQ. ID NO: 26 |
| A783U | AG68 | AG69 |
| HOTAIR | CCTAAACCAGCAATTACACCCA | CTGTGCTGGCGAATTCCTACGT |
| pBABE- | AGCTCGTTGGGGCCTAAG | ACCACCACACTGGGATCCGAAA |
| Puro | SEQ. ID NO: 27 | ATGCATCCAGATATTAAT |
| | | SEQ. ID NO: 28 |
| Cloning | AG64 | AG65 |
| YTHDC1 | ggtaccGCGGCTGACAGTCGGG | gcggccgccCTAATCTTCTATA |
| into | AGGAG | TCGACCTCTC |
| pCDNA3- | SEQ. ID NO: 29 | SEQ. ID NO: 30 |
| FLAG | | |
| Nhel cloning | AL01 | AL02 |
| YTHDC1 | gtagctgctagcGACTACAAAGA | gatgctgctagcTCTTCTATAT |
| into pXR002 | CGATGACGATAAAGGGG | CGACCTCTCTCCCCTCG |
| | SEQ. ID NO: 31 | SEQ. ID NO: 32 |
| HOTAIR | AL05 | AL10 |
| gRNA | AAACCCCCGGCACCCGCTCAGG | AAAAAAAACCTGAGCGGGTGCC |
| cloning into | TTTT SEQ. ID NO: 33 | GGGG SEQ. ID NO: 34 |
| pXR003 | | |
| Non- | AL07 | AL 11 |
| targeting | AAACCAGAAGCGTACCATACTC | AAAATCGTGAGTATGGTACGCT |
| gRNA | ACGA SEQ. ID NO: 35 | TCTG SEQ. ID NO: 36 |
| cloning into | | |
| pXR003 | | |

Expression of the dCasRX-YTHDC1 fusion protein was confirmed by transfection of the plasmid followed by Western Blot with anti-FLAG M2 mouse monoclonal antibody and anti-YTHDC1. Plasmids containing guide RNAs were generated using the pXR003 backbone plasmid (pXR003: CasRx gRNA cloning backbone was cut with BbsI, using oligonucleotides disclosed herein). All plasmids were confirmed by sequencing. Further plasmid information, oligonucleotides, and additional exemplary methods used in the following examples are provided below.

m6A Enhanced Crosslinking Immunoprecipitation polyA Isolation and RNA Fragmentation.

For each experiment, approximately 100 μg of total RNA was isolated from cells with TRIzol according to manufacturer's instructions. 10 μg PolyA RNA was isolated using Magnosphere Ultrapure mRNA Purification Kit (Takara) according to manufacturer's instructions. PolyA RNA was ethanol precipitated with 2.5 M Ammonium Acetate and 70% ethanol in a solution containing 50 μg/ml GlycoBlue Co-precipitant (AM9515, Invitrogen). RNA was resuspended in 10 μl and fragmented with 10× Fragmentation Buffer (AM8740, Invitrogen) at 75° C. for 8 minutes and immediately quenched with 10× Stop Reagent (AM8740, Invitrogen) and placed on ice to generate fragments 30-150 nucleotides in length. Anti-m6A-RNA crosslinking and bead conjugation

Crosslinked RNA-Antibody was generated. Fragmented RNA was resuspended in 500 μl Binding/Low Salt Buffer (50 mM Tris-HCl pH 7.4, 150 mM Sodium Chloride, 0.5% NP-40) containing 2 μl RNase Inhibitor (M0314, NEB) and 10 μl m6A antibody (ab151230, Abcam), and incubated for 2 hours at room temperature with rotation. RNA-Antibody sample was transferred to one well of a 12-well dish and placed in a shallow dish of ice. Sample was crosslinked twice at 150 mJ/cm² using a Stratagene Stratalinker UV Crosslinker 1800 and transferred to a new tube. 50 μl Protein A/G Magnetic Beads (88803, Pierce) were washed twice with Binding/Low Salt Buffer, resuspended in 100 μl Binding/Low Salt Buffer, and added to crosslinked RNA-Antibody sample. Beads were incubated at 4° C. overnight with rotation.

eCLIP Library Preparation

RNA was isolated and sequencing libraries were prepared using a modified enhanced CLIP protocol. Beads were washed twice with High Salt Wash Buffer (50 mM Tris-HCl pH 7.4, 1 M Sodium Chloride, 1 mM EDTA, 1% NP-40, 0.5% Sodium Deoxycholate, 0.1% Sodium Dodecyl Sulfate), once with Wash Buffer (20 mM Tris-HCl pH 7.4, 10 mM Magnesium Chloride, 0.2% Tween-20), once with Wash Buffer and 1× Fast AP Buffer (10 mM Tris pH 7.5, 5 mM Magnesium Chloride, 100 mM Potassium Chloride, 0.02% Triton X-100) combined in equal volumes, and once with 1× Fast AP Buffer. Beads were resuspended in Fast AP Master Mix (1× Fast AP Buffer containing 80U RNase Inhibitor (M0314, NEB), 2U TURBO DNase (AM2238, Invitrogen), and 8U Fast AP Enzyme (EF0654, Thermo Scientific)) was added. Samples were incubated at 37° C. for 15 minutes shaking at 1200 rpm. PNK Master Mix (1× PNK Buffer (70 mM Tris-HCl pH 6.5, 10 mM Magnesium Chloride), 1 mM Dithiothreitol, 200U RNase Inhibitor, 2U TURBO DNase, 70U T4 PNK (EK0031, Thermo Scientific)) was added to the samples and they incubated at 37° C. for 20 minutes shaking at 1200 rpm.

Beads were washed once with Wash Buffer, twice with Wash Buffer and High Salt Wash Buffer mixed in equal volumes, once with Wash Buffer, once with Wash Buffer and 1× Ligase Buffer (50 mM Tris pH 7.5, 10 mM Magnesium Chloride) mixed in equal volumes, and twice with 1× Ligase Buffer. Beads were resuspended in Ligase Master Mix (1× Ligase Buffer, 1 mM ATP, 3.2% DMSO, 18% PEG 8000, 16U RNase Inhibitor, 75U T4 RNA Ligase I (M0437, NEB)), two barcoded adaptors were added (X1a and X1b, see Table 6), and samples were incubated at room temperature for 75 minutes with flicking every 10 minutes. Beads were washed once with Wash Buffer, once with equal volumes of Wash Buffer and High Salt Wash Buffer, once with High Salt Wash Buffer, once with equal volumes of High Salt Wash Buffer and Wash Buffer, and once with Wash Buffer. Beads were resuspended in Wash Buffer containing 1× NuPAGE LDS Sample Buffer (NP0007, Invitrogen) and 0.1M DTT, and incubated at 70° C. for 10 minutes shaking at 1200 rpm.

Samples were cooled to room temperature and supernatant was ran on Novex NuPAGE 4-12% Bis-Tris Gel (NP0321, Invitrogen). Samples were transferred to nitrocellulose membrane, and membranes were cut and sliced into small pieces between 20 kDa and 175 kDa to isolate RNA-antibody complexes. Membrane slices were incubated in 20% Proteinase K (03508838103, Roche) in PK Buffer (100 mM Tris-HCl pH 7.4, 50 mM NaCl, 10 mM EDTA) at 37° C. for 20 minutes shaking at 1200 rpm. PK Buffer containing 7M urea was added to samples and samples were incubated at 37° C. for 20 minutes shaking at 1200 rpm. Phenol:Chloroform:Isoamyl Alcohol (25:24:1) (P2069, Sigma-Aldrich) was added to samples and samples were incubated at 37° C. for 5 minutes shaking at 1100 rpm. Samples were centrifuged 3 minutes at 16,000×g and aqueous layer was transferred to a new tube.

RNA was isolated using RNA Clean & Concentrator-5 Kit (R1016, Zymo) according to manufacturer's instructions. Reverse transcription was performed using AR17 primer (Table S5) and SuperScript IV Reverse Transcriptase (18090010, Invitrogen). cDNA was treated with ExoSAP-IT Reagent (78201, Applied Biosystems) at 37° C. for 15 minutes, followed by incubation with 20 mM EDTA and 0.1M Sodium Hydroxide at 70° C. for 12 minutes. Reaction was quenched with 0.1M Hydrochloric Acid. cDNA was isolated using Dynabeads MyONE Silane (37002D, ThermoFisher Scientific) according to manufacturer's instructions. 20% DMSO and rand3Tr3 adaptor (Table 6) was added to samples, and samples were incubated at 75° for 2 minutes. Samples were placed on ice and Ligation Master Mix (1×NEB Ligase Buffer, 1 mM ATP, 25% PEG 8000, 15U T4 RNA Ligase I (NEB)) was added to samples. Samples were mixed at 1200 rpm for 30 seconds prior to incubation at room temperature overnight.

TABLE 6

| m6A eCLIP | |
|---|---|
| oligonucleotides | Sequence |
| X1a (RNA) | /5Phos/rArUrArUrArGrG |
| | rNrNrNrNrNrArGrArUrCr |
| | GrGrArArGrArGrCrGrUr |
| | CrGrUrGrUrArG/3 SpC3/ |
| | SEQ ID NO: 37 |
| X1b (RNA) | /5Phos/rArArUrArGrCrA |
| | rNrNrNrNrNrArGrArUrCr |
| | GrGrArArGrArGrCrGrUrC |
| | rGrUrGrUrArG/3SpC3/ |
| | SEQ ID NO: 38 |
| Rand3Tr3 (RNA) | /5phos/rArGrArUrCrGrG |
| | rArArGrArGrCrGrUrCrGr |
| | UrG/3SpC3/ |
| | SEQ ID NO: 39 |
| RiL19 (RNA) | /5phos/rArGrArUrCrGrG |
| | rArArGrArGrCrGrUrCrGr |
| | UrG/3SpC3/ |
| | SEQ ID NO: 40 |
| AR17 (DNA) | /5Phos/NNNNNNNNNNAGAT |
| | CGGAAGAGCACACGTCTG/ |
| | 3SpC3/ |
| | SEQ ID NO: 41 |

cDNA was isolated using Dynabeads MyONE Silane according to manufacturer's instructions and eluted with 10 mM Tris-HCl pH 7.5. A 1:10 dilution of cDNA was used to quantify the cDNA library by qPCR using a set of Illumina's HT Seq primers, and Ct values were used to determine number of cycles for PCR amplification of cDNA. The undiluted cDNA library was amplified by combining 12.5 μL of the sample with 25 μL Q5 Hot Start PCR Master Mix and 2.5 μL (20 μM) of the same indexed primers used previously. Amplification for the full undiluted sample used 3 cycles less than the cycle selected from the diluted sample. The PCR reaction was isolated using HighPrep PCR Clean-up System (AC-60050, MAGBIO) according to manufacturer's instructions.

The final sequencing library was gel purified by diluting the sample with 1× Orange G DNA loading buffer and running on a 3% quick dissolve agarose gel containing SYBR Safe Dye (1:10,000). Following gel electrophoresis, a long wave UV lamp was used to extract DNA fragments from the gel ranging from 175 to 300 base pairs. The DNA was isolated using QiaQuick MinElute Gel Extraction Kit (28604, Qiagen). The purified sequencing library was analyzed via TapeStation using DNA ScreenTape (either D1000 or HS D1000) according to the manufacturer's instructions to assess for appropriate size and concentration (the final library should be between 175 and 300 base pairs with an ideal concentration of at least 10 nM).

### Sequencing and Analysis.

Samples were sequenced on an Illumina MiSeq or NovaSEQ6000 with 2×150 base pair paired-end reads to generate 40 million raw reads for each sample. Computational analysis methods are described. Briefly, a custom Snakemake workflow was generated based on the original eCLIP analysis strategies to map reads to the human genome. To identify m6A sites, a custom analysis pipeline was used to identify variations from the reference genome at single-nucleotide resolution across the entire genome. An internally developed Java package was used to identify C-to-T mutations occurring 1) within the m6A consensus motif ‘RAC’: ‘R’ is any purine, A or G; A being the methylated adenosine; and C where the mutation occurs; and 2) within a frequency range of greater than or equal to 2.5% and less than or equal to 50% of the total reads at a given position (with a minimum of 3 C-to-T mutations at a single site). The resulting m6A sites were then compared to those identified in the corresponding input sample and any sites occurring in both were removed from the final list of m6A sites (this eliminated any mutations that are not directly induced from the anti-m6A antibody crosslinking). Full transcriptome data associated with the methods manuscript is at GEO accession number GSE147440.

m6A RNA Immunoprecipitation (meRIP)

Total RNA was isolated with TRIzol (15596018, Invitrogen) according to the manufacturer's instructions. RNA was diluted to 1 μg/μl and fragmented with 1× Fragmentation Buffer (AM8740, Invitrogen) at 75° C. for 5 minutes. 1× Stop Reagent (AM8740, Invitrogen) was added immediately following fragmentation and samples placed on ice. 500 ng of input sample was reserved in 10 μl nuclease free water for qRT-PCR normalization. Protein A/G Magnetic Beads (88803, Pierce) were washed twice with IP Buffer (20 mM Tris pH 7.5, 140 mM NaCl, 1% NP-40, 2 mM EDTA) and coupled with anti-m6A antibody (ab151230, Abcam) or an IgG control (NB810-56910, Novus) for 1 hour at room temperature. Beads were washed 3 times with IP Buffer. 10 μg fragmented RNA and 400U RNase inhibitor was added to 1 ml IP Buffer. Antibody-coupled beads were resuspended in 500 μl RNA mixture and incubated 2 hours to overnight at 4° C. on a rotor. Beads were washed 5 times with cold IP Buffer. Elution Buffer (1× IP Buffer containing 10 U/μl RNase inhibitor and 0.5 mg/ml N6-methyladenosine 5′-monophosphate (M2780, Sigma-Aldrich) was prepared fresh and kept on ice. Samples were eluted with 200 μl Elution Buffer for 2 hours at 4° C. on a rotor. Supernatant was removed and ethanol precipitated with 2.5M Ammonium Acetate, 70% Ethanol, and 50 μg/ml GlycoBlue Coprecipitant (Invitrogen AM9515). RNA was washed with 70% ethanol, dried for 10 minutes at room temperature, and resuspended in 10 μl nuclease free water. RNA was quantified by nanodrop and 200 ng RNA was reverse transcribed using High Capacity cDNA Reverse Transcription Kit (4368814, ThermoFisher Scientific) and quantified by qPCR (oligonucleotides listed in Table 7), and fraction recovered was calculated from Input and IP values.

TABLE 7

| | qPCR Forward | qPCR Reverse |
|---|---|---|
| Gene | Primer | Primer |
| HOTAIR | AGACCCTCAG | CCCTACTGCA |
| (141- | GTCCCTAATA | GGCTTCTAAA |
| 240) | TC | TC |
| | SEQ ID | SEQ ID |
| | NO: 42 | NO: 43 |
| HOTAIR | GGTAGAAAAA | ACATAAACCT |
| (499- | GCAACCACGA | CTGTCTGTGA |
| 668) | AGC | GTGC |
| | SEQ ID | C |
| | NO: 44 | SEQ ID |
| | | NO: 45 |
| HOTAIR | GGGAACGGGA | GGCACCCGCT |
| (723- | GTACAGAGAG | CAGGTTT |
| 808) | AAT | SEQ ID |
| | A | NO: 47 |
| | SEQ ID | |
| | NO: 46 | |
| HOTAIR | TGATGCATGT | TCAGGCATTG |
| (1819- | AGACACAGAA | GGAATGGTAA |
| 1923) | GG | T |
| | SEQ ID | SEQ ID |
| | NO: 48 | NO: 49 |
| GAPDH | CCGGGAAACT | AGGTGGAGGA |
| | GTGGCGTGAT | GTGGGTGTCG |
| | GG | CTGTT |
| | SEQ ID | SEQ ID |
| | NO: 50 | NO: 51 |
| XIST | AAACCCAACA | GCGGTCACAC |
| | CGAAAAGCAC | AGGAAAAGAT |
| | SEQ ID | SEQ ID |
| | NO: 52 | NO: 53 |
| EEF1A1 | CGGTCTCAGA | AAACCAAAGT |
| +m6A | ACTGTTTGTT | GGTCCACAAA |
| | TC | SEQ ID |
| | SEQ ID | NO: 55 |
| | NO: 54 | |
| EEF1A1 | GGATGGAAAG | TTGTCAGTTG |
| distal | TCACCCGTAA | GACGAGTTGG |
| | G | SEQ ID |
| | SEQ ID | NO: 57 |
| | NO: 56 | |
| PTK7 | ACACTTCGTT | CAGCAGGAAT |
| | GCCACATTGA | ACAGCCCAC |
| | T | SEQ ID |
| | SEQ ID | NO: 59 |
| | NO: 58 | |
| CDH11 | AGAGGTCCAA | GGTTGTCCTT |
| | TGTGGGAACG | CGAGGATACT |
| | SEQ ID | GT |
| | NO: 60 | SEQ ID |
| | | NO: 61 |
| GRIN2A | TGGCCTCACC | CAATGCCGTC |
| | GGGTATGATT | CCTCACTCTC |
| | SEQ ID | SEQ ID |
| | NO: 62 | NO: 63 |
| SEMA5A | GATCCTGCCA | AGATGACACA |
| | TTTACCGAAG | AAGTTTGGCT |
| | C | CA |
| | SEQ ID | SEQ ID |
| | NO: 64 | NO: 65 |
| SIRPA | ACATGGTCCA | ACGCTGGCGT |
| | CCTCAACCG | ACTCTGAGA |
| | SEQ ID | SEQ ID |
| | NO: 66 | NO: 67 |
| TP53I11 | GAAGACCCGC | TTTCATTGCC |
| | AAGATCCTCG | TAAGACCTGG |
| | SEQ ID | C |
| | NO: 68 | SEQ ID |
| | | NO: 69 |
| Luciferase | GCACTGATCA | GAGAATAGGG |
| (LucR2) | TGAACTCCTC | TTGGCACCAG |
| | TGGA | CAG |
| | TCTAC | CGCAC |
| | SEQ ID | SEQ ID |
| | NO: 70 | NO: 71 |

### RNA Immunoprecipitation of YTHDC1

Actively growing cells from 70-90% confluent 15-cm dishes were trypsinized and washed twice with ice-cold 1×PBS. Cell pellet was resuspended in 1% V/V Formaldehyde (28908, Pierce) in 1×PBS and incubated at room temperature for 10 minutes on a rotor. Crosslinking was quenched with 0.25 M glycine at room temperature for 5 minutes. Cells were washed 3 times with ice-cold 1×PBS and placed on ice. 20 μl Protein A/G beads were washed twice with RIPA Binding Buffer (50 mM Tris-HCl pH 7.4, 100 mM Sodium Chloride, 1% NP-40, 0.1% Sodium Dodecyl Sulfate, 0.5% Sodium Deoxycholate, 4 mM Dithiothreitol, 1× Protease Inhibitors), resuspended in 1 ml RIPA Binding Buffer, and split to two 0.5 ml aliquots. 2 μg YTHDC1 antibody (ab122340, Abcam) or an IgG Control (sc-2027, Santa Cruz Biotechnology) was added to beads and incubated for 2 hours at 4° C. on a rotor. Fixed cells were resuspended in 1 ml RIPA Binding Buffer and placed in the Bioruptor Pico (B01060010, Diagenode) for 10 cycles of 30 seconds on, 30 seconds off. Lysates were digested with TURBO DNase for 5 minutes at 37° C. with mixing at 1000 rpm and transferred to ice for 5 minutes. Lysates were clarified by centrifugation at 17,000 g at 4° C. for 10 minutes and supernatant was transferred to a new tube. 200U RNase Inhibitor was added to the 1 ml clarified lysate. A 5% aliquot was removed and processed downstream with IP samples. A 2% aliquot was removed and diluted with 1×SDS Sample Buffer (62.5 mM Tris-HCl pH 6.8, 2.5% SDS, 0.002% Bromophenol Blue, 5% β-mercaptoethanol, 10% glycerol) and protein input and recovery was monitored by Western Blot. Antibody-coupled beads were washed 3 times with RIPA Binding Buffer and resuspended in half of the remaining lysate. Samples were incubated overnight at 4° C. on a rotor. Beads were washed 5 times with RIPA Wash Buffer (50 mM Tris-HCl PH 7.4, 1 M Sodium Chloride, 1% NP-40, 0.1% Sodium Dodecyl Sulfate, 0.5% Sodium Deoxycholate, 1 M Urea, 1× Protease Inhibitors) and resuspended in 100 μl RNA Elution Buffer (50 mM Tris-HCl pH 7.4, 5 mM EDTA, 10 mM Dithiothreitol, 1% Sodium Dodecyl Sulfate). Input sample was diluted with 1×RNA Elution Buffer. Formaldehyde crosslinks in both input and IP samples were reversed by incubation at 70° C. for 30 minutes at 1000 rpm. Supernatant was transferred to a new tube and RNA was isolated using TRIzol-LS according to the manufacturer's instructions. Reverse transcription was performed on 100 ng RNA using SuperScript IV Reverse Transcriptase. qPCR was performed as described below.

RNA Isolation and qRT-PCR.

RNA was isolated with TRIzol (Life Technologies) with extraction in chloroform followed by purification with the RNeasy kit (Qiagen). Samples were DNase treated using TURBO DNase (Ambion). Reverse transcription was performed using the cDNA High Capacity Kit (Life Technologies). qPCR was performed using Sybr Green master mix (Takyon, AnaSpec Inc) using the primers listed in Table 6 on a C1000 Touch Thermocycler (BioRad). The HOTAIR qPCR primer set targeting region 499-668 was used to quantify HOTAIR except where otherwise noted. EEF1A1 primer sequences were obtained from the Magna MeRIP m6A kit (17-10499, Sigma-Aldrich). Sequences for Luciferase primers (LucR2) were obtained from a previous publication. Three qPCR replicates were performed for each sample, and these technical replicates were averaged prior to analysis of biological replicates. At least 3 biological replicates were performed for each qPCR experiment.

Cell Proliferation Assays. Three independent clones, here defined as a pool of selected cells stably expressing the pBabe plasmid, were analyzed for cell proliferation. 2,000 cells were plated in a 96-well dish in DMEM media containing 10% FBS and selective antibiotics (1 μg/ml puromycin (P8833, Sigma-Aldrich) and/or 5 μg/ml blasticidin (71002-676, VWR)), allowed to settle at room temperature for 20 minutes, then placed in an Incucyte S3 (Sartorius). Pictures were taken with a 10× magnification every 2 hours for 48 hours using a Standard scan. Confluency was determined using the Incucyte ZOOM software. Growth rate was calculated from % confluency using the Least Squares Fitting Method.

### Cell Invasion Assays

MDA-MB-231 cell lines were grown to 70-90% confluence and serum starved in OptiMEM for ˜20 hours prior to setting up the experiment. Cells were washed, trypsinized, and resuspended in 0.5% serum DMEM. 10% serum DMEM was added to the bottom chamber of Corning Matrigel Invasion Chambers (Corning 354481), and 200,000 cells were plated in the top chamber in 0.5% serum DMEM. Cells were incubated for 22 hours at 37° C. followed by 4% PFA fixation and 0.1% Crystal Violet staining. Matrigel inserts were allowed to dry overnight, followed by brightfield imaging with a 20× air objective. Four biological replicates were performed, with technical duplicates in each set. For each Matrigel insert, four fields of view were captured, and cells were counted in Fiji (eight data points per condition, per biological replicate). The violin plot includes all of the data points, while statistical analysis was performed on the average number of cells/field for each biological replicate.

### Purification of METTL3/14

Methyltransferase like-3 (METTL3) and METTL14 complex transfers a methyl group from S-adenosyl-L-methionine to N6 amino group of adenosine. Suspension-adapted HEK293 cells (Freestyle 293-F cells, R790-07, Life Technologies) were grown as recommended in Freestyle 293 Expression Medium (12338026, Life Technologies) shaking at 37° C. in 5% CO₂. Cells were grown to a concentration of 3×10⁶ cells/ml and diluted to 1×10⁶ cells/ml in 50 ml 293F Freestyle Media 24 hours prior to transfection. Before transfection, cells were spun down and resuspended in 50 ml fresh 293F Freestyle Media at a concentration of 2.5×10⁶ cells/ml. Expression plasmid (pcDNA3.1-FLAG-METTL3, pcDNA3.1-FLAG-METTL14) were added to the flask at a concentration of 1.5 μg, and flask was shaken in the incubator for 5 minutes. 9 μg/ml PEI was added to the flask and cells were returned to incubator. After 24 hours of growth, an additional 50 ml fresh 293F Freestyle Media was added and culture was supplemented with 2.2 mM VPA. Cells were harvested as two 50 ml pellets 72 hours after addition of VPA.

Cell pellets were resuspended in 1× Lysis Buffer (50 mM Tris pH 7.4, 150 mM Sodium Chloride, 1 mM EDTA, 1% TritonX-100, 1× Protease inhibitors) to obtain a concentration of 10⁷ cells/ml and incubated for 20 minutes at 4° C. with rotation. Cell lysate was clarified by centrifugation at 4° C., 12,000×g for 15 minutes. Supernatant was transferred to a new tube and kept on ice. Anti-FLAG M2 affinity resin was equilibrated with 1× Lysis Buffer by washing 3 times. Equilibrated resin was resuspended in 1× Lysis Buffer and added to the tube containing the clarified lysate. Sample was incubated for 2 hours at 4° C. with rotation. Resin was pelleted by centrifugation at 4° C., 500×g. Supernatant was removed, and resin was washed 3 times with 1× Wash Buffer (50 mM Tris pH 7.4, 150 mM Sodium Chloride, 10% Glycerol, 1 mM Dithiothreitol) for 5 minutes each at 4° C. with rotation. Sample was equilibrated to room temperature and resin was resuspended in 1× Wash Buffer containing 0.2 mg/ml 3×FLAG Peptide. Samples were incubated at room temperature for 10 minutes shaking at 1000 rpm, centrifuged for 2 minutes at 1000×g, and supernatant was reserved (elution 1). Elution was repeated twice to obtain two additional elution samples (elution 2 and 3). Samples were analyzed by Coomassie to determine protein concentration and purity. Samples were aliquoted and stored at −80° C. and thawed on ice prior to use in in vitro m6A methylation experiments.

### In Vitro m6A Methylation and Interaction Assays

All plasmids and oligonucleotides used in this assay are listed in Table 8. Using PCR, a DNA fragment was generated for Domain 2 of wild-type (pTRE3G-HOTAIR, pAJ171) and A783U (pTRE3G-A783U_HOTAIR, pAJ385) mutant HOTAIR using primers MB88 and MB89. A 5′ T7 promoter and 3′ RAT tag were added to the sequence via PCR with primers MB22 and MB94. In vitro transcription of the PCR templates was completed using the MEGAScript T7 Transcription Kit (AM1334, ThermoFisher Scientific) according to the manufacturer's instructions, RNA was purified using the RNeasy Mini Kit (Qiagen 75106) and quantified by UV. 500 nM RNA was diluted in 1× Methyltransferase Buffer (20 mM Tris pH 7.5, 0.01% Triton-X 100, 1 mM DTT) in reactions containing 50 μM SAM and 500 nM purified METTL3/14 (+m6A) for 1 hour at room temperature. Control reactions contained no METTL3/14 (−m6A). RNA was purified using the RNeasy Mini Kit according to manufacturer's instructions and quantified by UV.

To obtain FLAG-tagged YTHDC1 protein, 293 cells were transfected using Lipofectamine 2000 (11668030, ThermoFisher Scientific) with plasmid YTHDC1-FLAG and cell lysates were generated as previously described. Dynabeads (M270, Invitrogen) were resuspended in high-quality dry Dimethylformamide at a concentration of 2×10⁹ beads/ml. Dynabeads were stored at 4° C. and equilibrated to room temperature prior to use. Dynabeads were washed in 0.1 M Sodium Phosphate Buffer (pH 7.4) and vortexed for 30 seconds. A second wash was repeated with vortexing and incubation at room temperature for 10 minutes with rotation. 1 mg/ml IgG solution was prepared by diluting rabbit IgG (15006, Sigma) in 0.1 M Sodium Phosphate Buffer. Washed beads were resuspended in 0.1 M Sodium Phosphate Buffer at a concentration of 3×10⁹ beads/ml, and an equal volume of 1 mg/ml IgG was added. Samples were vortexed briefly and an equal volume of 3M Ammonium Sulfate was added and samples were mixed well. Samples were incubated at 37° C. for 18-24 hours with rotation. Samples were washed once briefly with 0.1 M Sodium Phosphate Buffer, then twice with incubation at room temperature for 10 minutes with rotation. Samples were washed in Sodium Phosphate Buffer+1% TritonX-100 at 37° C. for 10 minutes with rotation. A quick wash with 0.1 M Sodium Phosphate Buffer was performed and followed by 4 washes in 0.1 M Citric Acid pH 3.1 at a concentration of 2×10⁸ beads/ml at room temperature for 10 minutes with rotation. After a quick wash with 0.1 M Sodium Phosphate Buffer, beads were resuspended to 1×10⁹ beads/ml in 1×PBS+0.02% Sodium Azide and stored at 4° C. prior to use.

800 ng of +/−m6A RNA was incubated with 150 ng PrA-PP7 fusion protein in HLB300 (20 mM Hepes pH 7.9, 300 mM sodium chloride, 2 mM magnesium chloride, 0.1% NP-40, 10% glycerol, 0.1 mM PMSF, 0.5 mM DTT). RNA was prebound to PP7 for 30 minutes at 25° C., 1350 rpm. 75 μl IgG-coupled Dynabeads were washed with HLB300 twice and resuspended in 250 μl HLB300. 50 μl beads were added to each tube of RNA-PP7 and samples were incubated 1 hour at 25° C., 1350 rpm. Beads were washed twice with HLB300 and resuspended in 80 μl Binding Buffer (10 mM Hepes pH 7.4, 150 mM potassium chloride, 3 mM magnesium chloride, 2 mM DTT, 0.5% NP-40, 10% glycerol, 1 mM PMSF, 1× protease inhibitors) containing 80 U RNase Inhibitor. 25 μg YTHDC1-FLAG containing lysate and 800 ng competitor RNA (IVT untagged HOTAIR D2) was added to each sample. Samples were incubated at 4° C. for 2.5 hours on a rotor. Beads were washed 3 times with cold Wash Buffer (200 mM Tris-HCl pH 7.4, 200 mM sodium chloride, 2 mM magnesium Chloride, 1 mM DTT, 1× protease inhibitors) and resuspended in 1×SDS loading buffer. A 10% protein input sample was diluted in 1×SDS loading buffer. Samples were boiled 5 minutes at 95° C. and supernatant transferred to a new tube. Half of each sample was loaded on a 10% acrylamide gel and Western Blot was performed using anti-FLAG antibody.

TABLE 8

| Fragment | Template | F Primer | R Primer |
|---|---|---|---|
| WT HOTAIR | pAJ249 | MB88 | MB89 |
| D2 | pAJ385 | GTAATACGACTCAC | CCATATAAACTCCT |
| A783U | | TATAGGGAGCCAG | TAAAGCTTATATTT |
| HOTAIR | | AGGAG | TACAGTCC |
| D2 | | SEQ ID NO: 72 | SEQ ID NO: 73 |
| RAT-WT | WT | MB22 | MB94 |
| D2 | HOTAIR | TAATACGACTCACT | CGATGGCACGAGT |
| RAT-A783U | D2 | ATAGGG | GTAGCTAAACCTCG |
| D2 | A783U | SEQ ID NO: 74 | TGCCGACGTCTAAG |
| | HOTAIR | | GGTTTCCATATAAA |
| | D2 | | CTCCTTAAAGCTT |
| | | | SEQ ID NO: 75 |

Fractionation. Cells were grown in 15-cm dishes to 70-90% confluency. Cells were released with Trypsin (Corning), washed once with 1×PBS containing 1 mM EDTA, and split into two volumes. 1/4 of the sample was harvested in TRIzol and RNA isolated with RNeasy kit for the input RNA sample. The remaining 3/4 of the sample was fractionated into cytoplasmic, nucleoplasmic, and chromatin-associated samples. Cells were lysed in cold Cell Lysis Buffer (10 mM Tris-HCl pH 7.5, 0.15% NP-40, 150 mM Sodium Chloride) containing RNase inhibitors for 5 minutes on ice. Lysate was layered onto 2.5 volumes of Sucrose Cushion (10 mM Tris-HCl pH7.5, 150 mM Sodium Chloride, 24% Sucrose) containing RNase inhibitors. Samples were centrifuged for 10 minutes at 17,000×g at 4° C. Supernatant was collected (Cytoplasmic sample). Pellet was rinsed with 1×PBS containing 1 mM EDTA and resuspended in cold Glycerol Buffer (20 mM Tris-HCl pH 7.9, 75 mM Sodium Chloride, 0.5 mM EDTA, 0.85 mM DTT, 0.125 mM PMSF, 50% Glycerol) containing RNase inhibitors. An equal volume of cold Nuclei Lysis Buffer (10 mM HEPES pH 7.6, 1 mM DTT, 7.5 mM Magnesium Chloride, 0.2 mM EDTA, 0.3 M Sodium Chloride, 1M Urea, 1% NP-40) was added and sample was briefly vortexed twice for 2 seconds. Samples were incubated on ice 2 minutes and centrifuged for 2 minutes at 17,000×g at 4° C. Supernatant was collected (Nucleoplasmic sample). The remaining pellet was resuspended in 1×PBS containing 1 mM EDTA (Chromatin-associated sample). Each sample was subjected to TURBO DNase digestion at 37° C. for 30 minutes in 1× TURBO Buffer and 10 U TURBO for cytoplasmic and nucleoplasmic samples, or 40 U TURBO for chromatin-associated sample. Reactions were quenched with 10 mM EDTA and 3 volumes of TRIzol-LS was added. RNA isolation was performed as recommended by manufacturer. Samples were quantified by nanodrop to determine RNA concentration and ran on a 2% agarose gel to confirm RNA integrity. qRT-PCR was performed on 2 μg of RNA and normalized to RNA recovery, input values, and GAPDH.

Luciferase Assay. Analysis of luciferase activity was performed using the Luciferase Assay System (E1500, Promega). Cells were washed with 1×PBS and lysed in 100 μl 1× Cell Culture Lysis Reagent. Cells were scraped from bottom of dish and suspension was transferred to a new tube. Lysates were frozen and thawed prior to luciferase assay to ensure complete lysis. Luciferase assays were performed on 20 μl of lysate or 1× Cell Culture Lysis Reagent in 96 well plates on the GloMax-Multi Detection System (TM297, Promega). 100 μl Luciferase Assay Reagent was added to wells, mixed, and light production measured. Measurements were performed in 3 technical replicates for each biological replicate. Luciferase activity was normalized to protein concentration of samples.

siRNA Transfection. Silencer Select siRNAs were obtained from ThermoFisher targeting YTHDC1 (n372360, n372361, n372362) or Negative Controls (U.S. Pat. Nos. 4,390,843, 4,390,846) and transfected into 293 cell lines using Lipofectamine RNAiMAX Transfection Reagent (13778030, ThermoFisher). Transfections were performed in a 24-well plate with 5 pmol of siRNA and 1.5 μl RNAiMAX Transfection reagent per well. Cells were harvested 24 hours after transfections and analyzed by Luciferase Assay and qRT-PCR.

Gene Expression Analyses. Total RNA was extracted from MDA-MB-231 cells using TRIZol (Life Technologies) with extraction in chloroform followed by purification with the RNeasy kit (Qiagen). Samples were DNase treated using TURBO DNase (Ambion). polyA-selected sequencing libraries were prepared and sequenced. All gene expression data associated with this publication are available through GEO accession number GSE173530. Differential gene expression analysis was performed using Salmon and DESeq2. Briefly, the reads were quantified using salmon to generate transcript abundance estimates and then DESeq2 was used to determine differential expression between samples. Heat maps were generated by using normalized read counts of genes that were significantly (p<0.1) differentially expressed between conditions to generate Z-scores. GO term enrichment analysis was performed using the GO Consortium's online PANTHER tool. To analyze correlation between expression in HOTAIR v. Control and A783U v. Control pairwise comparisons, the total set of differentially expressed genes were filtered to include only those whose fold change value was greater than 1.15 in either direction for both comparisons. These values were then plotted against each other. Linear regression was used to fit a trend line over the points, with the calculated Pearson correlation coefficient included in the graph.

dCasRX-YTHDC1 and gRNA Transfection. One plasmid containing dCasRX-FLAG-YTHDC1 in pXR002 in combination with one plasmid containing the designated guide RNA in pXR003 (see description in Plasmid Construction) were transfected into a 70-90% confluent 10-cm dish using Lipofectamine 2000 (11668030, Invitrogen) according to manufacturer's instructions. Plates were incubated at 37° C. for ˜24 hours, then subjected to fractionation as described herein.

Statistical Analyses. Graphs were prepared and data fitting and statistical analyses were performed using Biovinci (version 1.1.5, Bioturing, Inc., San Diego, California, USA). Each box-and-whisker plot displays datapoints for each replicate, the median value as a line, a box around the lower and upper quartiles, and whiskers extending to maximum and minimum values, excluding outliers as determined by the upper and lower fences. A student's 2-tailed paired T-test was used to determine the statistical significance between two samples. Differences and relationships were considered statistically significant when p≤0.05. For all graphs, *p<0.05, **p<0.01, ***p<0.001, ****p<0.0001.

All the compositions and methods disclosed and claimed herein can be made and executed without undue experimentation in light of the present disclosure. While the compositions and methods have been described in terms of embodiments, it is apparent to those of skill in the art that variations can be applied to the compositions and methods and in the steps or in the sequence of steps of the methods described herein without departing from the concept, spirit and scope herein. More specifically, certain agents that are both chemically and physiologically related can be substituted for the agents described herein while the same or similar results would be achieved. All such similar substitutes and modifications apparent to those skilled in the art are deemed to be within the spirit, scope and concept as defined by the appended claims.

1-8. (canceled)

9. An antisense oligonucleotide (ASO) capable of pairing with HOTAIR (HOX (homeobox) transcript antisense intergenic RNA) comprising an ASO capable of binding to, or interfering with, methylation of HOTAIR position A783 represented by the polynucleotide of SEQ ID. NO: 1; or equivalent position thereof; and reducing or blocking methylation of position A783 represented by the polynucleotide of SEQ ID. NO: 1; or equivalent position thereof.

10. The ASO according to claim 9, wherein the ASO comprises ten (10) to about twenty (20) nucleotides.

11. The ASO according to claim 9, wherein the ASO comprises a uracil that binds to adenosine at position 783 represented by the polynucleotide of SEQ ID. NO: 1; or equivalent position thereof.

12. The ASO according to claim 9, wherein the ASO comprises a polynucleotide represented by at least one of SEQ. ID NO. 5-SEQ. ID NO. 24 or mixture thereof.

13. The ASO according to claim 9, wherein the ASO further comprises a cell targeting agent or moiety.

14. The ASO according to claim 9, wherein the ASO further comprises at least one chemical modification to the ASO of its phosphodiester backbone.

15. The ASO according to claim 14, wherein the at least one chemical modification to the ASO phosphodiester backbone comprises at least one of phosphorothioate DNA, phosphorodiamidate morpholino (PMO), peptide nucleic acid, tricyclo-DNA, ribose substitution 2′-O-methyl (2′-OMe), ribose substitution 2′-O-methoxyethyl (2′-MOE), ribose substitution locked nucleic acid, or any combination thereof.

16. A composition comprising the anti-sense oligonucleotide according to claim 9; and a buffer.

17. The composition according to claim 16, further comprising a pharmaceutically acceptable excipient.

18. The composition according to claim 17, further comprising a buffer for stabilizing the anti-sense oligonucleotide.

19. A method for treating cancer in a subject, the method comprising administering a composition according to claim 17 to the subject and treating the cancer in the subject, wherein the cancer comprises a cancer having solid tumors or tumors expressing or over-expressing HOTAIR.

20. The method according to claim 19, wherein the cancer comprises a cancer having solid tumors over-expressing HOTAIR.

21. The method according to claim 19, wherein the cancer comprises one or more of breast, endometrial, prostate, pancreatic, glioma, lung, liver, cervical, colon, stomach, intestinal, other gastric cancer, or any combination thereof.

22. The method according to claim 19, wherein the cancer comprises breast cancer and the treatment reduces expansion and/or metastasis of the breast cancer cells in the subject.

23. (canceled)

24. The method according to claim 22, wherein the composition reduces one or more of HOTAIR cancer-promoting activity or activities, reduces HOTAIR expression, and induces cancer suppression in the subject to treat the cancer in the subject.

25. The method according to claim 19, further comprising treating the subject at least one of before, during or after treating the subject with the composition with one or more of radiation, surgery, chemotherapy, immunotherapy, hormone therapy, stem cell therapy, bone marrow transplantation, or other method for treating cancer.

26. (canceled)

27. The method according to any one of claims 19-26, wherein administering the composition comprises administering the composition intravenously, by continuous infusion over a predetermined period, by slow-release formulation or time-release formulation, subcutaneously, intraocularly, topically, intranasally or other mode.

28. The method according to claim 19, wherein administering the composition comprises administering daily, every other day, bi-weekly, weekly, bi-monthly, monthly, every other month or other time period determined by a healthcare provider.

29. The method according to claim 19, further comprising reducing expression of or inhibiting activity of YTHDC1.

30-36. (canceled)

36. A kit comprising the anti-sense oligonucleotide according to claim 9; and at least one container.

37. The ASO according to claim 9, wherein the ASO comprises at least ten (10) consecutive nucleotides of SEQ ID NO: 76.


SEQUENCE LISTING (24 sequences)

SEQ ID NO: 1 - RNA, 2148 nt, Homo sapiens
  source 1..2148: mol_type = other RNA; note = wildtype_HOTAIR_transcript
gactcgcctgtgctctggagcttgatccgaaagcttccacagtgaggactgctccgtgggggtaagagagcaccaggcactgaggcctgggagttccacagaccaacacccctgctcctggcggctcccacccggggcttagaccctcaggtccctaatatcccggaggtgctctcaatcagaaaggtcctgctccgcttcgcagtggaatggaacggatttagaagcctgcagtaggggagtggggagtggagagagggagcccagagttacagacggcggcgagaggaaggaggggcgtctttatttttttaaggccccaaagagtctgatgtttacaagaccagaaatgccacggccgcgtcctggcagagaaaaggctgaaatggaggaccggcgccttccttataagtatgcacattggcgagagaattaagtgctgcaacctaaaccagcaattacacccaagctcgttggggcctaagccagtaccgacctggtagaaaaagcaaccacgaagctagagagagagccagaggagggaagagagcgccagacgaaggtgaaagcgaaccacgcagagaaatgcaggcaagggagcaaggcggcagttcccggaacaaacgtggcagagggcaagacgggcactcacagacagaggtttatgtatttttattttttaaaatctgatttggtgttccatgaggaaaagggaaaatctagggaacgggagtacagagagaataatccgggtcctagctcgccacatgaacgcccagagaacgctggaaaaacctgagcgggtgccggggcagcacccggctcgggtcagccactgccccacaccgggcccaccaagccccgcccctcgcggccaccggggcttccttgctcttcttatcatctccatctttatgatgaggcttgttaacaagaccagagagctggccaagcacctctatctcagccgcgcccgctcagccgagcagcggtcggtggggggactgggaggcgctaattaattgattcctttggactgtaaaatatggcggcgtctacacggaacccatggactcataaacaatatatctgttgggcgtgagtgcactgtctctcaaataatttttccataggcaaatgtcagagggttctggatttttagttgctaaggaaagatccaaatgggaccaattttaggaggcccaaacagagtccgttcagtgtcagaaaatgcttccccaaagggttggcagtgtgttttgttggaaaaaagcttgggttataggaaagcctttccctgctacttgtgtagacccagcccaatttaagaattacaaggaagcgaaggggttgtgtaggccggaagcctctctgtcccggctggatgcaggggacttgagctgctccggaatttgagaggaacatagaagcaaaggtccagcctttgcttcgtgctgattcctagacttaagattcaaaaacaaatttttaaaagtgaaaccagccctagcctttggaagctcttgaaggttcagcacccacccaggaatccacctgcctgttacacgcctctccaagacacagtggcaccgcttttctaactggcagcacagagcaactctataatatgcttatattaggtctagaagaatgcatcttgagacacatgggtaacctaattatataatgcttgttccatacaggagtgattatgcagtgggaccctgctgcaaacgggactttgcactctaaatataggccccagcttgggacaaaagttgcagtagaaaaatagacataggagaacacttaaataagtgatgcatgtagacacagaaggggtatttaaaagacagaaataatagaagtacagaagaacagaaaaaaaatcagcagatggagattaccattcccaatgcctgaacttcctcctgctattaagattgctagagaattgtgtcttaaacagttcatgaacccagaagaacgcaatttcaatgtatttagtacacacacagtatgtatataaacacaactcacagaatatattttccatacattgggtaggtatgcactttgtgtatatataataatgtattttccatgcagttttaaaatgtagatatattaatatctggatgcattttc

SEQ ID NO: 2 - RNA, 2148 nt, synthetic construct
  source 1..2148: mol_type = other RNA; note = mutant_HOTAIR_transcript
gactcgcctgtgctctggagcttgatccgaaagcttccacagtgaggactgctccgtgggggtaagagagcaccaggcactgaggcctgggagttccacagaccaacacccctgctcctggcggctcccacccggggcttagaccctcaggtccctaatatcccggaggtgctctcaatcagaaaggtcctgctccgcttcgcagtggaatggaacggatttagaagcctgcagtaggggagtggggagtggagagagggagcccagagttacagacggcggcgagaggaaggaggggcgtctttatttttttaaggccccaaagagtctgatgtttacaagaccagaaatgccacggccgcgtcctggcagagaaaaggctgaaatggaggaccggcgccttccttataagtatgcacattggcgagagaattaagtgctgcaacctaaaccagcaattacacccaagctcgttggggcctaagccagtaccgacctggtagaaaaagcaaccacgaagctagagagagagccagaggagggaagagagcgccagacgaaggtgaaagcgaaccacgcagagaaatgcaggcaagggagcaaggcggcagttcccggaacaaacgtggcagagggcaagacgggcactcacagacagaggtttatgtatttttattttttaaaatctgatttggtgttccatgaggaaaagggaaaatctagggaacgggagtacagagagaataatccgggtcctagctcgccacatgaacgcccagagatcgctggaaaaacctgagcgggtgccggggcagcacccggctcgggtcagccactgccccacaccgggcccaccaagccccgcccctcgcggccaccggggcttccttgctcttcttatcatctccatctttatgatgaggcttgttaacaagaccagagagctggccaagcacctctatctcagccgcgcccgctcagccgagcagcggtcggtggggggactgggaggcgctaattaattgattcctttggactgtaaaatatggcggcgtctacacggaacccatggactcataaacaatatatctgttgggcgtgagtgcactgtctctcaaataatttttccataggcaaatgtcagagggttctggatttttagttgctaaggaaagatccaaatgggaccaattttaggaggcccaaacagagtccgttcagtgtcagaaaatgcttccccaaagggttggcagtgtgttttgttggaaaaaagcttgggttataggaaagcctttccctgctacttgtgtagacccagcccaatttaagaattacaaggaagcgaaggggttgtgtaggccggaagcctctctgtcccggctggatgcaggggacttgagctgctccggaatttgagaggaacatagaagcaaaggtccagcctttgcttcgtgctgattcctagacttaagattcaaaaacaaatttttaaaagtgaaaccagccctagcctttggaagctcttgaaggttcagcacccacccaggaatccacctgcctgttacacgcctctccaagacacagtggcaccgcttttctaactggcagcacagagcaactctataatatgcttatattaggtctagaagaatgcatcttgagacacatgggtaacctaattatataatgcttgttccatacaggagtgattatgcagtgggaccctgctgcaaacgggactttgcactctaaatataggccccagcttgggacaaaagttgcagtagaaaaatagacataggagaacacttaaataagtgatgcatgtagacacagaaggggtatttaaaagacagaaataatagaagtacagaagaacagaaaaaaaatcagcagatggagattaccattcccaatgcctgaacttcctcctgctattaagattgctagagaattgtgtcttaaacagttcatgaacccagaagaacgcaatttcaatgtatttagtacacacacagtatgtatataaacacaactcacagaatatattttccatacattgggtaggtatgcactttgtgtatatataataatgtattttccatgcagttttaaaatgtagatatattaatatctggatgcattttc

SEQ ID NO: 3 - DNA, 2364 bp, Homo sapiens
  misc_feature 1..2364: note = HOX transcript antisense RNA (HOTAIR), transcript variant 2, long non-coding RNA
  source 1..2364: mol_type = other DNA
cctccaggccctgccttctgcctgcacattctgccctgatttccggaacctggaagcctaggcaggcagtggggaactctgactcgcctgtgctctggagcttgatccgaaagcttccacagtgaggactgctccgtgggggtaagagagcaccaggcactgaggcctgggagttccacagaccaacacccctgctcctggcggctcccacccgggacttagaccctcaggtccctaatatcccggaggtgctctcaatcagaaaggtcctgctccgcttcgcagtggaatggaacggatttagaagcctgcagtaggggagtggggagtggagagagggagcccagagttacagacggcggcgagaggaaggaggggcgtctttatttttttaaggccccaaagagtctgatgtttacaagaccagaaatgccacggccgcgtcctggcagagaaaaggctgaaatggaggaccggcgccttccttataagtatgcacattggcgagagaagtgctgcaacctaaaccagcaattacacccaagctcgttggggcctaagccagtaccgacctggtagaaaaagcaaccacgaagctagagagagagccagaggagggaagagagcgccagacgaaggtgaaagcgaaccacgcagagaaatgcaggcaagggagcaaggcggcagttcccggaacaaacgtggcagagggcaagacgggcactcacagacagaggtttatgtatttttattttttaaaatctgatttggtgttccatgaggaaaagggaaaatctagggaacgggagtacagagagaataatccgggtcctagctcgccacatgaacgcccagagaacgctggaaaaacctgagcgggtgccggggcagcacccggctcgggtcagccactgccccacaccgggcccaccaagccccgcccctcgcggccaccggggcttccttgctcttcttatcatctccatctttatgatgaggcttgttaacaagaccagagagctggccaagcacctctatctcagccgcgcccgctcagccgagcagcggtcggtggggggactgggaggcgctaattaattgattcctttggactgtaaaatatggcggcgtctacacggaacccatggactcataaacaatatatctgttgggcgtgagtgcactgtctctcaaataatttttccataggcaaatgtcagagggttctggatttttagttgctaaggaaagatccaaatgggaccaattttaggaggcccaaacagagtccgttcagtgtcagaaaatgcttccccaaaggggttgggagtgtgttttgttggaaaaaagcttgggttataggaaagcctttccctgctacttgtgtagacccagcccaatttaagaattacaaggaagcgaaggggttgtgtaggccggaagcctctctgtcccggctggatgcaggggacttgagctgctccggaatttgagaggaacatagaagcaaaggtccagcctttgcttcgtgctgattcctagacttaagattcaaaaacaaatttttaaaagtgaaaccagccctagcctttggaagctcttgaaggttcagcacccacccaggaatccacctgcctgttacacgcctctccaagacacagtggcaccgcttttctaactggcagcacagagcaactctataatatgcttatattaggtctagaagaatgcatcttgagacacatgggtaacctaattatataatgcttgttccatacaggagtgattatgcagtgggaccctgctgcaaacgggactttgcactctaaatatagaccccagcttgggacaaaagttgcagtagaaaaatagacataggagaacacttaaataagtgatgcatgtagacacagaaggggtatttaaaagacagaaataatagaagtacagaagaacagaaaaaaaatcagcagatggagattaccattcccaatgcctgaacttcctcctgctattaagattgctagagaattgtgtcttaaacagttcatgaacccagaagaatgcaatttcaatgtatttagtacacacacagtatgtatataaacacaactcacagaatatattttccatacattgggtaggtatgcactttgtgtatatataataatgtattttccatgcagttttaaaatgtagatatattaatatctggatgcattttctgtgcactggttttatatgccttatggagtatatactcacatgtagctaaatagactcaggactgcacattccttgtgtaggttgtgtgtgtgtggtggttttatgcataaataaagttttacatgtggtgaatataaa

SEQ ID NO: 4 - RNA, 2148 nt, synthetic construct
  source 1..2148: mol_type = other RNA
  misc_feature 48: note = n = adenine (A) or uracil (U)
  misc_feature 102: note = n = adenine (A) or uracil (U)
  misc_feature 143: note = n = adenine (A) or uracil (U)
  misc_feature 215: note = n = adenine (A) or uracil (U)
  misc_feature 557: note = n = adenine (A) or uracil (U)
  misc_feature 620: note = n = adenine (A) or uracil (U)
  misc_feature 655: note = n = adenine (A) or uracil (U)
  misc_feature 772: note = n = adenine (A) or uracil (U)
  misc_feature 783: note = n = adenine (A) or uracil (U)
  misc_feature 936: note = n = adenine (A) or uracil (U)
  misc_feature 1394: note = n = adenine (A) or uracil (U)
  misc_feature 1579: note = n = adenine (A) or uracil (U)
  misc_feature 1663: note = n = adenine (A) or uracil (U)
  misc_feature 1722: note = n = adenine (A) or uracil (U)
  misc_feature 1739: note = n = adenine (A) or uracil (U)
gactcgcctgtgctctggagcttgatccgaaagcttccacagtgaggnctgctccgtgggggtaagagagcaccaggcactgaggcctgggagttccacagnccaacacccctgctcctggcggctcccacccggggcttagnccctcaggtccctaatatcccggaggtgctctcaatcagaaaggtcctgctccgcttcgcagtggaatggancggatttagaagcctgcagtaggggagtggggagtggagagagggagcccagagttacagacggcggcgagaggaaggaggggcgtctttatttttttaaggccccaaagagtctgatgtttacaagaccagaaatgccacggccgcgtcctggcagagaaaaggctgaaatggaggaccggcgccttccttataagtatgcacattggcgagagaattaagtgctgcaacctaaaccagcaattacacccaagctcgttggggcctaagccagtaccgacctggtagaaaaagcaaccacgaagctagagagagagccagaggagggaagagagcgccagncgaaggtgaaagcgaaccacgcagagaaatgcaggcaagggagcaaggcggcagttcccggancaaacgtggcagagggcaagacgggcactcacagncagaggtttatgtatttttattttttaaaatctgatttggtgttccatgaggaaaagggaaaatctagggaacgggagtacagagagaataatccgggtcctagctcgccacatgancgcccagagancgctggaaaaacctgagcgggtgccggggcagcacccggctcgggtcagccactgccccacaccgggcccaccaagccccgcccctcgcggccaccggggcttccttgctcttcttatcatctccatctttatgatgaggcttgttaacaagnccagagagctggccaagcacctctatctcagccgcgcccgctcagccgagcagcggtcggtggggggactgggaggcgctaattaattgattcctttggactgtaaaatatggcggcgtctacacggaacccatggactcataaacaatatatctgttgggcgtgagtgcactgtctctcaaataatttttccataggcaaatgtcagagggttctggatttttagttgctaaggaaagatccaaatgggaccaattttaggaggcccaaacagagtccgttcagtgtcagaaaatgcttccccaaagggttggcagtgtgttttgttggaaaaaagcttgggttataggaaagcctttccctgctacttgtgtagacccagcccaatttaagaattacaaggaagcgaaggggttgtgtaggccggaagcctctctgtcccggctggatgcaggggncttgagctgctccggaatttgagaggaacatagaagcaaaggtccagcctttgcttcgtgctgattcctagacttaagattcaaaaacaaatttttaaaagtgaaaccagccctagcctttggaagctcttgaaggttcagcacccacccaggaatccacctgcctgttacacgcctctccaagncacagtggcaccgcttttctaactggcagcacagagcaactctataatatgcttatattaggtctagaagaatgcatcttgagncacatgggtaacctaattatataatgcttgttccatacaggagtgattatgcagtgggnccctgctgcaaacgggnctttgcactctaaatataggccccagcttgggacaaaagttgcagtagaaaaatagacataggagaacacttaaataagtgatgcatgtagacacagaaggggtatttaaaagacagaaataatagaagtacagaagaacagaaaaaaaatcagcagatggagattaccattcccaatgcctgaacttcctcctgctattaagattgctagagaattgtgtcttaaacagttcatgaacccagaagaacgcaatttcaatgtatttagtacacacacagtatgtatataaacacaactcacagaatatattttccatacattgggtaggtatgcactttgtgtatatataataatgtattttccatgcagttttaaaatgtagatatattaatatctggatgcattttc

SEQ ID NO: 5 - DNA, 20 bp, synthetic construct
  misc_feature 1..20: note = synthetic construct - antisense oligonucleotide
  source 1..20: mol_type = other DNA
cgctcaggtttttccagcgt

SEQ ID NO: 6 - DNA, 20 bp, synthetic construct
  misc_feature 1..20: note = synthetic construct - antisense oligonucleotide
  source 1..20: mol_type = other DNA
gctcaggtttttccagcgtt

SEQ ID NO: 7 - DNA, 20 bp, synthetic construct
  misc_feature 1..20: note = synthetic construct - antisense oligonucleotide
  source 1..20: mol_type = other DNA
ctcaggtttttccagcgttc

SEQ ID NO: 8 - DNA, 20 bp, synthetic construct
  misc_feature 1..20: note = synthetic construct - antisense oligonucleotide
  source 1..20: mol_type = other DNA
tcaggtttttccagcgttct

SEQ ID NO: 9 - DNA, 20 bp, synthetic construct
  misc_feature 1..20: note = synthetic construct - antisense oligonucleotide
  source 1..20: mol_type = other DNA
caggtttttccagcgttctg

SEQ ID NO: 10 - DNA, 20 bp, synthetic construct
  misc_feature 1..20: note = synthetic construct - antisense oligonucleotide
  source 1..20: mol_type = other DNA
aggtttttccagcgttctct

SEQ ID NO: 11 - DNA, 20 bp, synthetic construct
  misc_feature 1..20: note = synthetic construct - antisense oligonucleotide
  source 1..20: mol_type = other DNA
ggtttttccagcgttctctg

SEQ ID NO: 12 - DNA, 20 bp, synthetic construct
  misc_feature 1..20: note = synthetic construct - antisense oligonucleotide
  source 1..20: mol_type = other DNA
gtttttccagcgttctctgg

SEQ ID NO: 13 - DNA, 20 bp, synthetic construct
  misc_feature 1..20: note = synthetic construct - antisense oligonucleotide
  source 1..20: mol_type = other DNA
tttttccagcgttctctggg

SEQ ID NO: 14 - DNA, 20 bp, synthetic construct
  misc_feature 1..20: note = synthetic construct - antisense oligonucleotide
  source 1..20: mol_type = other DNA
ttttccagcgttctctgggc

SEQ ID NO: 15 - DNA, 20 bp, synthetic construct
  misc_feature 1..20: note = synthetic construct - antisense oligonucleotide
  source 1..20: mol_type = other DNA
tttccagcgttctctgggcg

SEQ ID NO: 16 - DNA, 20 bp, synthetic construct
  misc_feature 1..20: note = synthetic construct - antisense oligonucleotide
  source 1..20: mol_type = other DNA
ttccagcgttctctgggcgt

SEQ ID NO: 17 - DNA, 20 bp, synthetic construct
  misc_feature 1..20: note = synthetic construct - antisense oligonucleotide
  source 1..20: mol_type = other DNA
tccagcgttctctgggcgtt

SEQ ID NO: 18 - DNA, 20 bp, synthetic construct
  misc_feature 1..20: note = synthetic construct - antisense oligonucleotide
  source 1..20: mol_type = other DNA
ccagcgttctctgggcgttc

SEQ ID NO: 19 - DNA, 20 bp, synthetic construct
  misc_feature 1..20: note = synthetic construct - antisense oligonucleotide
  source 1..20: mol_type = other DNA
cagcgttctctgggcgttca

SEQ ID NO: 20 - DNA, 20 bp, synthetic construct
  misc_feature 1..20: note = synthetic construct - antisense oligonucleotide
  source 1..20: mol_type = other DNA
agcgttctctgggcgttcat

SEQ ID NO: 21 - DNA, 20 bp, synthetic construct
  misc_feature 1..20: note = synthetic construct - antisense oligonucleotide
  source 1..20: mol_type = other DNA
gcgttctctgggcgttcatg

SEQ ID NO: 22 - DNA, 20 bp, synthetic construct
  misc_feature 1..20: note = synthetic construct - antisense oligonucleotide
  source 1..20: mol_type = other DNA
cgttctctgggcgttcatgt

SEQ ID NO: 23 - DNA, 20 bp, synthetic construct
  misc_feature 1..20: note = synthetic construct - antisense oligonucleotide
  source 1..20: mol_type = other DNA
gttctctgggcgttcatgtg

SEQ ID NO: 24 - DNA, 20 bp, synthetic construct
  misc_feature 1..20: note = synthetic construct - antisense oligonucleotide
  source 1..20: mol_type = other DNA
ttctctgggcgttcatgtgg